(19) United States
(12) Patent Application Publication
(10) Pub. No.: US 2024/0182436 A1
(43) Pub. Date: Jun. 6, 2024

(54) SUBSTITUTED SULFONAMIDE-CHROMAN COMPOUNDS, AND PHARMACEUTICAL COMPOSITIONS, AND METHODS OF USE THEREOF

(71) Applicant: Sumitomo Pharma America, Inc., Marlborough, MA (US)

(72) Inventors: Nina DEDIC, Somerville, MA (US); Linghong XIE, Southborough, MA (US)

(73) Assignee: Sumitomo Pharma America, Inc., Marlborough, MA (US)

(21) Appl. No.: 18/554,211

(22) PCT Filed: Apr. 7, 2022
(86) PCT No.: PCT/US2022/071586

§ 371 Date: Oct. 6, 2023

Related U.S. Application Data
(60) Provisional application No. 63/173,369, filed on Apr. 10, 2021.

(51) Int. Cl. C07D 311/68; A61P 25/16; C07D 311/66

(52) U.S. Cl. CPC C07D 311/68; A61P 25/16; C07D 311/66

(57) ABSTRACT

Provided herein are compounds, pharmaceutical compositions, and methods of use thereof, including methods of treating neurological disorders. For example, provided herein is a compound of Formula I:

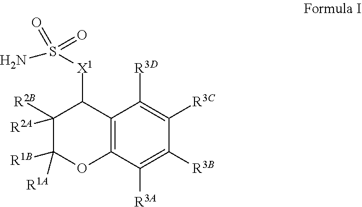

or a pharmaceutically acceptable salt thereof, wherein values for the variables (e.g., X¹, R^{1A}, R^{1B}, R^{2A}, R^{2B}, R^{3A}, R^{3B}, R^{3C}, R^{3D}) are as disclosed herein. The compounds disclosed herein (e.g., compounds of Formula I, or pharmaceutically acceptable salts thereof) and pharmaceutical compositions can be used to treat neurological disorders.

### CROSS REFERENCE TO RELATED APPLICATIONS

This application claims priority to U.S. Provisional Application No. 63/173,369, filed Apr. 10, 2021, the entire disclosure of which is hereby incorporated herein by reference.

### FIELD

The present disclosure relates to compounds, pharmaceutical compositions, and methods of use thereof, including methods of treating neurological or psychiatric diseases or disorders.

### BACKGROUND

Neurological and psychiatric diseases or disorders relate to a complex and confounding classification of conditions affecting a substantial proportion of the population with varying degrees of severity. For example, a class of neurological and psychiatric diseases or disorders is movement disorders, which include essential tremor. Essential tremor is considered to be a tremor syndrome characterized by isolated bilateral upper-limb action tremor with a duration of at least 3 years, with or without tremor in other locations, such as head, larynx (voice tremor), or lower limbs. Essential tremor may manifest with additional mild neurologic signs of diagnostic uncertainty, such as mild ataxia, questionably abnormal posturing of the limbs, or impaired memory, affecting 1% of the population worldwide (Haubenberger, D., et al. New Eng J Med, 378(19): 1802-1810). Additional neurological or psychiatric diseases or disorders are also described in the Diagnostic and Statistical Manual of Mental Disorders, Fifth Edition (2013) (DSM-V), and as regularly updated by the American Psychiatric Association.

While treatment for some neurological or psychiatric diseases or disorders exist, there remains a need for effective treatments for various neurological and psychiatric diseases and disorders. For example, propranolol, classified as a beta-blocker, is the only pharmaceutical treatment approved by the United States Food and Drug Administration (FDA) for treating essential tremor. Additionally, other pharmaceutical classes, such as the anticonvulsants or benzodiazepines, have been used for treating essential tremor. However, safer or more effective treatment options are needed.

### SUMMARY

In one aspect, the present disclosure provides compounds of Formula I

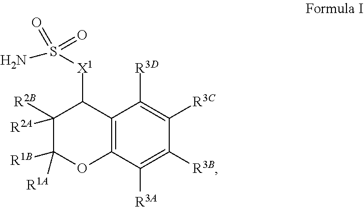

or a pharmaceutically acceptable salt thereof,
wherein:
   X¹ is C(R^{4A})(R^{4B}) or N(R^{4C})
      R^{4A} and R^{4B} are each independently H, (C₁-C₄)alkyl, (C₂-C₄)alkenyl, (C₂-C₄)alkynyl, (C₁-C₄)alkoxy, or (C₃-C₆)cycloalkyl; or
      R^{4A} and R^{4B}, together with the carbon atom to which they are attached, form a (C₃-C₆)cycloalkyl;
      R^{4C} is H, (C₁-C₄)alkyl, (C₂-C₄)alkenyl, (C₂-C₄)alkynyl, or (C₃-C₆)cycloalkyl;
   R^{1A}, R^{1B}, R^{2A}, and R^{2B} are each independently H, halogen, (C₁-C₄)alkyl, (C₂-C₄)alkenyl, (C₂-C₄)alkynyl, (C₁-C₄)alkoxy, (C₁-C₄)haloalkyl, or (C₃-C₆)cycloalkyl;
      or R^{1A} and R^{1B}, together with the carbon atom to which they are attached, form a spiro-(C₃-C₆)cycloalkyl;
      or R^{2A} and R^{2B}, together with the carbon atom to which they are attached, form a spiro-(C₃-C₆)cycloalkyl; and
   R^{3A}, R^{3B}, R^{3C}, and R^{3D} are each independently H, halogen, hydroxyl, (C₁-C₄)alkyl, (C₂-C₄)alkenyl, (C₂-C₄)alkynyl, (C₁-C₄)alkoxy, (C₁-C₄)haloalkyl, acyl, —CN, or (C₃-C₆)cycloalkyl;
   with the provisos that:
   when R^{1A}, R^{1B}, R^{2A}, R^{2B}, R^{3A}, R^{3B}, R^{3C}, and R^{3D} are each hydrogen, then (i) at least one of R^{4A} and R^{4B} is a group other than hydrogen or (ii) R^{4C} is a group other than hydrogen; and
   when X¹ is N(R^{4C}) and only one of R^{1A}, R^{1B}, R^{2A}, R^{2B}, R^{3A}, R^{3B}, R^{3C}, R^{3D}, and R^{4C} is a group other than hydrogen, then (i) R^{1A} is not methyl; (ii) R^{1B} is not methyl; and (iii) R^{3C} is not methoxy or ethoxy.

In another aspect, the invention relates to a pharmaceutical composition comprising a compound disclosed herein, or a pharmaceutically acceptable salt thereof, and a pharmaceutically acceptable excipient.

In another aspect, the invention relates to a method for treating a neurological or psychiatric disease or disorder in a subject, comprising administering to said subject an effective amount of a compound, or a pharmaceutically acceptable salt thereof, or pharmaceutical composition disclosed herein.

### DETAILED DESCRIPTION

A description of example embodiments follows.

Provided herein are definitions to assist with interpreting this disclosure. Whenever appropriate, terms used in the singular will also include the plural. Unless the context clearly indicates otherwise, terms used herein have the following meanings.

All methods described herein can be performed in any suitable order unless otherwise indicated herein or otherwise clearly contradicted by context. The use of any and all examples, or exemplary language (e.g., “such as”) provided herein is intended merely to better illuminate the present disclosure and does not pose a limitation on the scope of the present disclosure otherwise claimed.

As used herein, the term “acyl” refers to a group —C(═O)R wherein R is H, (C₁-C₄)alkyl, (C₁-C₄)alkenyl, (C₁-C₄)alkynyl or (C₃-C₆)cycloalkyl as defined herein. Examples of acyl include formyl, acetyl, and cyclohexylcarbonyl.

As used herein, the term “alkyl” refers to a branched or straight-chain, monovalent, hydrocarbon group having the specified number of carbon atoms, and the general formula CₙH₂ₙ₊₁. Thus, the term “(C₁-C₆)alkyl” refers to a branched or straight-chain, monovalent, hydrocarbon substituent of the general formula CₙH₂ₙ₊₁ wherein n is 1, 2, 3, 4, 5 or 6. Examples of alkyl include, but are not limited to, methyl, ethyl, n-propyl, i-propyl, n-butyl, i-butyl, s-butyl, t-butyl, n-pentyl, 1-methylbutyl, 2-methylbutyl, 3-methylbutyl, neopentyl, 3,3-dimethylpropyl, hexyl, 2-methylpentyl, and the like.

As used herein, the term “alkenyl” refers to an aliphatic group containing at least one carbon-carbon double bond and having from 2 to 4 carbon atoms (i.e., C₂-C₄ alkenyl). Examples of alkenyl groups include ethenyl, propenyl, butadienyl (including 1,2-butadienyl, and 1,3-butadienyl).

As used herein, the term “alkynyl” refers to aliphatic group containing at least one carbon-carbon triple bond and having from 2 to 4 carbon atoms (i.e., C₂-C₄ alkynyl). The term “alkynyl” also includes those groups having one triple bond and one double bond.

The term “alkoxy,” as used herein, refers to an alkyl group attached through an oxygen linking atom, wherein alkyl is as described herein. “(C₁-C₆)alkoxy” refers to an alkoxy group in which a (C₁-C₆)alkyl is attached through an oxygen linking atom. Examples of alkoxy groups include, but are not limited to, methoxy, ethoxy, propoxy (e.g., n-propoxy and iso-propoxy), and butoxy (e.g., t-butoxy).

“Halogen” and “halo,” as used herein, refer to fluorine, chlorine, bromine or iodine. In some embodiments, halogen is fluoro, chloro or bromo. In some embodiments, halogen is fluoro or chloro. In some embodiments, halogen is chloro, bromo or iodo. In some embodiments, halogen is chloro or bromo.

“Haloalkyl,” as used herein, refers to an alkyl group wherein one or more hydrogen atoms is each independently replaced by a halogen, wherein alkyl is as described herein. “Haloalkyl” includes mono-, poly- and perhaloalkyl groups. “(C₁-C₆)haloalkyl” refers to a (C₁-C₆)alkyl wherein one or more hydrogen atoms is each independently replaced by a halogen.

Examples of haloalkyl include, but are not limited to, fluoromethyl, difluoromethyl, trifluoromethyl, trichloromethyl, pentafluoroethyl, pentachloroethyl, 2,2,2 trifluoroethyl, heptafluoropropyl, and heptachloropropyl.

“Hydroxyl,” as used herein, means —OH.

“Cyano” or “—CN” as used herein, means —C≡N.

The term “cycloalkyl,” as used herein, refers to a saturated, monocyclic or polycyclic (e.g., bicyclic, tricyclic), aliphatic, hydrocarbon ring system having the specified number of carbon atoms. Thus, “(C₅-C₈)cycloalkyl” means a cycloalkyl ring system having from 5 to 8 ring carbons. Examples of cycloalkyl include, but are not limited to, cyclopropyl, cyclobutyl, cyclopentyl, cyclohexyl, and norbornyl.

The term “geminal,” (or abbreviated as “gem”), as used herein, refers to the relationship of substituent groups bonded to the same carbon. Examples of cycloalkyl include, but are not limited to, 1,1-dimethylcyclohexane, where cyclohexane is substituted with two geminal methyl substituents on the same carbon atom.

The term “spiro,” as used herein, refers to the relationship of two or more rings in which the adjoined rings share exactly one atom. Examples of cycloalkyl include, but are not limited to, spiro[2.5]octane, where cyclohexane is substituted with a spiro cyclopropyl substituent as an adjacent ring sharing the carbon atom.

The term “substituted,” as used herein, means that at least one (e.g., one, two, three, four, five, six, etc., from one to five, from one to three, one or two) hydrogen atom is replaced with a non-hydrogen substituent, provided that normal valencies are maintained and that the substitution results in a stable compound. Unless otherwise indicated, an “optionally substituted” group can have a substituent at each substitutable position of the group and, when more than one position in any given structure may be substituted with more than one substituent selected from a specified group, the substituent can be the same or different at every position. Alternatively, an “optionally substituted group” can be unsubstituted.

In cases wherein there are nitrogen atoms on compounds of the present disclosure, these nitrogen atoms may be converted to N-oxides by treatment with an oxidizing agent (e.g., mCPBA and/or hydrogen peroxide) to afford other compounds of this disclosure. Thus, shown and claimed nitrogen atoms are considered to cover both the shown nitrogen and its N-oxide (N→O) derivative.

When any variable occurs more than one time in any constituent or formula for a compound, its definition at each occurrence is independent of its definition at every other occurrence. Thus, for example, if a group is shown to be substituted with 0-3 substituents, then said group may be unsubstituted or substituted with up to three substituents, and each substituent is selected independently from the other substituent(s).

Combinations of substituents and/or variables are permissible only if such combinations result in stable compounds.

As a person of ordinary skill in the art would understand, for example, a ketone (—C(H)C(O)) group in a molecule may tautomerize to its enol form (—C═C(OH)). This disclosure is intended to cover all possible tautomers even when a structure depicts only one of them.

The phrase “pharmaceutically acceptable” means that the substance or composition the phrase modifies must be, within the scope of sound medical judgment, suitable for use in contact with the tissues of humans and lower animals without undue toxicity, irritation, allergic response and the like, and are commensurate with a reasonable benefit/risk ratio. If a substance is part of a composition or formulation, the substance must also be compatible chemically and/or toxicologically with the other ingredients in the composition or formulation.

Unless specified otherwise, the term “compounds of the present disclosure” refers to a compound of any structural formula depicted herein (e.g., a compound of Formula I, a subformula of a compound of Formula I, such as a compound of Formula I(A), I(B), II(A), II(B), III, III(A), III(B), IV, or IV(A)), as well as isomers, such as stereoisomers (including diastereoisomers, enantiomers and racemates), geometrical isomers, conformational isomers (including rotamers and atropisomers), tautomers, isotopically labeled compounds (including deuterium substitutions), and inherently formed moieties (e.g., polymorphs and/or solvates, such as hydrates) thereof. When a moiety is present that is capable of forming a salt, then salts are included as well, in particular, pharmaceutically acceptable salts. As used herein, and as would be understood by the person of skill in the art, the recitation of “a compound”—unless expressly further limited—is intended to include salts of that compound. In a particular embodiment, the term “compound” refers to the compound or a pharmaceutically acceptable salt thereof, this term refers to a pharmaceutically acceptable salt of the compound, even if not explicitly stated.

Compounds of the present disclosure may have asymmetric centers, chiral axes, and chiral planes (e.g., as described in: E. L. Eliel and S. H. Wilen, Stereo-chemistry of Carbon Compounds, John Wiley & Sons, New York, 1994, pages 1119-1190), and occur as racemic mixtures, individual isomers (e.g., diastereomers, enantiomers, geometrical isomers, conformational isomers (including rotamers and atropisomers), tautomers) and intermediate mixtures, with all possible isomers and mixtures thereof being included in the present disclosure.

As used herein, the terms “treatment,” “treat,” and “treating” refer to reversing, alleviating, delaying the onset of, inhibiting the progress of a disease or disorder, or managing one or more symptoms of a disease or disorder, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, the term “isomers” refers to different compounds that have the same molecular formula but differ in arrangement and configuration of the atoms. “Enantiomers” are a pair of stereoisomers that are non-superimposable mirror images of each other. A 1:1 mixture of a pair of enantiomers is a “racemic” mixture. “Racemate” or “racemic” is used to designate a racemic mixture where appropriate. When designating the stereochemistry for the compounds of the present disclosure, a single stereoisomer with known relative and absolute configuration of the two chiral centers can be designated using the conventional RS system (e.g., (1S,2S)); a single stereoisomer with known relative configuration but unknown absolute configuration is designated with stars (e.g., (R*), (S*), (1R*,2R*)); and a racemate with two letters (e.g., (1RS,2RS) as a racemic mixture of (1R,2R) and (1S,2S); (1RS,2SR) as a racemic mixture of (1R,2S) and (1S,2R)). “Diastereoisomers” are stereoisomers that have at least two asymmetric atoms, and which are not mirror-images of each other. The absolute stereochemistry can be specified according to the Cahn-Ingold-Prelog R-S system. When a compound is a pure enantiomer, the stereochemistry at each chiral carbon may be specified by either R or S. Resolved compounds can be designated (+) or (−) depending on the direction (dextro- or levorotatory) which they rotate plane polarized light at the wavelength of the sodium D line. Alternatively, the resolved compounds can be defined by the respective retention times for the corresponding enantiomers/diastereomers via chiral HPLC. Alternatively, graphic representations of racemic, ambiscalemic and scalemic or enantiomerically pure compounds used herein are a modified version of the denotations taken from Maehr J. Chem. Ed. 62, 114-120 (1985): simple lines provide no information about stereochemistry and convey only connectivity; solid and broken wedges are used to denote the absolute configuration of a chiral element; solid and broken bold lines indicated relative stereochemistry of indeterminate absolute configuration. For example, the graphic representation:

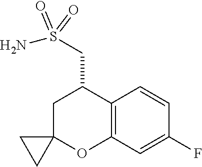 [SMILES: NS(=O)(=O)C[C@H]1CC2(CC2)Oc2cc(F)ccc21]

indicates one enantiomer of the pair of enantiomers represented as:

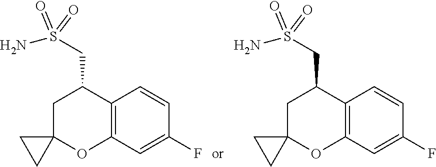 [SMILES: NS(=O)(=O)C[C@H]1CC2(CC2)Oc2cc(F)ccc21 ; NS(=O)(=O)C[C@@H]1CC2(CC2)Oc2cc(F)ccc21]

wherein the absolute configuration of the pair has not been established, and

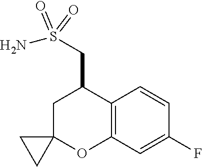 [SMILES: NS(=O)(=O)C[C@@H]1CC2(CC2)Oc2cc(F)ccc21]

is the enantiomer of

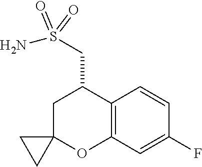 [SMILES: NS(=O)(=O)C[C@H]1CC2(CC2)Oc2cc(F)ccc21]

in any ratio, while the representation:

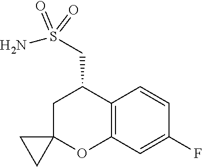 [SMILES: NS(=O)(=O)C[C@H]1CC2(CC2)Oc2cc(F)ccc21]

indicates a single enantiomer with the absolute configuration depicted, e.g., (S)-(7-fluorospiro[chromane-2,1′-cyclopropan]-4-yl)methanesulfonamide in the illustration above. Unless specified, each occurrence of a chiral center of compounds disclosed herein includes all possible stereoisomers.

The “enantiomeric excess” or “% enantiomeric excess” of a composition can be calculated using the equation shown below. In the example shown below, a composition contains 90% of one enantiomer, e.g., the S enantiomer, and 10% of the other enantiomer, e.g., the R enantiomer. In this example, % ee=(90−10)/100=80%.

Thus, a composition containing 90% of one enantiomer and 10% of the other enantiomer is said to have an enantiomeric excess of 80%. Some compositions described herein contain an enantiomeric excess of at least about 50%, 75%, 90%, 95%, or 99% of the S enantiomer. In other words, the compositions contain an enantiomeric excess of the S enantiomer over the R enantiomer. In other embodiments, some compositions described herein contain an enantiomeric excess of at least about 50%, 75%, 90%, 95%, or 99% of the R enantiomer. In other words, the compositions contain an enantiomeric excess of the R enantiomer over the S enantiomer.

For instance, an isomer/enantiomer can, in some embodiments, be provided substantially free of the corresponding enantiomer, and can also be referred to as “optically enriched,” “enantiomerically enriched,” “enantiomerically pure” and “non-racemic,” as used interchangeably herein. These terms refer to compositions in which the percent by weight of one enantiomer is greater than the amount of that one enantiomer in a control mixture of the racemic composition (e.g., greater than 1:1 by weight). For example, an enantiomerically enriched preparation of the S enantiomer, means a preparation of the compound having greater than about 50% by weight of the S enantiomer relative to the R enantiomer, such as at least about 75% by weight, further such as at least about 80% by weight. In some embodiments, the enrichment can be much greater than about 80% by weight, providing a “substantially enantiomerically enriched,” “substantially enantiomerically pure” or a “substantially non-racemic” preparation, which refers to preparations of compositions which have at least about 85% by weight of one enantiomer relative to other enantiomer, such as at least about 90% by weight, and further such as at least 95% by weight. In certain embodiments, the compound provided herein is made up of at least about 90% by weight of one enantiomer. In other embodiments, the compound is made up of at least about 95%, 98%, or 99% by weight of one enantiomer.

In some embodiments, the compound is a racemic mixture of (S)- and (R)-isomers. In other embodiments, provided herein is a mixture of compounds wherein individual compounds of the mixture exist predominately in an (S)- or (R)-isomeric configuration. For example, the compound mixture has an (S)-enantiomeric excess of greater than about 55%, about 60%, about 65%, about 70%, about 75%, about 80%, about 85%, about 90%, about 95%, about 96%, about 97%, about 98%, about 99%, about 99.5%, or more. In other embodiments, the compound mixture has an (S)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more.

In other embodiments, the compound mixture has an (R)-enantiomeric purity of greater than about 55%, about 60%, about 65%, about 70%, about 75%, about 80%, about 85%, about 90%, about 95%, about 96%, about 97%, about 98%, about 99%, about 99.5% or more. In some other embodiments, the compound mixture has an (R)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5% or more.

In other embodiments, the compound mixture contains identical chemical entities except for their stereochemical orientations, namely (S)- or (R)-isomers. For example, if a compound disclosed herein has a variable substituent on an asymmetric center, for example, —CH(R)— where R is not hydrogen, then the —CH(R)—is chiral and may have either an (S)- or (R)-stereochemical orientation both of which are represented in the structural presentation. In some embodiments, the structural representation is a mixture of equal amounts of identical chemical entities, that is, a racemic mixture of (S)- and (R)-isomers. In another embodiment, the mixture of enantiomers represented by the structure contains a predominance of one isomer, for example, predominately (S)-isomer or predominately (R)-isomer. For example, the (S)-isomer in the mixture of identical chemical entities are present at about 55%, about 60%, about 65%, about 70%, about 75%, about 80%, about 85%, about 90%, about 95%, about 96%, about 97%, about 98%, about 99%, about 99.5%, or more, relative to the (R)-isomer. In some embodiments, the (S)-isomer in the mixture of identical chemical entities are present at an (S)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5% or more.

In another embodiment, the (R)-isomer in the mixture of identical chemical entities (except for their stereochemical orientations), are present at about 55%, about 60%, about 65%, about 70%, about 75%, about 80%, about 85%, about 90%, about 95%, about 96%, about 97%, about 98%, about 99%, about 99.5%, or more, relative to the (S)-isomer. In some embodiments, the (R)-isomer in the mixture of identical chemical entities (except for their stereochemical orientations), are present at a (R)-enantiomeric excess greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more.

Geometric isomers may occur when a compound contains a double bond or some other feature that gives the molecule a certain amount of structural rigidity. If the compound contains a double bond, the double bond may be E- or Z-configuration. If the compound contains a disubstituted cycloalkyl, the cycloalkyl substituent may have a cis- or trans-configuration.

Conformational isomers (or conformers) are isomers that can differ by rotations about one or more bonds. Rotamers are conformers that differ by rotation about only a single bond.

The term “atropisomer,” as used herein, refers to a structural isomer based on axial or planar chirality resulting from restricted rotation in the molecule.

Optically active (R)- and (S)-isomers may be prepared using chiral synthons or chiral reagents, or resolved using conventional techniques (e.g., separated on chiral SFC or HPLC chromatography columns, such as CHIRALPAK® and CHIRALCEL® columns available from DAICEL Corp. or other equivalent columns, using the appropriate solvent or mixture of solvents to achieve suitable separation).

The compounds of the present disclosure can be isolated in optically active or racemic forms. Optically active forms may be prepared by resolution of racemic forms or by synthesis from optically active starting materials. All processes used to prepare compounds of the present disclosure and intermediates made therein are considered to be part of the present disclosure. When enantiomeric or diastereomeric products are prepared, they may be separated by conventional methods, for example, by chromatography or fractional crystallization.

Depending on the process conditions, the end products of the present disclosure are obtained either in free (neutral) or salt form. Both the free form and the salts of these end products are within the scope of the present disclosure. If so desired, one form of a compound may be converted into another form. A free base or acid may be converted into a salt; a salt may be converted into the free compound or another salt; a mixture of isomeric compounds of the present disclosure may be separated into the individual isomers.

Pharmaceutically acceptable salts are preferred. However, other salts may be useful, e.g., in isolation or purification steps which may be employed during preparation, and thus, are contemplated to be within the scope of the present disclosure.

As used herein, “pharmaceutically acceptable salts” refers to salts derived from suitable inorganic and organic acids and bases that are, within the scope of sound medical judgment, suitable for use in contact with the tissues of humans and lower animals without undue toxicity, irritation, allergic response and the like, and are commensurate with a reasonable benefit/risk ratio.

Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable acid addition salts include, but are not limited to, acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethanedisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate/hydroxymalonate, mandelate, mesylate, methylsulphate, mucate, naphthoate, napsylate, nicotinate, nitrate, octadecanoate, oleate, oxalate, palmitate, pamoate, phenylacetate, phosphate/hydrogen phosphate/dihydrogen phosphate, polygalacturonate, propionate, salicylates, stearate, succinate, sulfamate, sulfosalicylate, tartrate, tosylate, trifluoroacetate and xinafoate salts.

Pharmaceutically acceptable base addition salts can be formed with inorganic and organic bases. Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, or copper; particularly suitable salts include ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like. Examples of organic amines include, but are not limited to, isopropylamine, benzathine, cholinate, diethanolamine, diethylamine, lysine, meglumine, piperazine and tromethamine.

The pharmaceutically acceptable salts of the present disclosure can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Allen, L. V., Jr., ed., Remington: The Science and Practice of Pharmacy, 22nd Edition, Pharmaceutical Press, London, UK (2012), the relevant disclosure of which is hereby incorporated by reference in its entirety.

Compounds of the present disclosure that contain groups capable of acting as donors and/or acceptors for hydrogen bonds may be capable of forming co-crystals with suitable co-crystal formers. These co-crystals may be prepared from compounds of the present disclosure by known co-crystal forming procedures. Such procedures include grinding, heating, co-subliming, co-melting, or contacting in solution compounds of the present disclosure with the co-crystal former under crystallization conditions and isolating co-crystals thereby formed. Suitable co-crystal formers include those described in WO 2004/078163. Hence, the present disclosure further provides co-crystals comprising a compound of the present disclosure and a co-crystal former.

Any formula given herein is also intended to represent unlabeled forms as well as isotopically labeled forms of the compounds. Isotopically labeled forms of the compounds presented herein have the structures of their unlabeled counterparts but are prepared with starting materials or reagents that have been enriched in particular isotopes such that one or more specific atom locations in the structure contain a measurable amount of a non-naturally occurring isotope or an abundance of a natural isotope that exceeds the natural occurrence of that isotope. Examples of isotopes that can be incorporated into compounds of the present disclosure include isotopes of hydrogen, carbon, nitrogen, oxygen, phosphorous, fluorine, chlorine and iodine, such as ²H, ³H, ¹¹C, ¹³C, ¹⁴C, ¹⁵N, ¹⁸F, ³¹P, ³²P, ³⁵S, ³⁶Cl, ¹²³I, ¹²⁴I and ¹²⁵I, respectively. The present disclosure includes various isotopically labeled compounds as defined herein, for example those into which radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies.

Further, substitution with heavier isotopes, particularly deuterium (i.e., ²H or D) may afford certain therapeutic advantages resulting from greater metabolic stability, for example, increased in vivo half-life or reduced dosage requirements or an improvement in therapeutic index. It is understood that deuterium in this context is regarded as a substituent of a compound of the present disclosure. The concentration of such a heavier isotope, specifically deuterium, may be defined by the isotopic enrichment factor. The term “isotopic enrichment factor,” as used herein, means the ratio between the isotopic abundance and the natural abundance of a specified isotope. If a substituent in a compound of this present disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Isotopically labeled compounds of the present disclosure can generally be prepared by conventional techniques known to those skilled in the art or by processes disclosed in the schemes or in the examples and preparations described below (or analogous processes to those described herein below), by substituting an appropriate or readily available isotopically labeled reagent for a non-isotopically labeled reagent otherwise employed. Such compounds have a variety of potential uses, e.g., as standards and reagents in determining the ability of a potential pharmaceutical compound to bind to target proteins or receptors, or for imaging compounds of this disclosure bound to biological receptors in vivo or in vitro.

Where one or more ranges are referred to throughout this specification, each range is understood to encompass each discrete point within the range, including the endpoints describing the range, as if the same were fully set forth herein.

In one aspect, the present disclosure provides compounds of Formula I

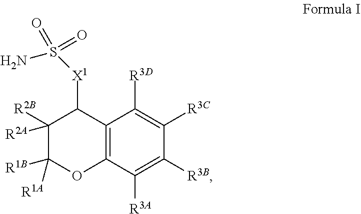

or a pharmaceutically acceptable salt thereof,
wherein:
   X¹ is C(R^{4A})(R^{4B}) or N(R^{4C});
      R^{4A} and R^{4B} are each independently H, (C₁-C₄)alkyl, (C₂-C₄)alkenyl, (C₂-C₄)alkynyl, (C₁-C₄)alkoxy, or (C₃-C₆)cycloalkyl; or
      R^{4A} and R^{4B}, together with the carbon atom to which they are attached, form a (C₃-C₆)cycloalkyl;
      R^{4C} is H, (C₁-C₄)alkyl, (C₂-C₄)alkenyl, (C₂-C₄)alkynyl, or (C₃-C₆)cycloalkyl;
   R^{1A}, R^{1B}, R^{2A}, and R^{2B} are each independently H, halogen, (C₁-C₄)alkyl, (C₂-C₄)alkenyl, (C₂-C₄)alkynyl, (C₁-C₄)alkoxy, (C₁-C₄)haloalkyl, or (C₃-C₆)cycloalkyl;
      or R^{1A} and R^{1B}, together with the carbon atom to which they are attached, form a spiro-(C₃-C₆)cycloalkyl;
      or R^{2A} and R^{2B}, together with the carbon atom to which they are attached, form a spiro-(C₃-C₆)cycloalkyl; and
   R^{3A}, R^{3B}, R^{3C}, and R^{3D} are each independently H, halogen, hydroxyl, (C₁-C₄)alkyl, (C₂-C₄)alkenyl, (C₂-C₄)alkynyl, (C₁-C₄)alkoxy, (C₁-C₄)haloalkyl, acyl, —CN, or (C₃-C₆)cycloalkyl;
   with the provisos that:
   when R^{1A}, R^{1B}, R^{2A}, R^{2B}, R^{3A}, R^{3B}, R^{3C}, and R^{3D} are each hydrogen, then (i) at least one of R^{4A} and R^{4B} is a group other than hydrogen or (ii) R^{4C} is a group other than hydrogen; and
   when X¹ is N(R^{4C}) and only one of R^{1A}, R^{1B}, R^{2A}, R^{2B}, R^{3A}, R^{3B}, R^{3C}, R^{3D}, and R^{4C} is a group other than hydrogen, then (i) R^{1A} is not methyl; (ii) R^{1B} is not methyl; and (iii) R^{3C} is not methoxy or ethoxy.

In another aspect, provided herein is a compound of Formula I

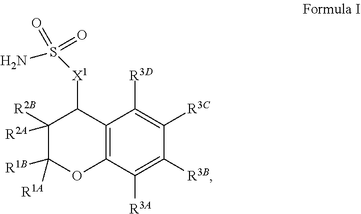

or a pharmaceutically acceptable salt thereof,
wherein:
   X¹ is C(R^{4A})(R^{4B}) or N(R^{4C});
      R^{4A} and R^{4B} are each independently H, (C₁-C₄)alkyl, (C₂-C₄)alkenyl, (C₂-C₄)alkynyl, (C₁-C₄)alkoxy, or (C₃-C₆)cycloalkyl; or
      R^{4A} and R^{4B}, together with the carbon atom to which they are attached, form a (C₃-C₆)cycloalkyl;
      R^{4C} is H, (C₁-C₄)alkyl, (C₂-C₄)alkenyl, (C₂-C₄)alkynyl, or (C₃-C₆)cycloalkyl;
   R^{1A}, R^{1B}, R^{2A}, and R^{2B} are each independently H, halogen, (C₁-C₄)alkyl, (C₂-C₄)alkenyl, (C₂-C₄)alkynyl, (C₁-C₄)alkoxy, (C₁-C₄)haloalkyl, or (C₃-C₆)cycloalkyl;
      or R^{1A} and R^{1B}, together with the carbon atom to which they are attached, form a spiro-(C₃-C₆)cycloalkyl;
      or R^{2A} and R^{2B}, together with the carbon atom to which they are attached, form a spiro-(C₃-C₆)cycloalkyl; and
   R^{3A}, R^{3B}, R^{3C}, and R^{3D} are each independently H, halogen, hydroxyl, (C₁-C₄)alkyl, (C₂-C₄)alkenyl, (C₂-C₄)alkynyl, (C₁-C₄)alkoxy, (C₁-C₄)haloalkyl, acyl, —CN, or (C₃-C₆)cycloalkyl;
with the provisos that:
   when R^{1A}, R^{1B}, R^{2A}, R^{2B}, R^{3A}, R^{3B}, R^{3C}, and R^{3D} are each hydrogen, then (i) at least one of R^{4A} and R^{4B} is a group other than hydrogen or (ii) R^{4C} is a group other than hydrogen; and
   the compound is not
N-(6-ethoxy-3,4-dihydro-2H-1-benzopyran-4-yl)-sulfamide;
N-[(4S)-3,4-dihydro-6-methoxy-2H-1-benzopyran-4-yl]-sulfamide;
N-[(2R,4R)-3,4-dihydro-2-methyl-2H-1-benzopyran-4-yl]-sulfamide; or
N-[(2S,4S)-3,4-dihydro-2-methyl-2H-1-benzopyran-4-yl]-sulfamide.

In another aspect, provided herein is a compound of Formula I(A)

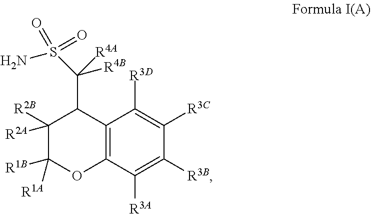

or a pharmaceutically acceptable salt thereof,
wherein:
   R^{4A} and R^{4B} are each independently H, (C₁-C₄)alkyl, (C₂-C₄)alkenyl, (C₂-C₄)alkynyl, (C₁-C₄)alkoxy, or (C₃-C₆)cycloalkyl;
   or R^{4A} and R^{4B}, together with the carbon atom to which they are attached, form a (C₃-C₆)cycloalkyl;
   R^{1A}, R^{1B}, R^{2A}, and R^{2B} are each independently H, halogen, (C₁-C₄)alkyl, (C₂-C₄)alkenyl, (C₂-C₄)alkynyl, (C₁-C₄)alkoxy, (C₁-C₄)haloalkyl, or (C₃-C₆)cycloalkyl;
      or R^{1A} and R^{1B}, together with the carbon atom to which they are attached, form a spiro-(C₃-C₆)cycloalkyl;
      or R^{2A} and R^{2B}, together with the carbon atom to which they are attached, form a spiro-(C₃-C₆)cycloalkyl; and
   R^{3A}, R^{3B}, R^{3C}, and R^{3D} are each independently H, halogen, hydroxyl, (C₁-C₄)alkyl, (C₂-C₄)alkenyl, (C₂-C₄)alkynyl, (C₁-C₄)alkoxy, (C₁-C₄)haloalkyl, acyl, —CN, or (C₃-C₆)cycloalkyl;
   with the proviso that:
   when R^{1A}, R^{1B}, R^{2A}, R^{2B}, R^{3A}, R^{3B}, R^{3C}, and R^{3D} are each hydrogen, then at least one of R^{4A} and R^{4B} is a group other than hydrogen.

In another aspect, provided herein is a compound of Formula I(B)

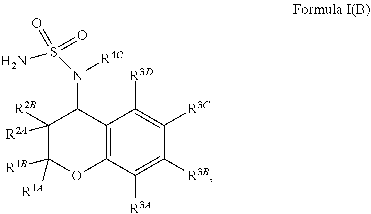

or a pharmaceutically acceptable salt thereof,
wherein:
   R^{4C} is H, (C₁-C₄)alkyl, (C₂-C₄)alkenyl, (C₂-C₄)alkynyl, or (C₃-C₆)cycloalkyl;
   R^{1A}, R^{1B}, R^{2A}, and R^{2B} are each independently H, halogen, (C₁-C₄)alkyl, (C₂-C₄)alkenyl, (C₂-C₄)alkynyl, (C₁-C₄)alkoxy, (C₁-C₄)haloalkyl, or (C₃-C₆)cycloalkyl;
      or R^{1A} and R^{1B}, together with the carbon atom to which they are attached, form a spiro-(C₃-C₆)cycloalkyl;
      or R^{2A} and R^{2B}, together with the carbon atom to which they are attached, form a spiro-(C₃-C₆)cycloalkyl; and
   R^{3A}, R^{3B}, R^{3C}, and R^{3D} are each independently H, halogen, hydroxyl, (C₁-C₄)alkyl, (C₂-C₄)alkenyl, (C₂-C₄)alkynyl, (C₁-C₄)alkoxy, (C₁-C₄)haloalkyl, acyl, —CN, or (C₃-C₆)cycloalkyl;
   with the provisos that:
   when R^{1A}, R^{1B}, R^{2A}, R^{2B}, R^{3A}, R^{3B}, R^{3C}, and R^{3D} are each hydrogen, then R^{4C} is a group other than hydrogen; and
   when only one of R^{1A}, R^{1B}, R^{2A}, R^{2B}, R^{3A}, R^{3B}, R^{3C}, R^{3D}, and R^{4C} is a group other than hydrogen, then (i) R^{1A} is not methyl; (ii) R^{1B} is not methyl; and (iii) R^{3C} is not methoxy or ethoxy.

In some embodiments, one of R^{1A}, R^{1B}, R^{2A}, R^{2B}, R^{3A}, R^{3B}, R^{3C}, R^{3D} is a group other than hydrogen. In some embodiments, one and only one of R^{1A}, R^{1B}, R^{2A}, R^{2B}, R^{3A}, R^{3B}, R^{3C}, R^{3D} is a group other than hydrogen.

In some embodiments, one of R^{1A}, R^{1B}, R^{2A}, R^{2B}, R^{3A}, R^{3B}, R^{3C}, R^{3D}, and R^{4C} is a group other than hydrogen. In some embodiments, one and only one of R^{1A}, R^{1B}, R^{2A}, R^{2B}, R^{3A}, R^{3B}, R^{3C}, R^{3D}, and R^{4C} is a group other than hydrogen.

In some embodiments, one of R^{1A}, R^{1B}, R^{2A}, R^{2B}, R^{3A}, R^{3B}, R^{3C}, R^{3D}, R^{4A}, and R^{4B} is a group other than hydrogen. In some embodiments, one and only one of R^{1A}, R^{1B}, R^{2A}, R^{2B}, R^{3A}, R^{3B}, R^{3C}, R^{3D}, R^{4A}, and R^{4B} is a group other than hydrogen.

In some embodiments, one of R^{1A}, R^{1B}, R^{2A}, R^{2B}, R^{3A}, R^{3B}, R^{3C}, R^{3D}, R^{4A}, R^{4B}, and R^{4C} is a group other than hydrogen. In some embodiments, one and only one of R^{1A}, R^{1B}, R^{2A}, R^{2B}, R^{3A}, R^{3B}, R^{3C}, R^{3D}, R^{4A}, R^{4B}, and R^{4C} is a group other than hydrogen.

In some embodiments, X¹ is C(R^{4A})(R^{4B}). In some embodiments, X¹ is N(R^{4C}). In some embodiments, X¹ is CH₂ or NH. In some embodiments, X¹ is CH₂. In some embodiments, X¹ is NH.

In some embodiments, R^{4A}, R^{4B}, and R^{4C} are each independently H, (C₁-C₄)alkyl, (C₂-C₄)alkenyl, (C₂-C₄)alkynyl, (C₁-C₄)alkoxy, or (C₃-C₆)cycloalkyl.

In some embodiments, R^{4A}, R^{4B}, and R^{4C} are each independently H or (C₁-C₄)alkyl. In some embodiments, R^{4A}, R^{4B}, and R^{4C} are each independently H, methyl, or ethyl.

In some embodiments, R^{4A} and R^{4B} are each independently H, (C₁-C₄)alkyl, (C₂-C₄)alkenyl, (C₂-C₄)alkynyl, (C₁-C₄)alkoxy, or (C₃-C₆)cycloalkyl. In some embodiments, R^{4A} and R^{4B} are each independently H, (C₁-C₄)alkyl, (C₂-C₄)alkenyl, (C₂-C₄)alkynyl, (C₁-C₄)alkoxy. In some embodiments, R^{4A} and R^{4B} are each independently (C₁-C₄)alkyl, (C₂-C₄)alkenyl, (C₂-C₄)alkynyl, (C₁-C₄)alkoxy, or (C₃-C₆)cycloalkyl.

In some embodiments, R^{4A} and R^{4B} are each independently H or (C₁-C₄)alkyl. In some embodiments, R^{4A} and R^{4B} are each independently H, methyl, or ethyl. In some embodiments, R^{4A} is H, and R^{4B} is H, methyl, or ethyl. In some embodiments, R^{4A} is H, and R^{4B} is methyl or ethyl. In some embodiments, R^{4A} and R^{4B} are each H. In some embodiments, R^{4A} is H, and R^{4B} is (C₁-C₄)alkyl. In some embodiments, R^{4A} and R^{4B} are each (C₁-C₄)alkyl. In some embodiments, R^{4A} and R^{4B} are each methyl.

In some embodiments, R^{4A} is H, (C₁-C₄)alkyl, (C₂-C₄)alkenyl, (C₂-C₄)alkynyl, (C₁-C₄)alkoxy, or (C₃-C₆)cycloalkyl. In some embodiments, R^{4A} is H or (C₁-C₄)alkyl. In some embodiments, R^{4A} is (C₁-C₄)alkyl, (C₂-C₄)alkenyl, (C₂-C₄)alkynyl, (C₁-C₄)alkoxy, or (C₃-C₆)cycloalkyl. In some embodiments, R^{4A} is (C₁-C₄)alkyl. In some embodiments, R^{4A} is H, methyl, or ethyl. In some embodiments, R^{4A} is H. In some embodiments, R^{4A} is methyl or ethyl. In some embodiments, R^{4A} is methyl. In some embodiments, R^{4A} is ethyl.

In some embodiments, R^{4B} is H, (C₁-C₄)alkyl, (C₂-C₄)alkenyl, (C₂-C₄)alkynyl, (C₁-C₄)alkoxy, or (C₃-C₆)cycloalkyl. In some embodiments, R^{4B} is H or (C₁-C₄)alkyl. In some embodiments, R^{4B} is (C₁-C₄)alkyl, (C₂-C₄)alkenyl, (C₂-C₄)alkynyl, (C₁-C₄)alkoxy, or (C₃-C₆)cycloalkyl. In some embodiments, R^{4B} is (C₁-C₄)alkyl. In some embodiments, R^{4B} is H, methyl, or ethyl. In some embodiments, R^{4B} is H. In some embodiments, R^{4B} is methyl or ethyl. In some embodiments, R^{4B} is methyl. In some embodiments, R^{4B} is ethyl.

In some embodiments, R^{4A} and R^{4B}, together with the carbon atom to which they are attached, form a (C₃-C₆)cycloalkyl. In some embodiments, R^{4A} and R^{4B}, together with the carbon atom to which they are attached, form a cyclopropyl group.

In some embodiments, R^{4C} is H, (C₁-C₄)alkyl, (C₂-C₄)alkenyl, (C₂-C₄)alkynyl, or (C₃-C₆)cycloalkyl. In some embodiments, R^{4C} is H or (C₁-C₄)alkyl. In some embodiments, R^{4C} is H or methyl. In some embodiments, R^{4C} is H. In some embodiments, R^{4C} is H.

In some embodiments, R^{1A}, R^{1B}, R^{2A}, and R^{2B} are each independently H, halogen, (C₁-C₄)alkyl, (C₂-C₄)alkenyl, (C₂-C₄)alkynyl, (C₁-C₄)alkoxy, (C₁-C₄)haloalkyl, or (C₃-C₆)cycloalkyl. In some embodiments, R^{1A}, R^{1B}, R^{2A}, and R^{2B} are each independently H, halogen, (C₁-C₄)alkyl, (C₂-C₄)alkenyl, (C₂-C₄)alkynyl, (C₁-C₄)alkoxy, or (C₁-C₄)haloalkyl. In some embodiments, R^{1A}, R^{1B}, R^{2A}, and R^{2B} are each independently H or (C₁-C₄)alkyl. In some embodiments, R^{1A}, R^{1B}, R^{2A}, and R^{2B} are each independently H, methyl, or ethyl.

In some embodiments, R^{1A} and R^{1B} are each independently H, halogen, (C₁-C₄)alkyl, (C₂-C₄)alkenyl, (C₂-C₄)alkynyl, (C₁-C₄)alkoxy, (C₁-C₄)haloalkyl, or (C₃-C₆)cycloalkyl. In some embodiments, R^{1A} and R^{1B} are each independently H, halogen, (C₁-C₄)alkyl, (C₂-C₄)alkenyl, (C₂-C₄)alkynyl, (C₁-C₄)alkoxy, or (C₁-C₄)haloalkyl. In some embodiments, R^{1A} and R^{1B} are each independently H or (C₁-C₄)alkyl. In some embodiments, R^{1A} and R^{1B} are each independently H, methyl, or ethyl. In some embodiments, R^{1A} is H, and R^{1B} is H, methyl, or ethyl. In some embodiments, R^{1A} is H, and R^{1B} is (C₁-C₄)alkyl. In some embodiments, R^{1A} is H, and R^{1B} is methyl or ethyl. In some embodiments, R^{1A} and R^{1B} are each methyl.

In some embodiments, R^{1A} is H, methyl, or ethyl. In some embodiments, R^{1A} is H. In some embodiments, R^{1A} is methyl or ethyl.

In some embodiments, R^{1B} is H, methyl, or ethyl. In some embodiments, R^{1B} is H. In some embodiments, R^{1B} is methyl or ethyl.

In some embodiments, R^{1A} and R^{1B}, together with the carbon atom to which they are attached, form a spiro-(C₃-C₆)cycloalkyl. In some embodiments, R^{1A} and R^{1B}, together with the carbon atom to which they are attached, form a spiro-cyclopropyl group or a spiro-cyclobutyl group. In some embodiments, R^{1A} and R^{1B}, together with the carbon atom to which they are attached, form a spiro-cyclopropyl group. In some embodiments, R^{1A} and R^{1B}, together with the carbon atom to which they are attached, form a spiro-butyl group.

In some embodiments, R^{2A} and R^{2B} are each independently H, halogen, (C₁-C₄)alkyl, (C₂-C₄)alkenyl, (C₂-C₄)alkynyl, (C₁-C₄)alkoxy, (C₁-C₄)haloalkyl, or (C₃-C₆)cycloalkyl. In some embodiments, R^{2A} and R^{2B} are each independently H, halogen, (C₁-C₄)alkyl, (C₂-C₄)alkenyl, (C₂-C₄)alkynyl, (C₁-C₄)alkoxy, or (C₁-C₄)haloalkyl. In some embodiments, R^{2A} and R^{2B} are each independently H or (C₁-C₄)alkyl. In some embodiments, R^{2A} and R^{2B} are each independently H, methyl, or ethyl. In some embodiments, R^{2A} is H, and R^{2B} is H, methyl, or ethyl. In some embodiments, R^{2A} is H, and R^{2B} is (C₁-C₄)alkyl. In some embodiments, R^{2A} is H, and R^{2B} is methyl or ethyl. In some embodiments, R^{2A} and R^{2B} are each methyl.

In some embodiments, R^{2A} is H, methyl, or ethyl. In some embodiments, R^{2A} is H. In some embodiments, R^{2A} is methyl or ethyl.

In some embodiments, R^{2B} is H, methyl, or ethyl. In some embodiments, R^{2B} is H. In some embodiments, R^{2B} is methyl or ethyl.

In some embodiments, R^{3A}, R^{3B}, R^{3C}, and R^{3D} are each independently H, halogen, (C₁-C₄)alkyl, (C₁-C₄)haloalkyl, or —CN. In some embodiments, R^{3A}, R^{3B}, R^{3C}, and R^{3D} are each independently H, F, Cl, methyl, CF₃, or —CN.

In some embodiments, R^{3A} is H or halogen. In some embodiments, R^{3A} is H or F. In some embodiments, R^{3A} is H. In some embodiments, R^{3A} is F. In some embodiments, R^{3A} is halogen.

In some embodiments, R^{3B} is H, halogen, (C₁-C₄)alkyl, (C₁-C₄)haloalkyl, or —CN. In some embodiments, R^{3B} is halogen, (C₁-C₄)alkyl, (C₁-C₄)haloalkyl, or —CN. In some embodiments, R^{3B} is H, F, Cl, methyl, CF₃, or —CN. In some embodiments, R^{3B} is F, Cl, methyl, CF₃, or —CN. In some embodiments, R^{3B} is F.

In some embodiments, R^{3C} is H or halogen. In some embodiments, R^{3C} is H or F. In some embodiments, R^{3C} is H. In some embodiments, R^{3C} is F. In some embodiments, R^{3C} is halogen.

In some embodiments, R^{3D} is H or halogen. In some embodiments, R^{3D} is H or F. In some embodiments, R^{3D} is H. In some embodiments, R^{3D} is F. In some embodiments, R^{3D} is halogen.

In some embodiments, R^{3A}, R^{3C}, and R^{3D} are each H, and R^{3B} is H, halogen, (C₁-C₄)alkyl, (C₁-C₄)haloalkyl, or —CN. In some embodiments, R^{3A}, R^{3C}, and R^{3D} are each H, and R^{3B} is halogen, (C₁-C₄)alkyl, (C₁-C₄)haloalkyl, or —CN.

In some embodiments, R^{3A}, R^{3C}, and R^{3D} are each H, and R^{3B} is H, F, Cl, methyl, CF₃, or —CN. In some embodiments, R^{3A}, R^{3C}, and R^{3D} are each H, and R^{3B} is F, Cl, methyl, CF₃, or —CN.

In some embodiments, provided herein is a compound of Formula I, or a pharmaceutically acceptable salt thereof, wherein:
X¹ is C(R^{4A})(R^{4B}) or N(R^{4C});
   R^{4A}, R^{4B}, and R^{4C} are each independently H or (C₁-C₄)alkyl;
   or R^{4A} and R^{4B}, together with the carbon atom to which they are attached, form a (C₃-C₆)cycloalkyl;
R^{1A}, R^{1B}, R^{2A}, and R^{2B} are each independently H or (C₁-C₄)alkyl;
   or R^{1A} and R^{1B}, together with the carbon atom to which they are attached, form a spiro-(C₃-C₆)cycloalkyl; and
R^{3A}, R^{3B}, R^{3C}, and R^{3D} are each independently H, halogen, (C₁-C₄)alkyl, (C₁-C₄)haloalkyl, or —CN;
with the provisos that:
when R^{1A}, R^{1B}, R^{2A}, R^{2B}, R^{3A}, R^{3B}, R^{3C}, and R^{3D} are each hydrogen, then (i) at least one of R^{4A} and R^{4B} is a group other than hydrogen or (ii) R^{4C} is a group other than hydrogen; and
when X¹ is N(R^{4C}) and only one of R^{1A}, R^{1B}, R^{2A}, R^{2B}, R^{3A}, R^{3B}, R^{3C}, R^{3D}, and R^{4C} is a group other than hydrogen, then (i) R^{1A} is not methyl; and (ii) R^{1B} is not methyl.

In some embodiments, X¹ is C(R^{4A})(R^{4B}) and R^{4A} and R^{4B} are each independently H or (C₁-C₄)alkyl; or R^{4A} and R^{4B} are each independently H, methyl, or ethyl; or R^{4A} is H, and R^{4B} is H, methyl, or ethyl; or R^{4A} is H, and R^{4B} is methyl or ethyl; or R^{4A} and R^{4B} are each H; or R^{4A} and R^{4B}, together with the carbon atom to which they are attached, form a (C₃-C₆)cycloalkyl; or R^{4A} and R^{4B}, together with the carbon atom to which they are attached, form a cyclopropyl. In some embodiments, R^{1A} and R^{1B} are each independently H or (C₁-C₄)alkyl; or R^{1A} and R^{1B} are each independently H, methyl, or ethyl; or R^{1A} is H, and R^{1B} is H, methyl, or ethyl; or R^{1A} and R^{1B} are each methyl; or R^{1A} and R^{1B} are each H; or R^{1A} and R^{1B}, together with the carbon atom to which they are attached, form a spiro-(C₃-C₆)cycloalkyl; or R^{1A} and R^{1B}, together with the carbon atom to which they are attached, form a spiro-cyclopropyl or a spiro-cyclobutyl. In some embodiments, R^{2A} and R^{2B} are each independently H or (C₁-C₄)alkyl; or R^{2A} and R^{2B} are each independently H, methyl, or ethyl; or R^{2A} is H, and R^{2B} is H, methyl, or ethyl; or R^{2A} and R^{2B} are each methyl; or R^{2A} and R^{2B} are each H. In some embodiments, R^{3A}, R^{3B}, R^{3C}, and R^{3D} are each independently H, halogen, (C₁-C₄)alkyl, (C₁-C₄)haloalkyl, or —CN; or R^{3A}, R^{3B}, R^{3C}, and R^{3D} are each independently H, F, Cl, methyl, CF₃, or —CN; or R^{3A}, R^{3C}, and R^{3D} are each H, and R^{3B} is H, halogen, (C₁-C₄)alkyl, (C₁-C₄)haloalkyl, or —CN; or R^{3A}, R^{3C}, and R^{3D} are each H, and R^{3B} is H, F, Cl, methyl, CF₃, or —CN. When R^{1A}, R^{1B}, R^{2A}, R^{2B}, R^{3A}, R^{3B}, R^{3C}, and R^{3D} are each hydrogen, then at least one of R^{4A} and R^{4B} is a group other than hydrogen. This list of substituents also applies to other formulae disclosed herein, including Formulae I(A), I(B), II(A), II(B), III, III(A), III(B), IV, and IV(A), when such substituents are present in the formula.

In some embodiments, X¹ is N(R^{4C}) and R^{4C} is H or (C₁-C₄)alkyl; or R^{4C} is H; or R^{4C} is methyl. In some embodiments, R^{1A} and R^{1B} are each independently H or (C₁-C₄)alkyl; or RIA and R^{1B} are each independently H, methyl, or ethyl; or R^{1A} is H, and R^{1B} is H, methyl, or ethyl; or R^{1A} and R^{1B} are each methyl; or R^{1A} and R^{1B} are each H; or R^{1A} and R^{1B}, together with the carbon atom to which they are attached, form a spiro-(C₃-C₆)cycloalkyl; or R^{1A} and R^{1B}, together with the carbon atom to which they are attached, form a spiro-cyclopropyl or a spiro-cyclobutyl. In some embodiments, R^{2A} and R^{2B} are each independently H or (C₁-C₄)alkyl; or R^{2A} and R^{2B} are each independently H, methyl, or ethyl; or R^{2A} is H, and R^{2B} is H, methyl, or ethyl; or R^{2A} and R^{2B} are each methyl; or R^{2A} and R^{2B} are each H. In some embodiments, R^{3A}, R^{3B}, R^{3C}, and R^{3D} are each independently H, halogen, (C₁-C₄)alkyl, (C₁-C₄)haloalkyl, or —CN; or R^{3A}, R^{3B}, R^{3C}, and R^{3D} are each independently H, F, Cl, methyl, CF₃, or —CN; or R^{3A}, R^{3C}, and R^{3D} are each H, and R^{3B} is H, halogen, (C₁-C₄)alkyl, (C₁-C₄)haloalkyl, or —CN; or R^{3A}, R^{3C}, and R^{3D} are each H, and R^{3B} is H, F, Cl, methyl, CF₃, or —CN. When R^{1A}, R^{1B}, R^{2A}, R^{2B}, R^{3A}, R^{3B}, R^{3C}, and R^{3D} are each hydrogen, then R^{4C} is a group other than hydrogen; and when only one of R^{1A}, R^{1B}, R^{2A}, R^{2B}, R^{3A}, R^{3B}, R^{3C}, R^{3D}, and R^{4C} is a group other than hydrogen, then (i) R^{1A} is not methyl; and (ii) R^{1B} is not methyl. This list of substituents also applies to other formulae disclosed herein, including Formulae I(A), I(B), II(A), II(B), III, III(A), III(B), IV, and IV(A), when such substituents are present in the formula.

In some embodiments, provided herein is a compound of Formula II(A)

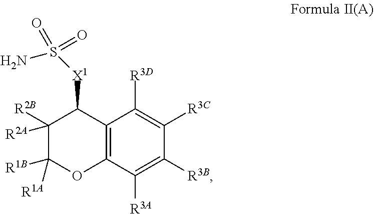

or a pharmaceutically acceptable salt thereof, wherein X¹, R^{1A}, R^{1B}, R^{2A}, R^{2B}, R^{3A}, R^{3B}, R^{3C}, and R^{3D} are as defined herein.

In some embodiments, provided herein is a compound of Formula II(B)

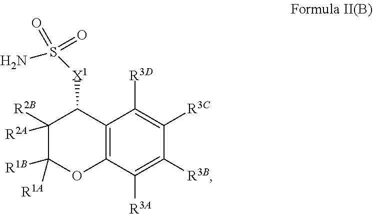

or a pharmaceutically acceptable salt thereof, wherein X¹, R^{1A}, R^{1B}, R^{2A}, R^{2B}, R^{3A}, R^{3B}, R^{3C}, and R^{3D} are as defined herein.

In some embodiments, provided herein is a compound of Formula III

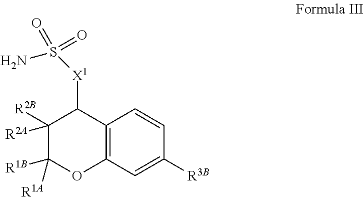

or a pharmaceutically acceptable salt thereof, wherein X¹, R^{1A}, R^{1B}, R^{2A}, R^{2B}, and R^{3B} are as defined herein.

In some embodiments, provided herein is a compound of Formula III(A)

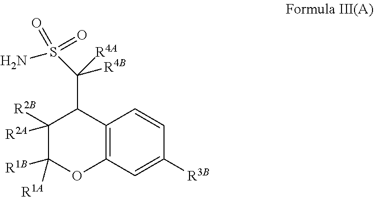

or a pharmaceutically acceptable salt thereof, wherein R^{1A}, R^{1B}, R^{2A}, R^{2B}, R^{3B}, R^{4A}, and R^{4B} are as defined herein.

In some embodiments, provided herein is a compound of Formula III(B)

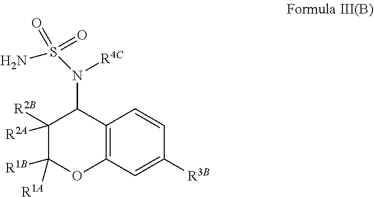

or a pharmaceutically acceptable salt thereof, wherein R^{1A}, R^{1B}, R^{2A}, R^{2B}, R^{3B}, and R^{4C} are as defined herein.

In some embodiments, provided herein is a compound of Formula IV

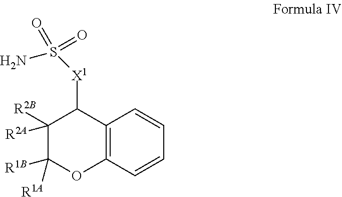

or a pharmaceutically acceptable salt thereof, wherein X¹, R^{1A}, R^{1B}, R^{2A}, and R^{2B} are as defined herein.

In some embodiments, provided herein is a compound of Formula IV(A)

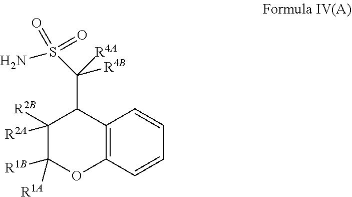

or a pharmaceutically acceptable salt thereof, wherein R^{1A}, R^{1B}, R^{2A}, R^{2B}, R^{4A}, and R^{4B} areas defined herein.

In one embodiment, provided is a compound according to Formula I wherein said compound is selected from the following Table 1: Table 1. Compounds

TABLE 1
Compounds

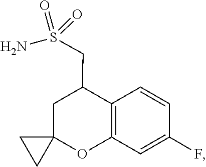 [SMILES: NS(=O)(=O)CC1CC2(CC2)Oc2cc(F)ccc21]

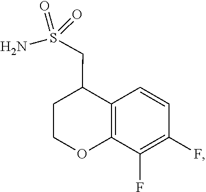 [SMILES: NS(=O)(=O)CC1CCOc2c1ccc(F)c2F]

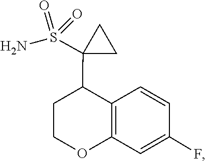 [SMILES: NS(=O)(=O)C1(C2CCOc3cc(F)ccc32)CC1]

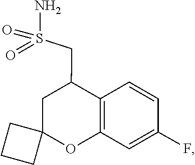 [SMILES: NS(=O)(=O)CC1CC2(CCC2)Oc2cc(F)ccc21]

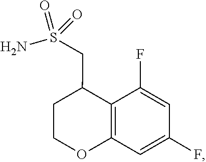 [SMILES: NS(=O)(=O)CC1CCOc2cc(F)cc(F)c21]

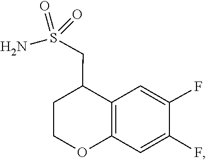 [SMILES: NS(=O)(=O)CC1CCOc2cc(F)c(F)cc21]

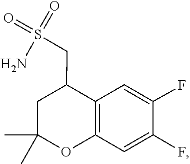 [SMILES: CC1(C)CC(CS(N)(=O)=O)c2cc(F)c(F)cc2O1]

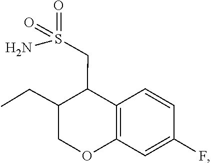 [SMILES: CCC1COc2cc(F)ccc2C1CS(N)(=O)=O]

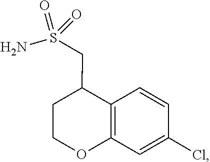 [SMILES: NS(=O)(=O)CC1CCOc2cc(Cl)ccc21]

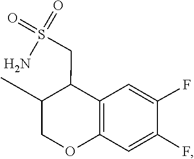 [SMILES: CC1COc2cc(F)c(F)cc2C1CS(N)(=O)=O]

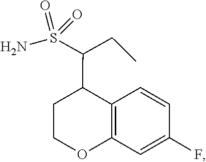 [SMILES: CCC(C1CCOc2cc(F)ccc21)S(N)(=O)=O]

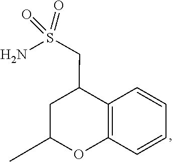 [SMILES: CC1CC(CS(N)(=O)=O)c2ccccc2O1]

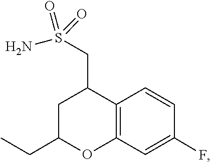 [SMILES: CCC1CC(CS(N)(=O)=O)c2ccc(F)cc2O1]

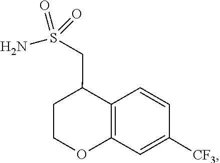 [SMILES: NS(=O)(=O)CC1CCOc2cc(C(F)(F)F)ccc21]

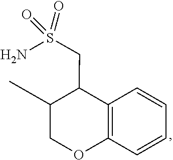 [SMILES: CC1COc2ccccc2C1CS(N)(=O)=O]

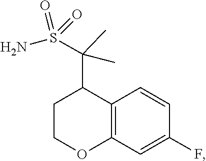 [SMILES: CC(C)(C1CCOc2cc(F)ccc21)S(N)(=O)=O]

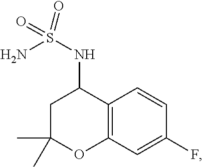 [SMILES: CC1(C)CC(NS(N)(=O)=O)c2ccc(F)cc2O1]

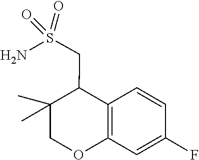 [SMILES: CC1(C)COc2cc(F)ccc2C1CS(N)(=O)=O]

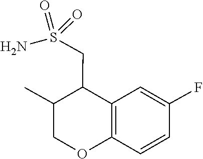 [SMILES: CC1COc2ccc(F)cc2C1CS(N)(=O)=O]

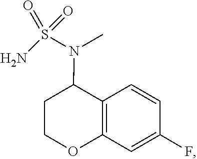 [SMILES: CN(C1CCOc2cc(F)ccc21)S(N)(=O)=O]

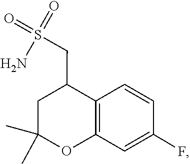 [SMILES: CC1(C)CC(CS(N)(=O)=O)c2ccc(F)cc2O1]

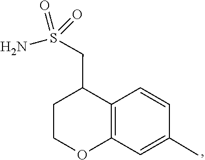 [SMILES: Cc1ccc2c(c1)OCCC2CS(N)(=O)=O]

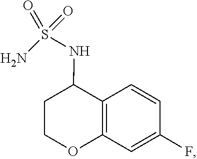 [SMILES: NS(=O)(=O)NC1CCOc2cc(F)ccc21]

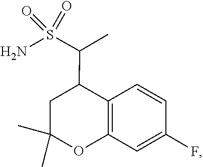 [SMILES: CC(C1CC(C)(C)Oc2cc(F)ccc21)S(N)(=O)=O]

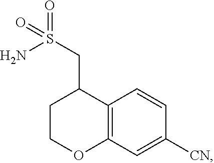 [SMILES: N#Cc1ccc2c(c1)OCCC2CS(N)(=O)=O]

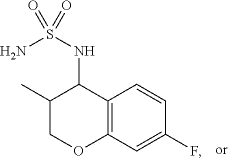 [SMILES: CC1COc2cc(F)ccc2C1NS(N)(=O)=O ; "or"]

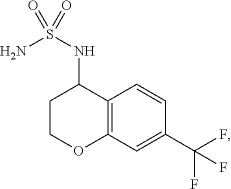 [SMILES: NS(=O)(=O)NC1CCOc2cc(C(F)(F)F)ccc21]

or a pharmaceutically acceptable salt of any of the aforementioned.

In another embodiment, provided are the stereoisomers of the compounds in Table 1 or a pharmaceutically acceptable salt thereof. In some embodiments, the stereoisomers of the compounds in Table 1 may be the enantiomers of the compounds or a pharmaceutically acceptable salt thereof. In some embodiments, the stereoisomers of the compounds in Table 1 may be the diastereomers of the compounds or a pharmaceutically acceptable salt thereof. In some embodiments, provided is a compound of Table 1 that includes a mix of the compound enantiomers (i.e., two stereoisomers of the compound). In some embodiments, provided is a compound of Table 1 that includes a mix of the compound enantiomers and diastereomers (i.e., two, three, or four stereoisomers of the compound). Alternatively, in some embodiments, provided is a compound of Table 1 that includes one stereoisomer of the compound. For example, provided is a compound according to Formula I, wherein said compound is a stereoisomer of a compound in Table 1 as provided in Table 1A.

TABLE 1A
Compounds

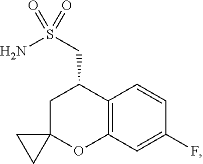 [SMILES: NS(=O)(=O)C[C@H]1CC2(CC2)Oc2cc(F)ccc21]

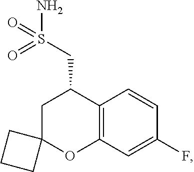 [SMILES: NS(=O)(=O)C[C@H]1CC2(CCC2)Oc2cc(F)ccc21]

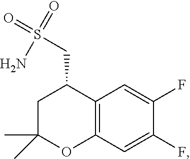 [SMILES: CC1(C)C[C@H](CS(N)(=O)=O)c2cc(F)c(F)cc2O1]

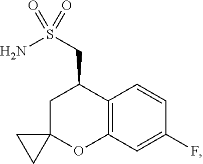 [SMILES: NS(=O)(=O)C[C@@H]1CC2(CC2)Oc2cc(F)ccc21]

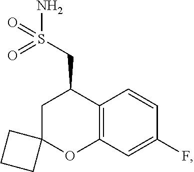 [SMILES: NS(=O)(=O)C[C@@H]1CC2(CCC2)Oc2cc(F)ccc21]

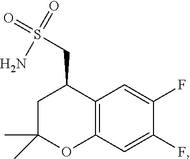 [SMILES: CC1(C)C[C@@H](CS(N)(=O)=O)c2cc(F)c(F)cc2O1]

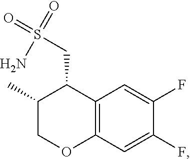 [SMILES: C[C@H]1COc2cc(F)c(F)cc2[C@H]1CS(N)(=O)=O]

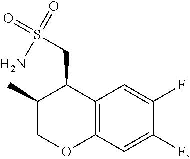 [SMILES: C[C@@H]1COc2cc(F)c(F)cc2[C@@H]1CS(N)(=O)=O]

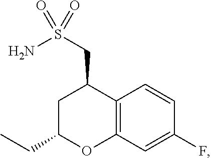 [SMILES: CC[C@@H]1C[C@@H](CS(N)(=O)=O)c2ccc(F)cc2O1]

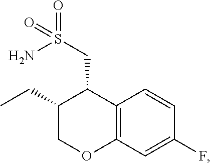 [SMILES: CC[C@H]1COc2cc(F)ccc2[C@H]1CS(N)(=O)=O]

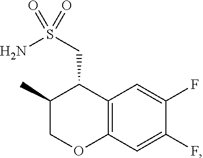 [SMILES: C[C@@H]1COc2cc(F)c(F)cc2[C@H]1CS(N)(=O)=O]

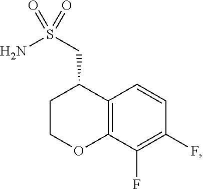 [SMILES: NS(=O)(=O)C[C@H]1CCOc2c1ccc(F)c2F]

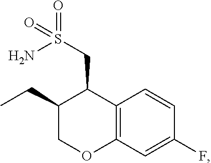 [SMILES: CC[C@@H]1COc2cc(F)ccc2[C@@H]1CS(N)(=O)=O]

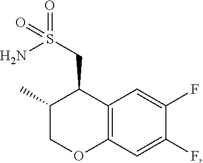 [SMILES: C[C@H]1COc2cc(F)c(F)cc2[C@@H]1CS(N)(=O)=O]

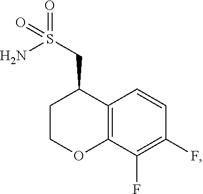 [SMILES: NS(=O)(=O)C[C@@H]1CCOc2c1ccc(F)c2F]

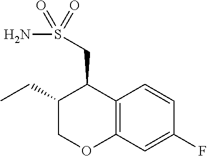 [SMILES: CC[C@H]1COc2cc(F)ccc2[C@@H]1CS(N)(=O)=O]

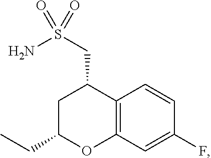 [SMILES: CC[C@@H]1C[C@H](CS(N)(=O)=O)c2ccc(F)cc2O1]

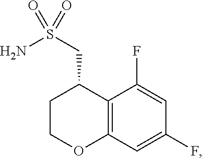 [SMILES: NS(=O)(=O)C[C@H]1CCOc2cc(F)cc(F)c21]

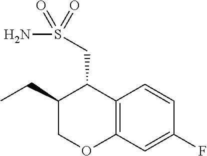 [SMILES: CC[C@@H]1COc2cc(F)ccc2[C@H]1CS(N)(=O)=O]

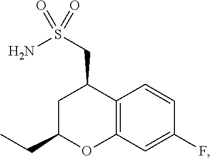 [SMILES: CC[C@H]1C[C@@H](CS(N)(=O)=O)c2ccc(F)cc2O1]

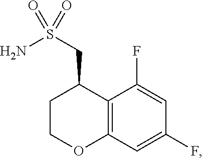 [SMILES: NS(=O)(=O)C[C@@H]1CCOc2cc(F)cc(F)c21]

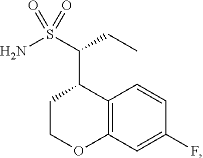 [SMILES: CC[C@H]([C@H]1CCOc2cc(F)ccc21)S(N)(=O)=O]

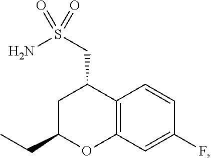 [SMILES: CC[C@H]1C[C@H](CS(N)(=O)=O)c2ccc(F)cc2O1]

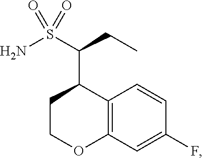 [SMILES: CC[C@@H]([C@@H]1CCOc2cc(F)ccc21)S(N)(=O)=O]

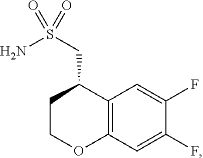

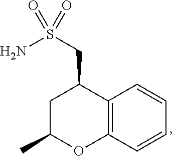 [SMILES: C[C@H]1C[C@@H](CS(N)(=O)=O)c2ccccc2O1]

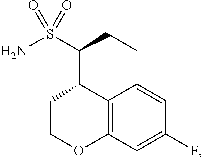 [SMILES: CC[C@@H]([C@H]1CCOc2cc(F)ccc21)S(N)(=O)=O]

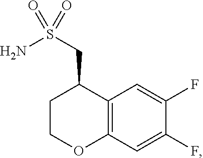 [SMILES: NS(=O)(=O)C[C@@H]1CCOc2cc(F)c(F)cc21]

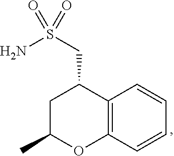 [SMILES: C[C@H]1C[C@H](CS(N)(=O)=O)c2ccccc2O1]

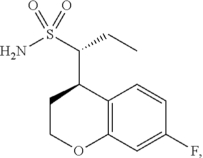 [SMILES: CC[C@H]([C@@H]1CCOc2cc(F)ccc21)S(N)(=O)=O]

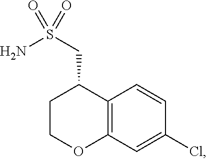 [SMILES: NS(=O)(=O)C[C@H]1CCOc2cc(Cl)ccc21]

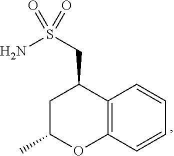 [SMILES: C[C@@H]1C[C@@H](CS(N)(=O)=O)c2ccccc2O1]

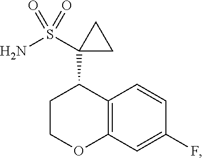 [SMILES: NS(=O)(=O)C1([C@H]2CCOc3cc(F)ccc32)CC1]

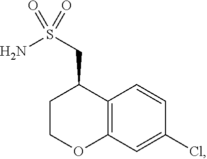 [SMILES: NS(=O)(=O)C[C@@H]1CCOc2cc(Cl)ccc21]

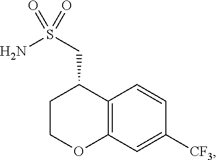 [SMILES: NS(=O)(=O)C[C@H]1CCOc2cc(C(F)(F)F)ccc21]

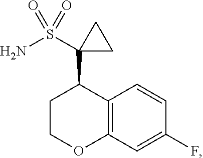 [SMILES: NS(=O)(=O)C1([C@@H]2CCOc3cc(F)ccc32)CC1]

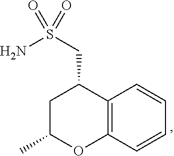 [SMILES: C[C@@H]1C[C@H](CS(N)(=O)=O)c2ccccc2O1]

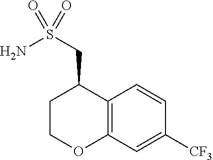 [SMILES: NS(=O)(=O)C[C@@H]1CCOc2cc(C(F)(F)F)ccc21]

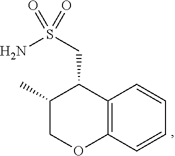 [SMILES: C[C@H]1COc2ccccc2[C@H]1CS(N)(=O)=O]

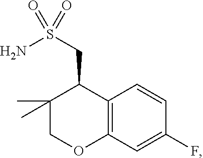 [SMILES: CC1(C)COc2cc(F)ccc2[C@@H]1CS(N)(=O)=O]

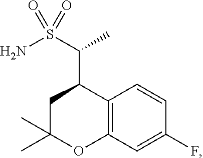 [SMILES: C[C@H]([C@@H]1CC(C)(C)Oc2cc(F)ccc21)S(N)(=O)=O]

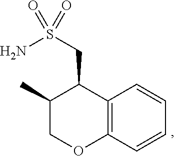 [SMILES: C[C@@H]1COc2ccccc2[C@@H]1CS(N)(=O)=O]

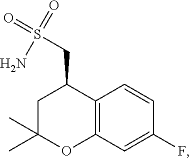 [SMILES: CC1(C)C[C@@H](CS(N)(=O)=O)c2ccc(F)cc2O1]

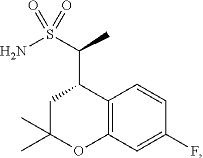 [SMILES: C[C@@H]([C@H]1CC(C)(C)Oc2cc(F)ccc21)S(N)(=O)=O]

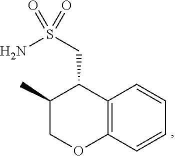 [SMILES: C[C@@H]1COc2ccccc2[C@H]1CS(N)(=O)=O]

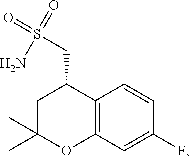 [SMILES: CC1(C)C[C@H](CS(N)(=O)=O)c2ccc(F)cc2O1]

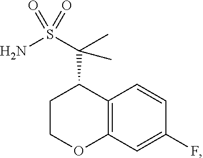 [SMILES: CC(C)([C@H]1CCOc2cc(F)ccc21)S(N)(=O)=O]

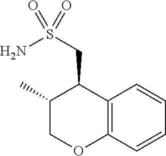 [SMILES: C[C@H]1COc2ccccc2[C@@H]1CS(N)(=O)=O]

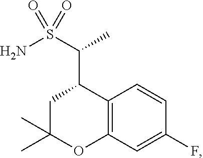 [SMILES: C[C@H]([C@H]1CC(C)(C)Oc2cc(F)ccc21)S(N)(=O)=O]

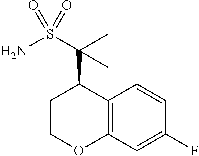 [SMILES: CC(C)([C@@H]1CCOc2cc(F)ccc21)S(N)(=O)=O]

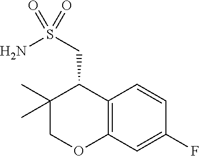 [SMILES: CC1(C)COc2cc(F)ccc2[C@H]1CS(N)(=O)=O]

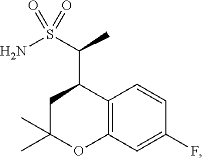 [SMILES: C[C@@H]([C@@H]1CC(C)(C)Oc2cc(F)ccc21)S(N)(=O)=O]

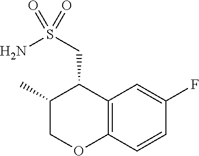 [SMILES: C[C@H]1COc2ccc(F)cc2[C@H]1CS(N)(=O)=O]

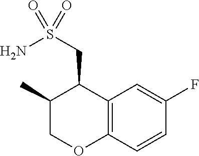 [SMILES: C[C@@H]1COc2ccc(F)cc2[C@@H]1CS(N)(=O)=O]

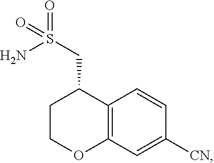 [SMILES: N#Cc1ccc2c(c1)OCC[C@@H]2CS(N)(=O)=O]

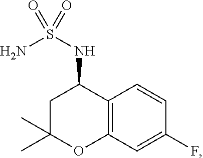 [SMILES: CC1(C)C[C@@H](NS(N)(=O)=O)c2ccc(F)cc2O1]

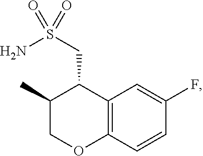 [SMILES: C[C@@H]1COc2ccc(F)cc2[C@H]1CS(N)(=O)=O]

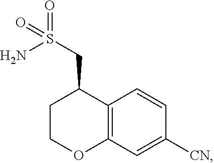 [SMILES: N#Cc1ccc2c(c1)OCC[C@H]2CS(N)(=O)=O]

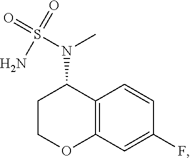 [SMILES: CN([C@H]1CCOc2cc(F)ccc21)S(N)(=O)=O]

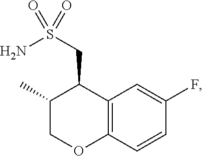 [SMILES: C[C@H]1COc2ccc(F)cc2[C@@H]1CS(N)(=O)=O]

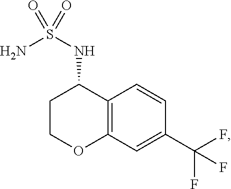 [SMILES: NS(=O)(=O)N[C@H]1CCOc2cc(C(F)(F)F)ccc21]

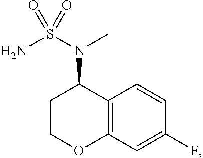 [SMILES: CN([C@@H]1CCOc2cc(F)ccc21)S(N)(=O)=O]

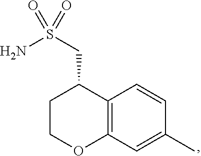 [SMILES: Cc1ccc2c(c1)OCC[C@@H]2CS(N)(=O)=O]

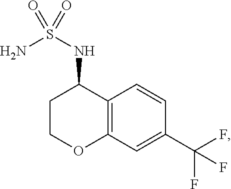 [SMILES: NS(=O)(=O)N[C@@H]1CCOc2cc(C(F)(F)F)ccc21]

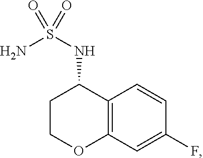 [SMILES: NS(=O)(=O)N[C@H]1CCOc2cc(F)ccc21]

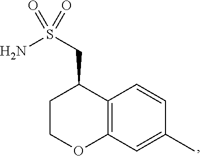 [SMILES: Cc1ccc2c(c1)OCC[C@H]2CS(N)(=O)=O]

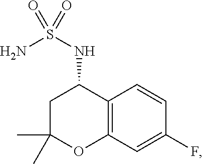 [SMILES: CC1(C)C[C@H](NS(N)(=O)=O)c2ccc(F)cc2O1]

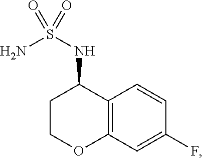 [SMILES: NS(=O)(=O)N[C@@H]1CCOc2cc(F)ccc21]

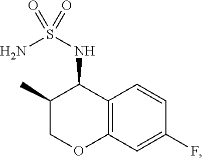 [SMILES: C[C@@H]1COc2cc(F)ccc2[C@@H]1NS(N)(=O)=O]

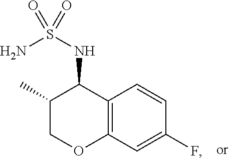 [SMILES: C[C@H]1COc2cc(F)ccc2[C@@H]1NS(N)(=O)=O ; "or"]

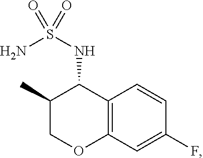 [SMILES: C[C@@H]1COc2cc(F)ccc2[C@H]1NS(N)(=O)=O]

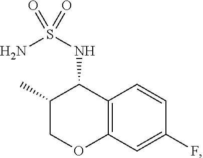 [SMILES: C[C@H]1COc2cc(F)ccc2[C@H]1NS(N)(=O)=O]

or a pharmaceutically acceptable salt of any of the aforementioned.

Methods

The Diagnostic and Statistical Manual of Mental Disorders, Fifth Ed., (the “DSM-5”), published by the American Psychiatric Association in 2013, and as amended or supplemented, provides a standard diagnostic system upon which persons of skill rely for diagnosis of various diseases and disorders, and is hereby incorporated by reference in its entirety. The DSM-5 attempts to capture the large proportion of patients with subsyndromal mixed symptoms with the inclusion of the mixed specifier. Additionally, the International Statistical Classification of Diseases (ICD 10) coding system is a recognized system to communicate about specific diagnoses (e.g., in the United States for billing purposes), and is hereby incorporated by reference in its entirety. For example, Chapter 6 of the ICD 10 is directed to codes for diseases of the nervous system.

The methods of the disclosure relate to the use of compounds and compositions disclosed herein to treat neurological or psychiatric diseases or disorders. In some embodiments, the neurological or psychiatric diseases or disorders is described in the DSM-5, as amended or supplemented, or the International Statistical Classification of Diseases (ICD 10) coding system.

Non-limiting examples of classes of neurological or psychiatric diseases or disorders include Movement Disorders, Cognitive Disorders, Pain, Neurodevelopmental Disorders; Schizophrenia Spectrum and Other Psychotic Disorders; Bipolar and Related Disorders; Depressive Disorders; Anxiety Disorders; Obsessive-Compulsive and Related Disorders; Trauma- and Stressor-Related Disorders; Dissociative Disorders; Somatic Symptom and Related Disorders; Feeding and Eating Disorders; Elimination Disorders; Sleep-Wake Disorders; Sexual Dysfunctions; Gender Dysphoria; Disruptive, Impulse-Control, and Conduct Disorders; Substance-Related and Addictive Disorders; Neurocognitive Disorders; Personality Disorders; Paraphilic Disorders; Other Mental Disorders; and Medication-Induced Movement Disorders and Other Adverse Effects of Medication.

Non-limiting examples of classes of neurological or psychiatric diseases or disorders include:

Movement Disorders

Tremor; Dyskinesia; Dystonia; Tics; Dysphonia; Ataxia (e.g., spinocerebellar ataxia); Myoclonus; Essential Tremor; Epilepsy; Tardive Dyskinesia; Restless Leg Syndrome; Tourette Syndrome; Multiple System Atrophy (MSA); Multiple Sclerosis; Huntington's Disease; Parkinson's Disease; Parkinsonism; Parkinson's disease tremor, Atypical Parkinsonisms (including, for example, Dementia with Lewy Bodies, Progressive Supranuclear Palsy, MSA and Corticobasal Syndrome); Wilson's Disease; damage or side effect from Stroke. Examples of akinesias and akinetic-rigid syndromes include Parkinson's disease, drug-induced Parkinsonism, postencephalitic Parkinsonism, secondary Parkinsonism, Parkinson plus syndromes, atypical Parkinsonism, idiopathic Parkinsonism, progressive supranuclear palsy, multiple system atrophy, corticobasal degeneration, Parkinsonism-ALS dementia complex and basal ganglia calcification, medication-induced Parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Gilles de la Tourette's syndrome, epilepsy, muscular spasms and disorders associated with muscular spasticity or weakness including tremors. Examples of dyskinesias include drug (e.g., L-DOPA) induced dyskinesia tremor (such as rest tremor, postural tremor, intention tremor), chorea (such as Sydenham's chorea, Huntington's disease, benign hereditary chorea, neuroacanthocytosis, symptomatic chorea, drug-induced chorea and hemiballism), myoclonus (including generalized myoclonus and focal myoclonus), tics (including simple tics, complex tics and symptomatic tics). Examples of dystonias include generalized dystonia, idiopathic dystonia, drug-induced dystonia, symptomatic dystonia, paroxysmal dystonia, focal dystonia, blepharospasm, oromandibular dystonia, spasmodic dysphonia, spasmodic torticollis, axial dystonia, dystonic writer's cramp and hemiplegic dystonia. Other examples of movement diseases or disorders include stereotypic movement disorder, persistent (chronic) motor disorder, medication-induced movement disorder, psychogenic movement disorders, substance/medication-induced movement disorder, extrapyramidal movement disorders, hyperkinetic movement disorders, hypokinetic movement disorders, alternating hemiplegia, Angelman syndrome, Hallervorden-Spatz Disease, ataxia, dentate cerebellar ataxia, ataxia telangiectasia (Louis-Bar syndrome), Friedreich's Ataxia, hereditary spinal ataxia, hereditary spinal sclerosis, Machado-Joseph Disease, spinocerebellar ataxia, progressive myoclonic ataxia, athetosis, ballismus, blepharospasm (eye twitching), cerebral palsy, tardive dystonia, tardive dyskinesia, idiopathic torsion dystonia, torsion dystonia, focal dystonia, idiopathic familial dystonia, idiopathic nonfamilial dystonia, cervical dystonia (spasmodic torticollis), primary dystonia, orofacial dystonia, developmental coordination disorder, bulbospinal muscular atrophy (Kennedy's Disease), Shy-Drager Syndrome, and Stiff-Person (Stiff-Man) Syndrome. In some embodiments, the present disclosure provides a method of treating one or more symptoms of epilepsy and/or seizures, including abdominal epilepsy, absence seizure, acquired epilepsy, acquired epileptiform aphasia, Aicardi syndrome, Alpers' disease, Alpers-Huttenlocher syndrome, Angelman syndrome, benign focal epilepsy, benign focal epilepsy of childhood, benign intracranial hypertension, benign rolandic epilepsy (BRE), CDKL5 disorder, childhood absence epilepsy, dentate cerebellar ataxia, Doose syndrome, Dravet syndrome, dyscognitive focal seizure, epilepsy with grand mal seizures, epilepsy with myoclonic-absences, epileptic hemiplegia, febrile seizures, focal seizure, frontal lobe epilepsy, generalized tonic-clonic seizures, genetic epilepsy, Glut1 deficiency syndrome, hypothalamic hamartoma, idiopathic epilepsy, idiopathic generalized epilepsy, idiopathic localization-related epilepsies, idiopathic partial epilepsy, idiopathic seizure, juvenile absence epilepsy, juvenile myoclonic epilepsy, Lafora disease, Lafora progressive myoclonus epilepsy, Landau-Kleffner syndrome, Lassueur-Graham-Little syndrome, Lennox syndrome, Lennox-Gastaut syndrome, medically refractory epilepsy, mesial-temporal lobe sclerosis, myoclonic seizure, neonatal epilepsy, occipital lobe epilepsy, Ohtahara syndrome, Panayiotopoulos syndrome, parietal lobe epilepsy, PCDH19 epilepsy, photosensitive epilepsy, progressive myoclonic epilepsies, Rasmussen's encephalitis, Rasmussen's syndrome, refractory epilepsy, seizure disorder, status epilepticus, Sturge-Weber syndrome, symptomatic generalized epilepsy, symptomatic partial epilepsy, TBCK-related ID syndrome, temporal lobe epilepsy, temporal lobe seizures, tonic-clonic seizure, West syndrome, tremor, cerebellar tremor, cerebellar outflow tremor, intention tremor, essential tremor, benign essential tremor, Parkinsonian tremor, and medication-induced postural tremor.

Cognitive Disorders

Alzheimer's disease; Cognitive Impairments; Dementia (including, e.g., Semantic Dementia; Frontotemporal Dementia; Dementia with Depressive Features; Persisting, Subcortical Dementia; Dementia with Lewy Bodies; Parkinsonism-ALS Dementia Complex; Dementia Associated with another disease or disorder, including Alzheimer's Disease; Ischemia; Multi-Infarct Dementia; Trauma; Vascular Problems; damage or side effect from Stroke; HIV Disease; Parkinson's Disease; Huntington's Disease; Down Syndrome; Pick's Disease; Creutzfeldt-Jacob Disease; Perinatal Hypoxia, or Substance abuse), Delirium; Amnestic Disorders; or Age Related Cognitive Decline. Cognitive Disorders includes a decline in cognitive functions or cognitive domains, e.g., working memory, attention and vigilance, verbal learning and memory, visual learning and memory, reasoning and problem solving (e.g., executive function, speed of processing and/or social cognition). In particular, cognitive impairment may indicate deficits in attention, disorganized thinking, slow thinking, difficulty in understanding, poor concentration, impairment of problem solving, poor memory, difficulties in expressing thoughts, and/or difficulties in integrating thoughts, feelings and behavior, or difficulties in extinction of irrelevant thoughts. Cognitive Disorders can manifest as a deficit in cognition (cognitive domains as defined by the DSM-5 are: complex attention, executive function, learning and memory, language, perceptual-motor, social cognition); and is sometimes associated with a deficit in dopamine signaling; and is sometimes associated with basal ganglia dysfunction; and is sometimes associated with dysregulated locomotor activity; and is sometimes associated with impairment of prefrontal cortex functioning.

Pain

Fibromyalgia; Neuropathic Pain (including, e.g., post herpetic (or post-shingles) neuralgia, reflex sympathetic dystrophy/causalgia or nerve trauma, phantom limb pain, carpal tunnel syndrome, and peripheral neuropathy (such as diabetic neuropathy or neuropathy arising from chronic alcohol use)), Sensitization Accompanying Neuropathic Pain, Inflammatory Pain; Acute Pain; Nociceptive Pain; Arthritis Pain; Rheumatoid Arthritis; Osteoarthritis; Joint Pain; Musculoskeletal Pain; Back Pain; Dorsalgia; Bulging Disc; Hip Pain; Visceral Pain; Headache; Tension Headache; Acute Tension Headache; Chronic Tension Headache; Chronic Cluster Headache; Common Migraine; Classic Migraine; Cluster Headache; Mixed Headache; Post-Traumatic Headache; Eye Strain Headache; Short-Lasting Unilateral Neuralgiform (SUNCT) Headache; SUNCT Syndrome, Herpes Zoster; Acute Herpes Zoster; Shingles; Postherpetic Neuralgia (Shingles); Causalgia; Central Pain; Central Pain Syndrome; Chronic Back Pain; Neuralgia; Neuropathic Pain Syndrome; Neuropathy; Diabetic Neuropathy; Diabetes-Related Neuropathy; Diabetes-Related Nerve Pain; Fibrositis; Peripheral Neuropathy Caused by Chemotherapy; Peripheral Nerve Disease; Peripheral Neuropathy; Nerve Pain; Nerve Trauma; Sensitization Accompanying Neuropathic Pain; Complex Regional Pain Syndrome; Compression Neuropathy; Craniofacial Pain; Chronic Joint Pain; Chronic Knee Pain; Chronic Pain Syndrome; Cancer Pain; Trigeminal Neuralgia; Tic Doloreaux; Reflex Sympathetic Causalgia; Painful Peripheral Neuropathy; Spinal Nerve Injury; Arachnoiditis; Spinal Pain; Bernhardt-Roth Syndrome (Meralgia Parasthetica); Carpal Tunnel Syndrome; Cerebrospinal Fluid Syndrome; Charcot-Marie-Tooth Disease; Hereditary Motor and Sensory Neuropathy; Peroneal Muscular Atrophy; Cluster-Tic Syndrome; Coccygeal Pain Syndromes; Compartment Syndrome; Degenerative Disc Disease; Failed Back Surgery Syndrome; Genito-Pelvic Pain/Penetration Disorder; Gout; Inflammatory Pain; Lumbar Radiculopathy; Neuroma (Painful Scar); Pain Associated with Multiple Sclerosis; Pelvic Floor Disorders; Phantom Limb Pain; Piriformis Syndrome; Psychogenic Pain; Radicular Pain Syndrome; Raeder's Syndrome; Referred Pain; Reflex Sympathetic Dystrophy Syndrome; Sciatica; Sciatica Pain: Scoliosis; Slipped Disc; Somatic Pain; Spinal Stenosis; Stiff-Person Syndrome/Stiff-Man Syndrome; Stump Pain; Sympathetically Maintained Pain; Tolosa-Hunt Syndrome; Whiplash; Pain Associated with Lyme Disease.

Neurodevelopmental Disorders

Intellectual Disability (Intellectual Developmental Disorder); Global Developmental Delay; Unspecified Intellectual Disability (Intellectual Developmental Disorder); Language Disorder; Speech Sound Disorder; Childhood-Onset Fluency Disorder (Stuttering); Social (Pragmatic) Communication Disorder; Unspecified Communication Disorder; Autism Spectrum Disorder (including, e.g., Asperger's syndrome; Pervasive Developmental Disorder; Rett Syndrome; and Fragile X Syndrome); Attention-Deficit/Hyperactivity Disorder; Other Specified Attention-Deficit/Hyperactivity Disorder; Unspecified Attention-Deficit/Hyperactivity Disorder; Specific Learning Disorder; Childhood Learning Disorder; Developmental Coordination Disorder; Stereotypic Movement Disorder; Tic Disorders; Other Specified Tic Disorder; Unspecified Tic Disorder; Other Specified Neurodevelopmental Disorder; Unspecified Neurodevelopmental Disorder.

Schizophrenia Spectrum and Other Psychotic Disorders

Schizotypal (Personality) Disorder; Delusional Disorder; Brief Psychotic Disorder; Shared Psychotic Disorder Schizophreniform Disorder; Schizophrenia (paranoid, disorganized, catatonic, or undifferentiated); Schizoaffective Disorder; Substance/Medication-Induced Psychotic Disorder; Psychotic Disorder Due to Another Medical Condition; Catatonia Associated With Another Mental Disorder (Catatonia Specifier); Catatonic Disorder Due to Another Medical Condition; Unspecified Catatonia; Other Specified Schizophrenia Spectrum and Other Psychotic Disorder; Unspecified Schizophrenia Spectrum and Other Psychotic Disorder. Schizophrenia is a disorder of unknown origin, which usually appears for the first time in early adulthood and is marked by characteristics such as psychotic symptoms, phasic progression and development, and/or deterioration in social behavior and professional capability. Characteristic psychotic symptoms are disorders of thought content (e.g., multiple, fragmentary, incoherent, implausible or simply delusional contents, or ideas of persecution) and of mentality (e.g., loss of association, flight of imagination, incoherence up to incomprehensibility), as well as disorders of perceptibility (e.g., hallucinations), emotions (e.g., superficial or inadequate emotions), self-perceptions, intentions, impulses, and/or inter-human relationships, and psychomotoric disorders (e.g., catatonia). Other symptoms are also associated with this disorder. Schizophrenia is classified into subgroups: the paranoid type, characterized by delusions and hallucinations and absence of thought disorder, disorganized behavior, and affective flattening; the disorganized type, also named “hebephrenic schizophrenia,” in which thought disorder and flat affect are present together; the catatonic type, in which prominent psychomotor disturbances are evident, and symptoms may include catatonic stupor and waxy flexibility; and the undifferentiated type, in which psychotic symptoms are present but the criteria for paranoid, disorganized, or catatonic types have not been met. The symptoms of schizophrenia normally manifest themselves in three broad categories: positive, negative and cognitive symptoms. Positive symptoms are those which represent an “excess” of normal experiences, such as hallucinations and delusions. Negative symptoms are those where the subject suffers from a lack of normal experiences, such as anhedonia and lack of social interaction. The cognitive symptoms relate to cognitive impairment in schizophrenics, such as lack of sustained attention and deficits in decision making.

Bipolar and Related Disorders

Bipolar I Disorder; Bipolar II Disorder; Cyclothymic Disorder; Substance/Medication-Induced Bipolar and Related Disorder; Bipolar and Related Disorder Due to Another Medical Condition; Other Specified Bipolar and Related Disorder; Unspecified Bipolar and Related Disorder; Specifiers for Bipolar and Related Disorders. Bipolar disorders (including both bipolar I and bipolar II) are serious psychiatric disorders that have a prevalence of approximately 2% of the population, and affects both genders alike. It is a relapsing-remitting condition characterized by cycling between elevated (i.e., manic) and depressed moods, which distinguishes it from other disorders such as major depressive disorder and schizophrenia. Bipolar I is defined by the occurrence of a full manic episode, although most individuals experience significant depression. Symptoms of mania include elevated or irritable mood, hyperactivity, grandiosity, decreased need for sleep, racing thoughts and in some cases, psychosis. The depressive episodes are characterized by anhedonia, sad mood, hopelessness, poor self-esteem, diminished concentration and lethargy. Bipolar II is defined as the occurrence of a major depressive episode and hypomanic (less severe mania) episode although subjects spend considerably more time in the depressive state. Other related conditions include cyclothymic disorder.

Depressive Disorders

Depression, Disruptive Mood Dysregulation Disorder; Major Depressive Disorder (MDD) (Unipolar Depression); Persistent Depressive Disorder (Dysthymia); Premenstrual Dysphoric Disorder; Substance/Medication-Induced Depressive Disorder; Treatment-Resistant Depression; Depressive Disorder Due to Another Medical Condition; Other Specified Depressive Disorder; Unspecified Depressive Disorder

Anxiety Disorders

Anxiety; Separation Anxiety Disorder; Selective Mutism; Specific Phobia; Social Anxiety Disorder (Social Phobia); Panic Disorder; Panic Attack Specifier; Agoraphobia; Generalized Anxiety Disorder; Substance/Medication-Induced Anxiety Disorder; Anxiety Disorder Due to Another Medical Condition; Other Specified Anxiety Disorder; Unspecified Anxiety Disorder. Anxiety disorders are characterized by fear, worry, and uneasiness, usually generalized and unfocused as an overreaction to a situation. Anxiety disorders differ in the situations or types of objects that induce fear, anxiety, or avoidance behavior, and the associated cognitive ideation. Anxiety differs from fear in that anxiety is an emotional response to a perceived future threat while fear is associated with a perceived or real immediate threat. They also differ in the content of the associated thoughts or beliefs. Examples of anxiety disorders include separation anxiety disorder, selective mutism, specific phobia, social anxiety disorder (social phobia), panic disorder, panic attack specifier, agoraphobia, generalized anxiety disorder, substance/medication-induced anxiety disorder, anxiety disorder due to another medical condition, illness anxiety disorder, social (pragmatic) communication disorder, other specified anxiety disorder, and unspecified anxiety disorder; stressor-related disorders, including reactive attachment disorder, disinhibited social engagement disorder, posttraumatic stress disorder (PTSD), acute stress disorder, and adjustment disorders.

Obsessive-Compulsive and Related Disorders

Obsessive-Compulsive Disorder; Body Dysmorphic Disorder; Hoarding Disorder; Trichotillomania (Hair-Pulling Disorder); Excoriation (Skin-Picking) Disorder; Substance/Medication-Induced Obsessive-Compulsive and Related Disorder; Obsessive-Compulsive and Related Disorder Due to Another Medical Condition; Other Specified Obsessive-Compulsive and Related Disorder; Unspecified Obsessive-Compulsive and Related Disorder.

Trauma- and Stressor-Related Disorders

Reactive Attachment Disorder; Disinhibited Social Engagement Disorder; Posttraumatic Stress Disorder; Acute Stress Disorder; Adjustment Disorders; Other Specified Trauma- and Stressor-Related Disorder; Unspecified Trauma- and Stressor-Related Disorder.

Dissociative Disorders

Dissociative Identity Disorder; Dissociative Amnesia; Depersonalization/Derealization Disorder; Other Specified Dissociative Disorder; Unspecified Dissociative Disorder.

Somatic Symptom and Related Disorders

Somatic Symptom Disorder; Illness Anxiety Disorder; Conversion Disorder (Functional Neurological Symptom Disorder); Psychological Factors Affecting Other Medical Conditions; Factitious Disorder; Other Specified Somatic Symptom and Related Disorder; Unspecified Somatic Symptom and Related Disorder.

Feeding and Eating Disorders

Pica; Rumination Disorder; Avoidant/Restrictive Food Intake Disorder; Anorexia Nervosa; Bulimia Nervosa; Binge-Eating Disorder; Other Specified Feeding or Eating Disorder; Unspecified Feeding or Eating Disorder.

Elimination Disorders

Enuresis; Encopresis; Other Specified Elimination Disorder; Unspecified Elimination Disorder.

Sleep-Wake Disorders

Insomnia Disorder; Hypersomnolence Disorder; Narcolepsy; Obstructive Sleep Apnea Hypopnea; Central Sleep Apnea; Sleep-Related Hypoventilation; Circadian Rhythm Sleep-Wake Disorders; Non-Rapid Eye Movement Sleep Arousal Disorders; Nightmare Disorder; Rapid Eye Movement (REM) Sleep Behavior Disorder; Restless Legs Syndrome; Substance/Medication-Induced Sleep Disorder; Other Specified Insomnia Disorder; Unspecified Insomnia Disorder; Other Specified Hypersomnolence Disorder; Unspecified Hypersomnolence Disorder; Other Specified Sleep-Wake Disorder; Unspecified Sleep-Wake Disorder.

Sexual Dysfunctions

Delayed Ejaculation; Erectile Disorder; Female Orgasmic Disorder; Female Sexual Interest/Arousal Disorder; Genito-Pelvic Pain/Penetration Disorder; Male Hypoactive Sexual Desire Disorder; Premature (Early) Ejaculation; Substance/Medication-Induced Sexual Dysfunction; Other Specified Sexual Dysfunction; Unspecified Sexual Dysfunction.

Gender Dysphoria

Gender Dysphoria; Other Specified Gender Dysphoria; Unspecified Gender Dysphoria.

Disruptive, Impulse-Control, and Conduct Disorders

Social Disorder; Oppositional Defiant Disorder; Intermittent Explosive Disorder; Conduct Disorder; Antisocial Personality Disorder; Pyromania; Kleptomania; Other Specified Disruptive, Impulse-Control, and Conduct Disorder; Unspecified Disruptive; Impulse-Control, and Conduct Disorder.

Substance-Related and Addictive Disorders

Addiction; Alcohol Use Disorder; Alcohol Intoxication; Alcohol Withdrawal; Unspecified Alcohol-Related Disorder; Fetal Alcohol Syndrome; Caffeine Intoxication; Caffeine Withdrawal; Unspecified Caffeine-Related Disorder; Cannabis Use Disorder; Cannabis Intoxication; Cannabis Withdrawal; Unspecified Cannabis-Related Disorder; Phencyclidine Use Disorder; Other Hallucinogen Use Disorder; Phencyclidine Intoxication; Other Hallucinogen Intoxication; Hallucinogen Persisting Perception Disorder; Unspecified Phencyclidine-Related Disorder; Unspecified Hallucinogen-Related Disorder; Inhalant Use Disorder; Inhalant Intoxication; Unspecified Inhalant-Related Disorder; Opioid Use Disorder; Opioid Intoxication; Opioid Withdrawal; Unspecified Opioid-Related Disorder; Sedative, Hypnotic, or Anxiolytic Use Disorder; Sedative, Hypnotic, or Anxiolytic Intoxication; Sedative, Hypnotic, or Anxiolytic Withdrawal; Unspecified Sedative-, Hypnotic-, or Anxiolytic-Related Disorder; Stimulant Use Disorder; Stimulant Intoxication; Stimulant Withdrawal; Unspecified Stimulant-Related Disorder; Tobacco Use Disorder; Tobacco Withdrawal; Unspecified Tobacco-Related Disorder; Other (or Unknown) Substance Use Disorder; Other (or Unknown) Substance Intoxication; Other (or Unknown) Substance Withdrawal; Unspecified Other (or Unknown) Substance-Related Disorder; Gambling Disorder.

Neurocognitive Disorders

Delirium; Other Specified Delirium; Unspecified Delirium; Major and Mild Neurocognitive Disorders; Major or Mild Neurocognitive Disorder Due to Alzheimer's Disease; Major or Mild Frontotemporal Neurocognitive Disorder; Major or Mild Neurocognitive Disorder With Lewy Bodies; Major or Mild Vascular Neurocognitive Disorder; Major or Mild Neurocognitive Disorder Due to Traumatic Brain Injury; Substance/Medication-Induced Major or Mild Neurocognitive Disorder; Major or Mild Neurocognitive Disorder Due to HIV Infection; Major or Mild Neurocognitive Disorder Due to Prion Disease; Major or Mild Neurocognitive Disorder Due to Parkinson's Disease; Major or Mild Neurocognitive Disorder Due to Huntington's Disease; Major or Mild Neurocognitive Disorder Due to Another Medical Condition; Major or Mild Neurocognitive Disorder Due to Multiple Etiologies; Unspecified Neurocognitive Disorder.

Personality Disorders

Dimensional Models for Personality Disorders; General Personality Disorder; Paranoid Personality Disorder; Schizoid Personality Disorder; Schizotypal Personality Disorder; Antisocial Personality Disorder; Borderline Personality Disorder; Histrionic Personality Disorder; Narcissistic Personality Disorder; Avoidant Personality Disorder; Dependent Personality Disorder; Obsessive-Compulsive Personality Disorder; Personality Change Due to Another Medical Condition; Other Specified Personality Disorder; Unspecified Personality Disorder.

Paraphilic Disorders

Voyeuristic Disorder; Exhibitionistic Disorder; Frotteuristic Disorder; Sexual Masochism Disorder; Sexual Sadism Disorder; Pedophilic Disorder; Fetishistic Disorder; Transvestic Disorder; Other Specified Paraphilic Disorder; Unspecified Paraphilic Disorder.

Other Mental Disorders

Other Specified Mental Disorder Due to Another Medical Condition; Unspecified Mental Disorder Due to Another Medical Condition; Other Specified Mental Disorder; Unspecified Mental Disorder.

Medication-Induced Movement Disorders and Other Adverse Effects of Medication

Neuroleptic-Induced Parkinsonism Other Medication-Induced Parkinsonism; Neuroleptic Malignant Syndrome; Medication-Induced Acute Dystonia; Medication-Induced Acute Akathisia; Tardive Dyskinesia; Tardive Dystonia Tardive Akathisia; Medication-Induced Postural Tremor; Other Medication-Induced Movement Disorder; Antidepressant Discontinuation Syndrome; Other Adverse Effect of Medication.

Symptoms of Neurological or Psychiatric Diseases and Disorders

Neurological or psychiatric diseases or disorders can manifest as a variety of symptoms. Non-limiting examples of symptoms of neurological or psychiatric diseases or disorders include symptoms such as apathy, depression, anxiety, cognitive impairment, psychosis, aggression, agitation, impulse control disorders, sleep disorders, elevated or irritable mood, hyperactivity, grandiosity, decreased need for sleep, racing thoughts and in some cases, psychosis, anhedonia, sad mood, hopelessness, poor self-esteem, diminished concentration and lethargy, amyotrophic lateral sclerosis, primary lateral sclerosis, progressive muscular atrophy, progressive bulbar (atrophy) palsy, pseudobulbar palsy spinal muscular atrophy diseases (e.g., SMA type I, also called Werdnig-Hoffmann disease, SMA type IL, SMA type III, also called Kugelberg-Welander disease, and Kennedy Disease, also called progressive spinobulbar muscular atrophy), Hallervorden-Spatz disease, Seitelberger disease (Infantile Neuroaxonal Dystrophy), adrenoleukodystrophy, Alexander Disease, autosomal dominant cerebellar ataxia (ADCA), pure autonomic failure (Bradbury-Eggleston Syndrome), CADASIL Syndrome, and neuronal ceroids lipofuscinose disorders such as Batten Disease (Spielmeyer-Vogt-Sjögren)), senile dementia, Early Onset Alzheimer's Disease, Alzheimer's type dementia, cognition, memory loss, amnesia/amnestic syndrome, disturbances of consciousness, coma, lowering of attention, speech disorder, agnosia, aphasia, apraxia, Mild Cognitive Impairment (MCI), benign forgetfulness, mild neurocognitive disorder, major neurocognitive disorder, neurocognitive disorder due to disease (e.g., Huntington's Disease, Parkinson's disease, Prion Disease, Traumatic Brain Injury, HIV or AIDS), Binswanger's Disease (subcortical leukoencephalopathy), and Capgras Syndrome; or any other symptoms associated with a neurological or psychiatric disease or disorder disclosed herein.

Pharmaceutical Compositions

In certain embodiments, provided herein is a composition (e.g., a pharmaceutical composition) comprising a compound described herein and a pharmaceutically acceptable excipient or carrier. In some embodiments, provided herein is a method of treating neurological or psychiatric diseases and disorders in a subject in need thereof in a subject, comprising administering an effective amount of a compound or a pharmaceutical composition described herein. Examples of carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present disclosure or pharmaceutical composition thereof) or aid in the manufacturing of the pharmaceutical product (i.e., medicament).

Compositions of the present disclosure may be administered orally, parenterally, by inhalation, topically, rectally, nasally, buccally, sublingually, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. Pharmaceutically acceptable compositions of this disclosure may be orally administered in any orally acceptable dosage form including capsules, tablets, aqueous suspensions or solutions.

The amount of compounds of the present disclosure that may be combined with the carrier materials to produce a composition in a single dosage form will vary depending upon a variety of factors, including the host treated and the particular mode of administration. It should also be understood that a specific dosage and treatment regimen for any particular subject will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

In some embodiments, the compounds and compositions of the disclosure are formulated in dosage unit form for ease of administration and uniformity of dosage. The expression “dosage unit form” or “dosage form” as used herein refers to a physically discrete unit of agent appropriate for the subject to be treated. It will be understood, however, that the total daily usage of the compounds and compositions of the present disclosure will be decided by the attending physician within the scope of sound medical judgment.

Combination Therapy

In some embodiments, the present disclosure provides a method of treating a neurological and/or psychiatric disease or disorder described herein, comprising administering a compound of the disclosure in conjunction with one or more pharmaceutical agents. Suitable pharmaceutical agents that may be used in combination with the compounds of the present disclosure include essential tremor medications, Parkinson's disease medications, dystonia medications, cerebellar ataxia medications, Huntington's disease medications, multiple system atrophy medications.

Suitable essential tremor medications that can be used in combination with the compounds of the disclosure include, for example, beta blockers (e.g., propranolol, metoprolol), anticonvulsants (e.g., primidone, topiramate, zonisamide), benzodiazepines (e.g., lorazepam, diazepam, alprazolam, clonazepam), neurosteroids (e.g., GABA-A receptor positive allosteric modulators, allopregnanolone, zuranolone), SK channel positive allosteric modulators (e.g., CAD-1883), T-channel blockers (e.g., suvecaltamide). Further essential tremor medications include gabapentin and botulinum toxin. The compounds of the disclosure can also be combined with treatments for essential tremor that include, for example, deep brain simulation (DBS) and focused ultrasound thalamotomy.

Suitable Parkinson's disease medications that can be used in combination with the compounds of the disclosure include, for example, neurosteroids (e.g., GABA-A receptor positive allosteric modulators, allopregnanolone, zuranolone), T-channel blockers (e.g., suvecaltamide), levodopa, carbidopa, dopamine receptor agonists (e.g., pramipexole, ropinirole, apomorphine hydrochloride, rotigotine), MAO-B inhibitors (e.g., selegiline, rasagiline, safinamide, zonisamide), COMT inhibitors (e.g., entacopone, tolcapone, opicapone), adenosine A2a antagonists (e.g., istradefylline), anticholinergic drugs (benztropine, trihexyphenidyl, donepezil, rivastigmine, galantamine), 5-HT1A agonists (e.g., buspirone, sarizotan), mGluR55 antagonists (e.g., mavoglurant, dipraglurant), antipsychotics, 5-HT2A antagonists (e.g., clozapine, risperidone, olanzapine, quetiapine and ziprasidone, pimavanserin), and SEP-363856. Additional medications include, for example, amantadine. The compounds of the disclosure can also be combined with treatments for Parkinson's disease that include, for example, deep brain simulation (DBS).

Suitable dystonia medications that can be used in combination with the compounds of the disclosure include, for example, botulinum toxin, antipsychotics, 5-HT2A antagonists (e.g., clozapine, risperidone, olanzapine, quetiapine and ziprasidone, pimavanserin), anticholinergics (e.g., trihexyphenidyl), baclofen, and levodopa. The compounds of the disclosure can also be combined with treatments for dystonia that include, for example, deep brain simulation (DBS) and selective denervation surgery.

Suitable cerebellar ataxia medications that can be used in combination with the compounds of the disclosure include, for example, anticonvulsants (e.g., primidone, topiramate, zonisamide), SK channel positive allosteric modulators (e.g., CAD-1883), and riluzole. The compounds of the disclosure can also be combined with treatments for cerebellar ataxia treatments that include, for example, transcranial direct-current stimulation (TDCS) and transcranial magnetic stimulation (TMS).

Suitable multiple system atrophy medications include, for example, levodopa, carbidopa, pyridostigmine (cholinesterase inhibitor), fludrocortisone, midodrine (adrenergic al-receptor agonist), botulinum toxin, and midodrine (al adrenergic receptor agonist).

The compounds of the disclosure can also be used in combination with medications for the treatment of chorea and Huntington's disease (e.g., tetrabenazine and deutetrabenazine) and REM sleep behavioral disorder (e.g., melatonin, clonazepam).

Suitable pharmaceutical agents that may be used in combination with the compounds of the present disclosure include anti-Parkinson's drugs, anti-Alzheimer's drugs, anti-depressants, anti-psychotics, anti-ischemics, CNS depressants, anti-cholinergics, nootropics, epilepsy medication, attention (e.g., ADD/ADHD) medications, sleep-promoting medications, wakefulness-promoting medications, and pain medications. In some embodiments, suitable pharmaceutical agents are anxiolytics.

In some embodiments, compounds of the disclosure can be used in combination with levodopa (with or without a selective extracerebral decarboxylase inhibitor such as carbidopa or benserazide), anticholinergics such as biperiden (optionally as its hydrochloride or lactate salt) and trihexyphenidyl(benzhexyl)hydrochloride, COMT inhibitors such as entacapone or tolcapone, MAO A/B inhibitors, antioxidants, A2a adenosine receptor antagonists, cholinergic agonists, NMDA receptor antagonists, serotonin receptor antagonists and dopamine receptor agonists such as alentemol, bromocriptine, fenoldopam, lisuride, naxagolide, pergolide and pramipexole. It will be appreciated that the dopamine agonist may be in the form of a pharmaceutically acceptable salt, for example, alentemol hydrobromide, bromocriptine mesylate, fenoldopam mesylate, naxagolide hydrochloride and pergolide mesylate. Lisuride and pramipexole are commonly used in a non-salt form.

Suitable anti-Alzheimer's drugs include beta-secretase inhibitors, gamma-secretase inhibitors, cholinesterase inhibitors such as donepezil, galantamine or rivastigmine, HMG-CoA reductase inhibitors, NSAID's including ibuprofen, vitamin E, and anti-amyloid antibodies. In some embodiments, an anti-Alzheimer's drug is memantine.

Suitable anti-depressants and anti-anxiety agents include norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, a-adrenoreceptor antagonists, neurokinin-1 receptor antagonists, atypical anti-depressants, benzodiazepines, 5-HT1A agonists or antagonists, especially 5-HT1A partial agonists, and corticotropin releasing factor (CRF) antagonists.

Specific suitable anti-depressant and anti-anxiety agents include amitriptyline, clomipramine, doxepin, imipramine and trimipramine; amoxapine, desipramine, citalopram, escitalopram, maprotiline, nortriptyline and protriptyline; fluoxetine, fluvoxamine, paroxetine and sertraline; isocarboxazid, phenelzine, tranylcypromine and selegiline; moclobemide: venlafaxine; desvenlafaxine, duloxetine; aprepitant; bupropion, vilazodone, mirtazapine, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, reboxetine, vortioxetine, clorazepate, and ketamine and pharmaceutically acceptable salts thereof. In some embodiments, suitable anti-depressant and anti-anxiety agents are tianeptine, or pharmaceutically acceptable salts thereof.

Suitable anti-psychotic and mood stabilizer agents include D2 antagonists, 5HT2A antagonists, atypical antipsychotics, lithium, and anticonvulsants.

Specific suitable anti-psychotic and mood stabilizer agents include chlorpromazine, fluphenazine, haloperidol, amisulpride, perphenazine, thioridazine, trifluoperazine, aripiprazole, asenapine, clozapine, olanzapine, paliperidone, brexpiprazole, paliperidone, cariprazine, pimavanserin, illoperidone, lumateperone, MIN-101, quetiapine, risperidone, ziprasidone, lurasidone, flupentixol, levomepromazine, pericyazine, perphenazine, pimozide, prochlorperazine, zuclopenthixol, olanzapine and fluoxetine, lithium, carbamazepine, lamotrigine, valproic acid, iloperidone, thiothixene, gabapentin, tiagabine and pharmaceutically acceptable salts thereof.

Suitable epilepsy medications include levetiracetam, oxcarbazepine, clobazam, retigabine, zonisamide, felbamate, esclicarbazepine acetate, lacosamide, carbamazepine, tiagabine, methsuximide, progabide, valproic acid, lamotrigine, brivaracetam, rufinamide, topiramate and perampanel.

Suitable attention medications include methyl phenidate, atomoxetine, guanfacine, D-amphetamine, lisdexamphetamine, methylamphetamine, and clonidine.

Suitable sleep-promoting medications include ramelteon, triazolam, zopiclone, eszopiclone, zolpidem, temazepam, and trazodone.

Suitable wakefulness-promoting medications include Modafinil, D-Amphetamine, caffeine, and armodafinil.

Suitable pain medications include dextromethorphan, tapentadol, buprenorphine, codeine, fentanyl, hydrocodone, hydromorphone, morphine, naloxegol, oxycodone, tramadol, gabapentil, difluprednate, pregabalin, acetyl salicyclic acid, bromfenac, diclofenac, diflunisal, indomethacin, ketorolac, meoxican, and naproxen.

In some embodiments, compounds and compositions of the disclosure may be used in combination with other therapies. Suitable therapies include psychotherapy, cognitive behavioral therapy, electroconvulsive therapy, transcranial magnetic stimulation, vagus nerve stimulation, and deep-brain stimulation.

Other examples of agents the compounds and compositions of this disclosure may also be combined with include: vitamins and nutritional supplements, antiemetics (e.g., 5-HT3 receptor antagonists, dopamine antagonists, NK1 receptor antagonists, histamine receptor antagonists, cannabinoids, benzodiazepines, or anticholinergics), agents for treating Multiple Sclerosis (MS) such as beta interferon (e.g., Avonex® and Rebif®, dalfampridine, alemtuzumab), Copaxone®, and mitoxantrone; treatments for Huntington's disease such as tetrabenazine; treatments for asthma such as albuterol and Singulair®; anti-inflammatory agents such as corticosteroids, TNF blockers, IL-1 RA, azathioprine, and sulfasalazine; immunomodulatory and immunosuppressive agents such as cyclosporin, tacrolimus, rapamycin, mycophenolate mofetil, interferons, corticosteroids, cyclophosphamide, azathioprine, and sulfasalazine; neurotrophic factors such as acetylcholinesterase inhibitors, MAO inhibitors, interferons, anti-convulsants, ion channel blockers, riluzole, agents for treating cardiovascular disease such as beta-blockers, ACE inhibitors, diuretics, nitrates, calcium channel blockers, and statins, fibrates, cholesterol absorption inhibitors, bile acid sequestrants, and niacin; agents for treating liver disease such as corticosteroids, cholestyramine, interferons, and anti-viral agents; agents for treating blood disorders such as corticosteroids, anti-leukemic agents, and growth factors; agents for treating immunodeficiency disorders such as gamma globulin; and anti-diabetic agents such as biguanides (metformin, phenformin, buformin), thiazolidinediones (rosiglitazone, pioglitazone, troglitazone), sulfonylureas (tolbutamide, acetohexamide, tolazamide, chlorpropamide, glipizide, glyburide, glimepiride, gliclazide), meglitinides (repaglinide, nateglinide), alpha-glucosidase inhibitors (miglitol, acarbose), incretin mimetics (exenatide, liraglutide, taspoglutide), gastric inhibitory peptide analogs, DPP-4 inhibitors (vildagliptin, sitagliptin, saxagliptin, linagliptin, alogliptin), amylin analogs (pramlintide), and insulin and insulin analogs.

In some embodiments, a compound of the present disclosure, or a pharmaceutically acceptable salt thereof, is administered in combination with an antisense agent, a monoclonal or polyclonal antibody, or an siRNA therapeutic.

In some embodiments, one or more additional agents may be combined with a compound of formula I disclosed herein, or a pharmaceutically acceptable salt thereof. As used herein, the term “combination,” “combined,” and related terms refers to the simultaneous, contemporaneous, or sequential administration of one or more therapeutic agents in addition to a compound of formula I disclosed herein, or a pharmaceutically acceptable salt thereof. In some embodiments, the one or more additional agents may be administered separately from a composition including a compound of formula I disclosed herein, or a pharmaceutically acceptable salt thereof, as part of a multiple dosage regimen. Alternatively, in some embodiments, the one or more additional agents may be part of a single dosage form including the one or more additional agents combined together with a compound of formula I disclosed herein, or a pharmaceutically acceptable salt thereof, in a single composition. In some embodiments, the two or more active agents (i.e., a compound of formula I disclosed herein, or a pharmaceutically acceptable salt thereof, and one or more additional agents) may be administered simultaneously as part of a multiple dosage regime. Alternatively, in some embodiments, the two or more active agents may be administered sequentially within a discrete period of time spacing the dosage intervals or on a schedule of administration over a longer period of time as part of a multiple dosage regime. In some embodiments, a sequential administration of one or more additional agents occurs within five hours of the initial administration of a compound of formula I disclosed herein, or a pharmaceutically acceptable salt thereof. In some embodiments, a sequential administration of a compound of formula I disclosed herein, or a pharmaceutically acceptable salt thereof, occurs within five hours of the initial administration of one or more additional agents.

In some embodiments, the amount of a compound of formula I disclosed herein, or a pharmaceutically acceptable salt thereof and one or more additional agents (in those compositions which comprise an additional therapeutic agent as described above) that may be combined with the carrier materials to produce a single dosage form will vary depending upon the host treated and the particular mode of administration. In some embodiments, the composition may be formulated so that a dosage of between 0.01-100 mg/kg body weight/day of compound of formula I disclosed herein, or a pharmaceutically acceptable salt thereof can be administered.

In those compositions which comprise an additional therapeutic agent, that additional therapeutic agent and the compound of this disclosure may act synergistically. Therefore, the amount of additional therapeutic agent in such compositions will be less than that required in a monotherapy utilizing only that therapeutic agent. In such compositions a dosage of between 0.01-100 mg/kg body weight/day of the additional therapeutic agent can be administered.

The amount of additional therapeutic agent present in the compositions of this disclosure will be no more than the amount that would normally be administered in a composition comprising that therapeutic agent as the only active agent. In some embodiments, the amount of additional therapeutic agent in the presently disclosed compositions will range from about 50% to 100% of the amount normally present in a composition comprising that agent as the only therapeutically active agent.

### EXAMPLES

The compounds of the present disclosure can be prepared in a number of ways known to one skilled in the art of organic synthesis in view of the methods, reaction schemes and examples provided herein. The compounds of the present disclosure can be synthesized using the methods described below, together with synthetic methods known in the art of synthetic organic chemistry, or by variations thereon, as appreciated by those skilled in the art. Preferred methods include, but are not limited to, those described below. The reactions are performed in a solvent or solvent mixture appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the disclosure. Examples are depicted with relative stereochemistry except where specifically stated otherwise.

The starting materials are generally available from commercial sources such as Sigma Aldrich or other commercial vendors, or are prepared as described in this disclosure, or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), Larock, R. C., Comprehensive Organic Transformations, 2^{nd}ed., Wiley-VCH Weinheim, Germany (1999), or Beilsteins Handbuch der organischen Chemie, 4, Aufl. ed. Springer-Verlag, Berlin, including supplements (also available via the Beilstein online database).

For illustrative purposes, the reaction schemes depicted below provide potential routes for synthesizing the compounds of the present disclosure as well as key intermediates. Those skilled in the art will appreciate that other synthetic routes may be used to synthesize the compounds of the present disclosure. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

In the preparation of compounds of the present disclosure, protection of remote functionality of intermediates may be necessary. The need for such protection will vary depending on the nature of the remote functionality and the conditions of the preparation methods. The need for such protection is readily determined by one skilled in the art. For a general description of protecting groups and their use, see Greene, T. W. et al., Protecting Groups in Organic Synthesis, 4th Ed., Wiley (2007). Protecting groups incorporated in making of the compounds of the present disclosure, such as the trityl protecting group, may be shown as one regioisomer but may also exist as a mixture of regioisomers.

The following abbreviations used herein have the corresponding meanings:

| | | | |
|---|---|---|---|
| ACN | acetonitrile | aq. | Aqueous |
| t-BuLi | tert-butyllithium | | |
| BINAP | 2,2′-Bis(diphenylphosphino)-1,l'-binaphthyl | | |
| Boc | tert-butyloxycarbonyl | C | Celsius |
| DCM | dichloromethane | d | doublet |
| dd | doublet of doublets | DMF | N,N-dimethylformamide |
| DIPEA | N,N-diisopropylethylamine | | |
| DMF•DMA | N,N-dimethylformamide dimethyl acetal | | |
| DMSO | dimethylsulfoxide | EtOAc | ethyl acetate |
| EtOH | ethanol | g | gram |
| h | hour(s) | HCl | hydrochloric acid |
| HPLC | high pressure liquid chromatography | | |
| kg | kilogram | L | liter |
| LCMS | liquid chromatography and mass spectrometry | | |
| MeOH | methanol | MS | mass spectrometry |
| M | molar | M | multiplet |
| Min | minutes | mL | milliliter(s) |
| m/z | mass to charge ratio | NaH | sodium hydride |
| NaOH | sodium hydroxide | NaCl | sodium chloride |
| Na₂SO₄ | sodium sulphate | N | normal |
| NH₄Cl | ammonium chloride | NMP | N-methylpyrrolidone |
| NMR | nuclear magnetic resonance | | |
| Pd(OAc)₂ | palladium(II) acetate | PE | petroleum ether |
| K₂CO₃ | potassium carbonate | PCC | pyridinium chlorochromate |
| PG | protecting group | pTsOH | p-toluenesulfonic acid |
| rac | racemic | rt | room temperature |
| s | singlet | sat. | saturated |
| t | triplet | TEA | triethylamine |
| TES | triethylsilane | TFA | trifluoroacetic acid |
| THF | tetrahydrofuran | TLC | thin layer chromatography |

### Example 1. Preparation of (R*)-(7-fluorospiro[chromane-2,1′-cyclopropan]-4-yl)methanesulfonamide (Compound 2) and (S*)-(7-fluorospiro[chromane-2,1′-cyclopropan]-4-yl)methanesulfonamide (Compound 1)

### Synthesis of ethyl 2-cyclopropylideneacetate

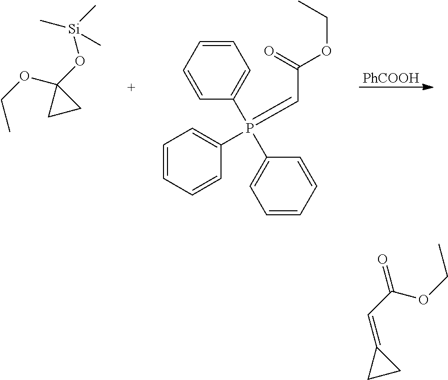

To a solution of (1-ethoxycyclopropoxy)trimethylsilane (5.0 g, 28.6 mmol) and ethyl 2-(triphenyl-phosphanylidene)acetate (12.9 g, 37.4 mmol) in toluene (150 mL) was added benzoic acid (0.84 g, 6.9 mmol) at about room temperature. The mixture was stirred at about 80° C. for about 10 hours and then concentrated. The resulting substance was purified by silica gel column (PE/EtOAc=50/1) to give ethyl 2-cyclopropylideneacetate.

### Synthesis of ethyl 2-(1-(3-fluorophenoxy)cyclopropyl)acetate

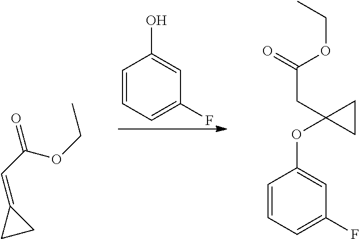

A mixture of ethyl 2-cyclopropylideneacetate (1.0 g, 16.8 mmol), 3-fluorophenol (2.65 g, 23.7 mmol) and 4 A molecular sieve (1.0 g) in DMF (20 mL) was stirred at about 130° C. for about 15 hours. Water (50 mL) was added to the mixture, and the mixture was then extracted with EtOAc (100 mL×2). The combined organic phase was dried and concentrated to give the crude product, which was purified by silica gel column chromatography (PE/EtOAc=10/1) to give the desired product. MS(ESI): m/z 239 [M+H]⁺.

### Synthesis of 2-(1-(3-fluorophenoxy)cyclopropyl)acetic Acid

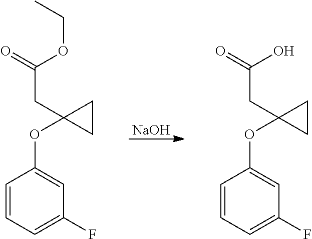

A solution of (ethyl 2-[1-(3-fluorophenoxy) cyclopropyl] acetate (1.4 g, 5.9 mmol), NaOH (0.71 g, 17.7 mmol) in MeOH/H₂O (25 mL, 4:1) was stirred at about room temperature for about 10 hours. After the pH value of the reaction mixture was adjusted to 7 with acetic acid, the reaction was concentrated. Water (50 mL) was added, and the mixture was extracted with ethyl acetate (100 mL×2). The combined organic phases were dried and concentrated in vacuo. The residue was purified by silica gel column (PE/EtOAc=5/1). MS (ESI): m/z 211 [M+H]⁺.

### Synthesis of 7-fluorospiro[chroman-2,1′-cyclopropan]-4-one

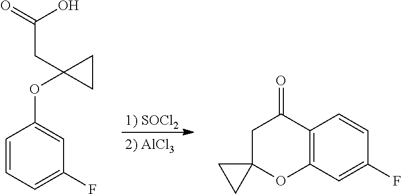

To a solution of 2-[1-(3-fluorophenoxy)cyclopropyl]acetic acid (1.0 g, 4.76 mmol) in DCM (20 mL) was added thionyl chloride (1.41 g, 11.9 mmol) at about room temperature. The mixture was stirred at about room temperature for about 10 hours and then concentrated in vacuo. The residue was dissolved in DCM (20 mL) and aluminum trichloride (0.75 g, 5.68 mmol) in DCM (10 mL) was added to the above solution slowly at about 0° C. After the mixture was stirred at this temperature for about 0.5 hours, water (30 mL) was added at 0° C. slowly. The mixture was stirred at this temperature for about another 0.5 hours, and then extracted with DCM (50 mL×2). The combined organic phases were dried and concentrated. The crude product was purified by silica gel column (PE/EtOAc=10/1) to give 7-fluorospiro[chroman-2,1′-cyclopropan]-4-one. MS(ESI): m/z 193 (M+H)+.

### Synthesis of tert-butyl (7-fluoro-4-hydroxyspiro[chroman-2,1′-cyclopropane]-4-yl)methylsulfonylcarbamate

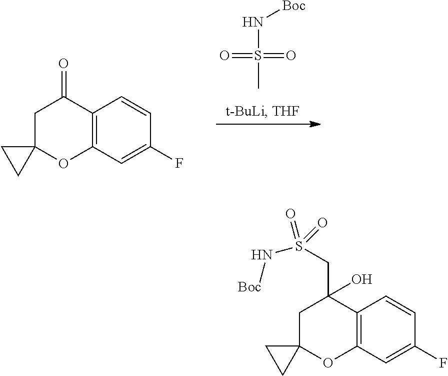 [SMILES: O=C1CC2(CC2)Oc2cc(F)ccc21 ; CC(C)(C)OC(=O)NS(C)(=O)=O ; CC(C)(C)OC(=O)NS(=O)(=O)CC1(O)CC2(CC2)Oc2cc(F)ccc21]

To a solution of tert-butyl methylsulfonylcarbamate (1.2 g, 6.1 mmol) in THF (30 mL) was added t-BuLi (1.3M in pentane, 9.4 mL, 12.1 mmol) dropwise at about −78° C., and the reaction was stirred at this temperature for about 1 hour. A solution of 7-fluorospiro[chroman-2,1′-cyclopropan]-4-one (0.9 g, 4.7 mmol) in THF (10 mL) was added and the mixture was stirred at this temperature for about 3 hours. The reaction was quenched with saturated ammonia chloride (aq. 20 mL) and extracted with EtOAc (50 mL×2). The combined organic phases were washed with brine, dried and concentrated in vacuo. The crude product was purified by chromatography on silica gel with PE/EtOAc=5/1. MS (ESI): m/z 405 [M+Na]⁺.

### Synthesis of (7-fluorospiro[chromane-2,1′-cyclopropan]-4-yl)methanesulfonamide

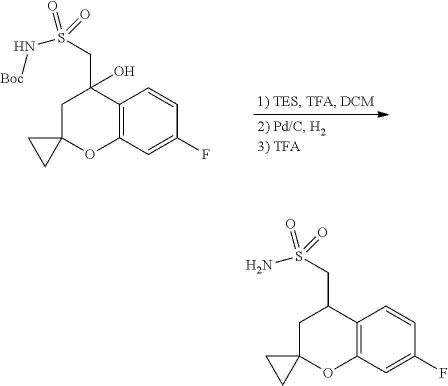 [SMILES: CC(C)(C)OC(=O)NS(=O)(=O)CC1(O)CC2(CC2)Oc2cc(F)ccc21 ; NS(=O)(=O)CC1CC2(CC2)Oc2cc(F)ccc21]

To a solution of tert-butyl N-({7-fluoro-4-hydroxy-3,4-dihydrospiro [1-benzopyran-2,1′-cyclopropan]-4-yl}methanesulfonyl)carbamate (1.2 g, 3.1 mmol) in DCM (50 mL) was added TES (1.1 g, 9.3 mmol). After stirring for about 0.5 hours at about 0° C., TFA (2.9 g, 25 mmol) was added to the mixture at this temperature. The mixture was stirred at this temperature for about 3 hours. Water (100 mL) was added and the mixture was extracted with EtOAC (50 mL×2). The combined organic phases were washed with brine, dried, and concentrated in vacuo. The resulting residue was purified by chromatography on silica gel with PE/EtOAc (5/1) to give the intermediate tert-butyl (7-fluorospiro[chroman-2,1′-cyclopropane]-4-ylidene)methylsulfonylcarbamate (MS(ESI): m/z 270 [M+H]⁺). To it was added acetic acid (10 mL) and Pd/C (950 mg). The mixture was stirred under H₂ for about 3 hours. Upon completion, the mixture was filtered, and the filtrate was evaporated in vacuo. To the residue was added DCM (2 mL) and trifluoroacetic acid (2 mL). The reaction was stirred at about room temperature for about 3 hours. Upon completion, the solvent was evaporated in vacuo and the crude product was recrystallized with PE/EtOAc (5/1). Ms=294 [M+Na]⁺.

### Synthesis of (R*)-(7-fluorospiro[chromane-2,1′-cyclopropan]-4-yl)methanesulfonamide (Compound 2) and (S*)-(7-fluorospiro[chromane-2,1′-cyclopropan]-4-yl)methanesulfonamide (Compound 1)

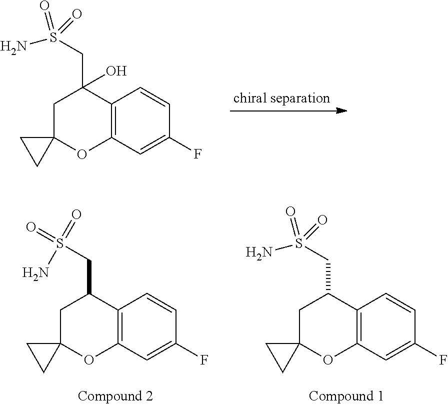 [SMILES: NS(=O)(=O)CC1(O)CC2(CC2)Oc2cc(F)ccc21 ; NS(=O)(=O)C[C@@H]1CC2(CC2)Oc2cc(F)ccc21 ; NS(=O)(=O)C[C@H]1CC2(CC2)Oc2cc(F)ccc21]

The racemic mixture of (7-fluorospiro-[chromane-2,1′-cyclopropan]-4-yl)-methane-sulfonamide was separated by chiral HPLC using the following condition to give the two single enantiomers:

Compound 2 (P1) (retention time: 1.959 min) ¹HNMR (400 MHz, MeOD-d4) δ 7.29˜7.25 (m, 1H), 6.69˜6.64 (m, 1H), 6.48˜6.45 (m, 1H), 3.60˜3.48 (m, 3H), 2.40˜2.36 (m, 1H), 2.25˜2.20 (m, 1H), 1.04˜0.99 (m, 1H), 0.95˜0.86 (m, 2H), 0.67˜0.63 (m, 1H); and

Compound 1 (P2) (retention time: 2.283 min), ¹HNMR (400 MHz, MeOD-d4) δ 7.79˜7.53 (m, 1H), 6.69˜6.64 (m, 1H), 6.48˜6.45 (m, 1H), 3.60˜3.48 (m, 3H), 2.40˜2.36 (m, 1H), 2.25˜2.20 (m, 1H), 1.04˜0.99 (m, 1H), 0.95˜0.86 (m, 2H), 0.67˜0.63 (m, 1H).

### Chiral HPLC Conditions:

| | |
|---|---|
| Instrument: SFC-80 (Thar, | Column: AS 20*250 mm, 10 um |
| Waters) | (Daicel) |
| Column temperature: 35° C. | Mobile phase: CO₂/MeOH(0.2% MeOH |
| | Ammonia) = 80/20 |
| Flow rate: 80 g/min | Back pressure: 100 bar |
| Detection wavelength: 214 nm | Cycle time: 3.8 min |
| Sample solution: 120 mg | Injection volume: 1.9 ml |
| dissolved in 15 ml MeOH | |

### Example 2. Preparation of (R*)-(7-fluorospiro[chromane-2,1′-cyclobutan]-4-yl)methane-sulfonamide (Compound 4) and (S*)-(7-fluorospiro[chromane-2,1′-cyclobutan]-4-yl)methane-sulfonamide (Compound 3)

### Synthesis of 7-fluorospiro [chromane-2,1′-cyclobutan]-4-one

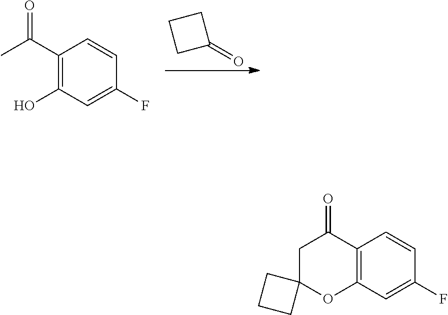

To a solution of 1-(4-fluoro-2-hydroxyphenyl)ethan-1-one (5 g, 32.4 mmol) in toluene (25 mL) was added cyclobutanone (2.95 g, 42.1 mmol) and pyrrolidine (2.30 g, 32.4 mmol). The reaction was stirred at about room temperature for about 1 hour and then refluxed for about 4 hours. The mixture was poured into EtOAc (200 mL) and water (100 mL). The layers were separated, and the organic layer was washed with HCl (aq. 1 M, 100 mL), dried and concentrated. The resulting substance was purified by silica gel column chromatography with a gradient elution of PE/EtOAC (100%:0%) to PE/EtOAc (95%:5%) to provide 7-fluorospiro[chromane-2,1′-cyclobutan]-4-one. MS (ESI): m/z=207 [M+H]⁺.

### Synthesis of tert-butyl (((7-fluoro-4-hydroxyspiro[chromane-2,1′-cyclobutan]-4-yl)methyl)sulfonyl)carbamate

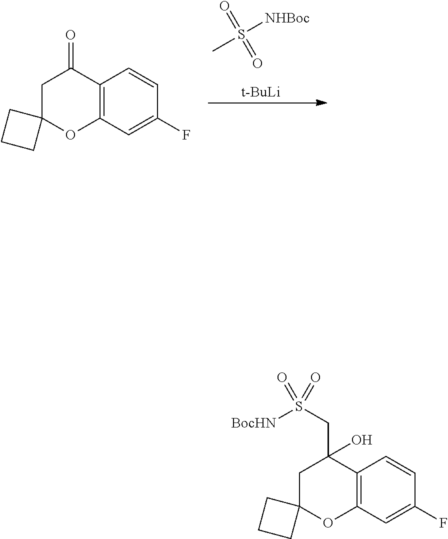 [SMILES: "t-BuLi" ; O=C1CC2(CCC2)Oc2cc(F)ccc21 ; CC(C)(C)OC(=O)NS(C)(=O)=O ; CC(C)(C)OC(=O)NS(=O)(=O)CC1(O)CC2(CCC2)Oc2cc(F)ccc21]

To a solution of tert-butyl N-methanesulfonylcarbamate (4.06 g, 20.8 mmol) in THF (50 mL) was added t-BuLi (1.3 M in pentane) (32 mL, 41.7 mmol) at about −78° C. After stirring at about −78° C. for about 30 min., 7-fluorospiro[chromane-2,1′-cyclobutan]-4-one (2.4 g, 11.6 mmol) in THF (10 mL) was added. The mixture was stirred at about −78° C. for about 2 hours. The mixture was then poured into water (100 mL) and EtOAc (150 mL). Citric acid was slowly added to adjust the pH to 5-6. The layers were separated. The aqueous layer was extracted with EtOAc (100 mL×2), and the combined organic layers were dried and concentrated. The resulting substance was purified by silica gel column chromatography with a gradient elution of PE/EtOAc (100%:0%) to PE/EtOAc (70%:30%) to provide tert-butyl (((7-fluoro-4-hydroxyspiro[chromane-2,1′-cyclobutan]-4-yl)methyl)sulfonyl)carbamate. MS (ESI): m/z=424 [M+Na]⁺.

### Synthesis of (7-fluoro-3,4-dihydrospiro[1-benzopyran-2,1′-cyclobutan]-4-yl)methanesulfonamide

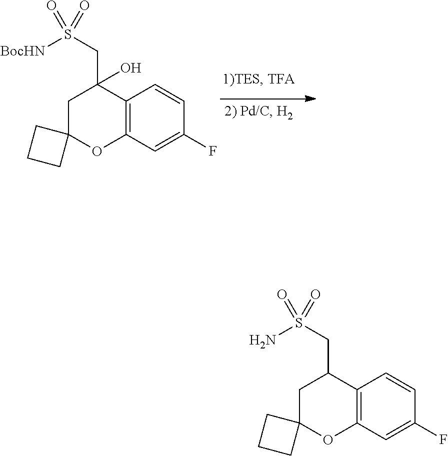 [SMILES: CC(C)(C)OC(=O)NS(=O)(=O)CC1(O)CC2(CCC2)Oc2cc(F)ccc21 ; NS(=O)(=O)CC1CC2(CCC2)Oc2cc(F)ccc21]

To a solution of tert-butyl N-((7-fluoro-4-hydroxy-3,4-dihydrospiro[1-benzopyran-2,1′-cyclobutan]-4-yl)methanesulfonyl)carbamate (3.6 g, 8.96 mmol) in dichloromethane (40 mL) was added TES (2.84 g, 26.8 mmol) at about 0° C. After stirring at about 0° C. for about 30 min., trifluoroacetic acid (12.2 g, 107 mmol) was added. The reaction was stirred at about room temperature for about 16 hours. Solvent was removed. The residue substance was dissolved in methanol (30 mL). To the solution was added Pd/C (10%) (295 mg). The mixture was stirred at about room temperature for about 2 hours under H₂ atmosphere. The mixture was filtered, and the filtrate was concentrated. The resulting substance was purified by silica gel column chromatography with a gradient elution of DCM/MeOH (100%:0%) to DCM/MeOH (97%:3%) to provide {7-fluoro-3,4-dihydrospiro[1-benzopyran-2,1′-cyclobutan]-4-yl}methanesulfonamide. MS (ESI): m/z=286 [M+H]⁺.

### Synthesis of (R*)-(7-fluorospiro[chromane-2,1′-cyclobutan]-4-yl)methane-sulfonamide (Compound 4) and (S*)-(7-fluorospiro[chromane-2,1′-cyclobutan]-4-yl)methane-sulfonamide (Compound 3)

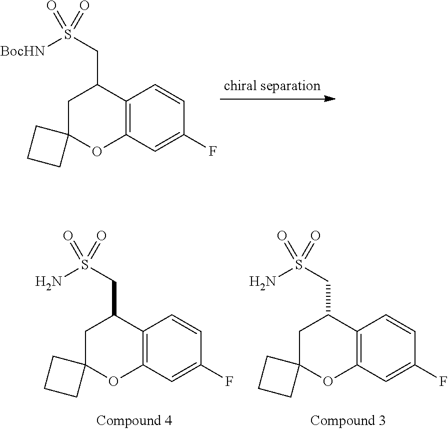

{7-fluoro-3,4-dihydrospiro[1-benzopyran-2,1′-cyclobutan]-4-yl}methane-sulfonamide was separated by chiral HPLC under following conditions to get the two enantiomers:

Compound 4 (P1) (retention time=2.622 min) ¹H NMR (400 MHz, CD₃OD) δ 7.33-7.25 (m, 1H), 6.64 (td, J=8.5, 2.6 Hz, 1H), 6.53 (dd, J=10.3, 2.6 Hz, 1H), 3.75 (dd, J=14.3, 3.1 Hz, 1H), 3.48 (d, J=9.1 Hz, 1H), 3.25 (dd, J=14.3, 9.6 Hz, 1H), 2.65 (dd, J=13.8, 6.0 Hz, 1H), 2.39 (dd, J=20.0, 10.1 Hz, 1H), 2.27-2.08 (m, 3H), 2.01-1.83 (m, 2H), 1.83-1.73 (m, 1H); and Compound 3 (P2) (retention time=3.984 min). ¹H NMR (400 MHz, CD₃OD) δ 7.28 (dd, J=8.1, 6.7 Hz, 1H), 6.64 (td, J=8.5, 2.7 Hz, 1H), 6.53 (dd, J=10.3, 2.6 Hz, 1H), 3.75 (dd, J=14.3, 3.1 Hz, 1H), 3.48 (d, J=8.8 Hz, 1H), 3.25 (dd, J=14.3, 9.6 Hz, 1H), 2.65 (dd, J=13.8, 6.0 Hz, 1H), 2.39 (dd, J=19.9, 10.1 Hz, 1H), 2.26-2.07 (m, 3H), 1.99-1.83 (m, 2H), 1.83-1.72 (m, 1H).

### Chiral HPLC Conditions:

| | |
|---|---|
| Instrument: SFC-80 (Thar, | Column: AD 20 × 250 mm, 10 um |
| Waters) | (Daicel) |
| Column temperature: 35° C. | Mobile phase: CO₂/MeOH(0.2% |
| | MeOH Ammonia) = 80/20 |
| Flow rate: 80 g/min | Back pressure: 100 bar |
| Detection wavelength: 214 nm | Cycle time: 4.6 min |
| Sample solution: 560 mg | Injection volume: 1.9 ml |
| dissolved in 25 ml MeOH | |

### Example 3. Preparation of (R*)-(6,7-difluoro-2,2-dimethylchroman-4-yl)methanesulfonamide (Compound 6) and (S*)-(6,7-difluoro-2,2-dimethylchroman-4-yl)methanesulfonamide (Compound 5)

### Synthesis 6,7-difluoro-2,2-dimethylchroman-4-one

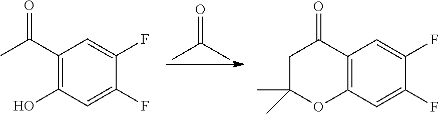

To a solution of 1-(4,5-difluoro-2-hydroxyphenyl)ethan-1-one (14.4 g, 83.6 mmol) in MeOH (150 mL) was added pyrrolidine (5.94 g, 83.6 mmol) and propan-2-one (9.69 g, 167 mmol). The mixture was stirred at about room temperature for about 16 hours. Solvent was removed and the residue was poured into water (100 mL). The mixture was extracted with DCM (3×100 mL). The combined organics were dried and concentrated. The resulting substance was purified by silica gel column chromatography with a gradient elution of PE/EtOAc (100%:0%) to PE/EtOAc (90%:10%) to provide 6,7-difluoro-2,2-dimethylchroman-4-one.

### Synthesis of tert-butyl (((6,7-difluoro-4-hydroxy-2,2-dimethylchroman-4-yl)methyl)sulfonyl)carbamate

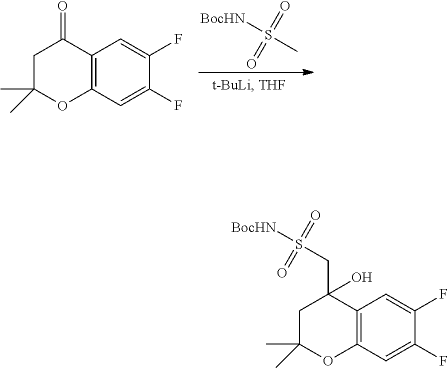 [SMILES: CC1(C)CC(=O)c2cc(F)c(F)cc2O1 ; CC(C)(C)OC(=O)NS(C)(=O)=O ; CC(C)(C)OC(=O)NS(=O)(=O)CC1(O)CC(C)(C)Oc2cc(F)c(F)cc21]

To a solution of tert-butyl N-methanesulfonylcarbamate (3.41 g, 17.5 mmol) in THF (150 mL) was added t-BuLi (1.3 M in pentane) (27 mL 35.1 mmol) at about −78° C. After stirring at about −78° C. for about 30 min., 6,7-difluoro-2,2-dimethylchroman-4-one (2.5 g, 11.7 mmol) in THF (20 mL) was added. The mixture was stirred at about −78° C. for about 30 min. The mixture was allowed to gradually warm to about 0° C. over a period of about 2 hours. Water (100 mL) was added to quench the reaction and citric acid was added to adjust the pH to 5-6. The mixture was extracted with EtOAc (200 mL×2), dried and concentrated. The resulting substance was purified by silica gel column chromatography with a gradient elution of PE/EtOAc (100%: 0%) to PE/EtOAc (75%:25%) to provide tert-butyl (((6, 7-difluoro-4-hydroxy-2,2-dimethylchroman-4-yl)methyl)sulfonyl)carbamate. MS (ESI): m/z=430 [M+Na]⁺.

### Synthesis of (6,7-difluoro-2,2-dimethylchroman-4-yl)methanesulfonamide

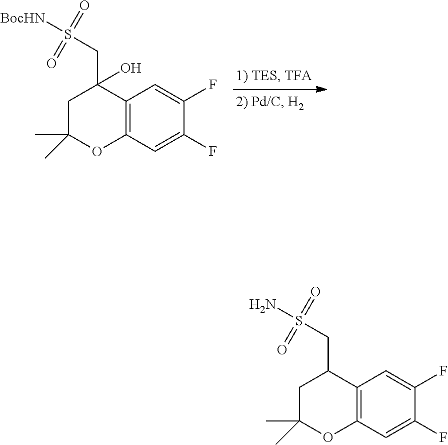 [SMILES: CC(C)(C)OC(=O)NS(=O)(=O)CC1(O)CC(C)(C)Oc2cc(F)c(F)cc21 ; CC1(C)CC(CS(N)(=O)=O)c2cc(F)c(F)cc2O1]

To a solution of tert-butyl (((6,7-difluoro-4-hydroxy-2,2-dimethylchroman-4-yl)methyl)sulfonyl)carbamate (9 g, 22.0 mmol) in DCM (80 mL) was added TES (7.67 g, 66.0 mmol) at 0° C. The mixture was stirred at about 0° C. for about 20 min and TFA (20.0 g, 176 mmol) was added. The mixture was stirred at about room temperature for about 16 hours. The solvent was removed. The residue was dissolved in MeOH (200 mL) and Pd/C (10%) (1.10 g) was added. The reaction mixture was stirred at about room temperature for about 2 hours under H₂ atmosphere. The mixture was filtered and the filtrate was concentrated. The residue substance was washed with EtOAc (15 mL), heptane (90 mL) and then recrystallized in acetone and water to provide (6,7-difluoro-2,2-dimethylchroman-4-yl)methanesulfonamide. MS (ESI): m/z=292 [M+H]⁺.

### Synthesis of (R*)-(6,7-difluoro-2,2-dimethylchroman-4-yl)methanesulfonamide (Compound 6) and (S*)-(6,7-difluoro-2,2-dimethylchroman-4-yl)methane-sulfonamide (Compound 5)

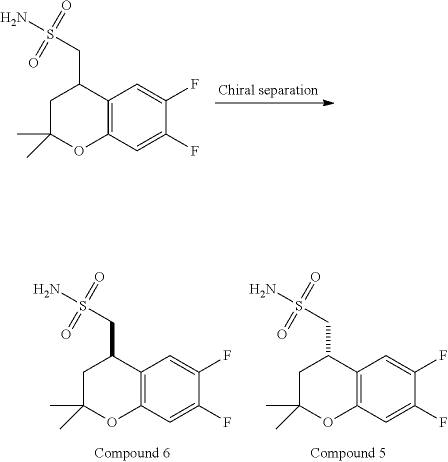

(6,7-difluoro-2,2-dimethylchroman-4-yl)methanesulfonamide was separated by Chiral HPLC under the following conditions to provide two enantiomers.

Compound 6 (P1) (retention time=0.985 min) MS (ESI): m/z=292[M+H]⁺. ¹H NMR (400 MHz, CD₃OD) δ 7.32-7.27 (m, 1H), 6.65-6.61 (dd, J=11.8, 7.2 Hz, 1H), 3.76-3.71 (dd, J=14.4, 3.2 Hz, 1H), 3.46-3.44 (m, 1H), 3.25-3.21 (dd, J=14.4, 8.8 Hz, 1H), 2.42-2.37 (dd, J=13.8, 6.4 Hz, 1H), 1.77-1.71 (dd, J=13.8, 11.3 Hz, 1H), 1.42 (s, 3H), 1.27 (s, 3H); and Compound 5 (P2) (retention time=1.226 min) MS (ESI): m/z=292[M+H]⁺. ¹H NMR (400 MHz, CD₃OD) δ 7.32-7.27 (dd, J=11.6, 9.2 Hz, 1H), 6.65-6.61 (dd, J=11.8, 7.2 Hz, 1H), 3.76-3.71 (dd, J=14.4, 3.2 Hz, 1H), 3.46-3.44 (d, J=8.6 Hz, 1H), 3.25-3.2 (dd, J=14.4, 8.8 Hz, 1H), 2.34-2.32 (dd, J=13.8, 6.4 Hz, 1H), 1.77-1.71 (dd, J=13.8, 11.2 Hz, 1H), 1.42 (s, 3H), 1.27 (s, 3H).

### Chiral HPLC Conditions:

| | |
|---|---|
| Instrument: SFC-80 (Thar, Waters) | Column: AD 20 × 250 mm, 10 um |
| | (Daicel) |
| Column temperature: 35° C. | |
| Mobile phase: CO₂/MeOH (0.2% | |
| MeOH Ammonia) = 85/15 | |
| Flow rate: 80 g/min | Back pressure: 100 bar |
| Detection wavelength: 214 nm | Cycle time: 3.9 min |
| Sample solution: 3000 mg dissolved | Methanol Injection volume: |
| in 36 mL | 0.6 mL |

### Example 4. Preparation of ((3S*,4R*)-6,7-difluoro-3-methylchroman-4-yl)methanesulfonamide (Compound 8), ((3R*,4S*)-6,7-difluoro-3-methylchroman-4-yl)methanesulfonamide (Compound 7), ((3S*,4S*)-6,7-difluoro-3-methylchroman-4-yl)methanesulfonamide (Compound 9) and ((3R*,4R*)-6,7-difluoro-3-methylchroman-4-yl)methanesulfonamide (Compound 10)

### Synthesis of 6,7-difluoro-3-methylchroman-4-one

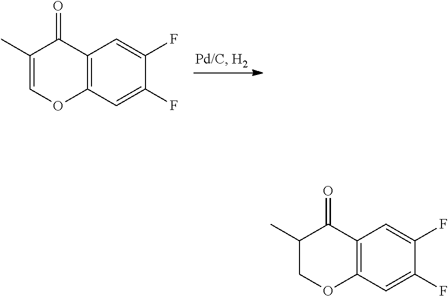

To a solution of 6,7-difluoro-3-methyl-4H-chromen-4-one (5.9 g, 28.5 mmol) in EtOAc (200 mL) was added TEA (4.32 g, 42.7 mmol) and Pd/C (1.18 g, 10% wet). The reaction was stirred at about room temperature for about 16 hours under Hz. The mixture was filtered, and filtrate concentrated. The residue was purified by silica gel column chromatography with an isocratic elution of PE (85%) and EtOAc (15%) to provide 6,7-difluoro-3-methylchroman-4-one. MS (ESI): m/z=199[M+H]⁺.

### Synthesis of tert-butyl (((6,7-difluoro-4-hydroxy-3-methylchroman-4-yl)methyl)sulfonyl)carbamate

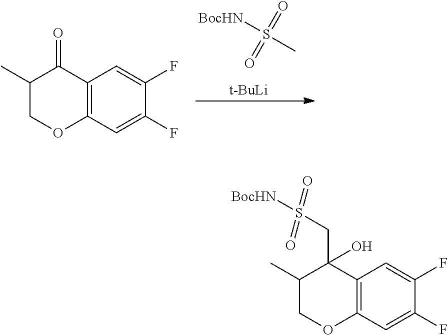 [SMILES: "t-BuLi" ; CC1COc2cc(F)c(F)cc2C1=O ; CC(C)(C)OC(=O)NS(C)(=O)=O ; CC1COc2cc(F)c(F)cc2C1(O)CS(=O)(=O)NC(=O)OC(C)(C)C]

To a solution of 6,7-difluoro-3-methylchroman-4-one (4.8 g, 23.0 mmol) in THF (90 mL) was added t-BuLi (1.3M in pentane, 35 mL, 69.0 mmol). The reaction was stirred at about −78° C. for 2 h. Saturated aqueous NH₄Cl (20 mL) was added to the reaction vessel and the resulting biphasic mixture was transferred to a separatory funnel. The layers were separated, and the aqueous phase was extracted with EtOAc (3×40 mL). The combined organics were dried and concentrated in vacuo to provide tert-butyl (((6,7-difluoro-4-hydroxy-3-methylchroman-4-yl)methyl)sulfonyl)carbamate. MS (ESI): m/z=319.0[M−56-18]⁺.

### Synthesis of (6,7-difluoro-3-methylchroman-4-yl)methanesulfonamide

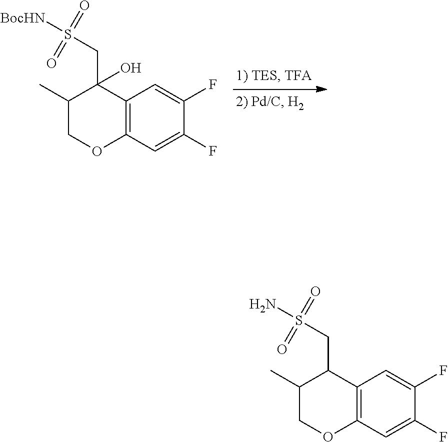 [SMILES: CC1COc2cc(F)c(F)cc2C1(O)CS(=O)(=O)NC(=O)OC(C)(C)C ; CC1COc2cc(F)c(F)cc2C1CS(N)(=O)=O]

To a solution of tert-butyl (((6,7-difluoro-4-hydroxy-3-methylchroman-4-yl)methyl)sulfonyl)carbamate (5 g, 7.62 mmol) in DCM (3 mL) was added TES (2.65 g, 22.8 mmol) and TFA (8.68 g, 76.2 mmol). The reaction was stirred at about room temperature for about 16 hours and then solvent removed. The residue was purified by silica gel column chromatography with an isocratic elution of DCM/MeOH (95%:5%) to provide the intermediate (6,7-difluoro-3-methyl-2H-chromen-4-yl)methanesulfonamide. MS (ESI): m/z=298.0 [M+Na]⁺. (6,7-Difluoro-3-methyl-2H-chromen-4-yl)methanesulfonamide (5 g, 10.8 mmol) was dissolved in MeOH (15 mL) and Pd/C (1.41 g, 10% wet) was added. The reaction was stirred at about room temperature under H₂ for about 2 hours. After filtration, the filtrate was concentrated and the residue was purified by silica gel column chromatography with a gradient elution of DCM/MeOH (100%:0%) to DCM/MeOH (50%:50%) to provide (6,7-difluoro-3-methylchroman-4-yl)methanesulfonamide as a mixture of 4 diastereomers. MS (ESI): m/z=300.1[M+Na]⁺.

### Synthesis of ((3S*,4R*)-6,7-difluoro-3-methylchroman-4-yl)methane-sulfonamide (Compound 8), ((3R*,4S*)-6,7-difluoro-3-methylchroman-4-yl)methanesulfonamide (Compound 7), ((3S*,4S*)-6,7-difluoro-3-methylchroman-4-yl)methanesulfonamide (Compound 9) and ((3R*, 4R*)-6,7-difluoro-3-methylchroman-4-yl)methanesulfonamide (Compound 10)

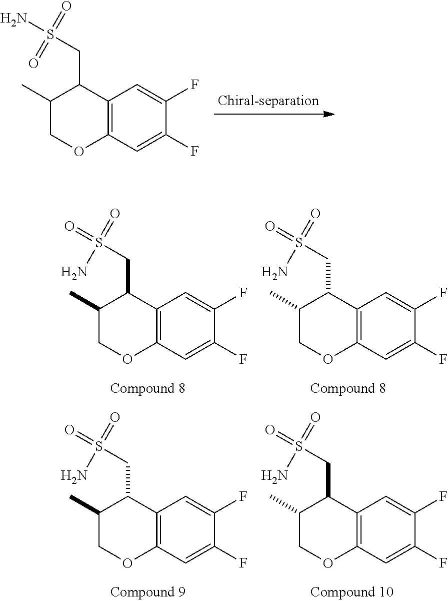

The diastereomeric mixture of (6,7-difluoro-3-methylchroman-4-yl)methanesulfonamide was separated by chiral HPLC under the following conditions to give the 4 diastereomers.

Compound 8 (P1) (retention time=1.776 min). MS (ESI): m/z=300[M+Na]⁺. ¹H NMR (400 MHz, CD₃OD) δ 7.08 (dd, J=11.1, 9.0 Hz, 1H), 6.56 (dd, J=11.8, 7.1 Hz, 1H), 4.03 (dd, J=11.4, 2.3 Hz, 1H), 3.87 (ddd, J=11.4, 3.5, 1.3 Hz, 1H), 3.32 (dd, J=14.7, 8.5 Hz, 1H), 3.26-3.22 (m, 1H), 2.98 (t, J=18.8 Hz, 1H), 2.30 (qd, J=6.9, 3.8 Hz, 1H), 0.97 (d, J=7.0 Hz, 3H); and Compound 7 (P2) (retention time=2.349 min). MS (ESI): m/z=300.1[M+Na]⁺. ¹H NMR (400 MHz, CD₃OD) δ 7.08 (dd, J=11.1, 9.0 Hz, 1H), 6.57 (dd, J=11.8, 7.1 Hz, 1H), 4.03 (dd, J=11.4, 2.3 Hz, 1H), 3.87 (ddd, J=11.4, 3.5, 1.3 Hz, 1H), 3.32 (dd, J=14.7, 8.5 Hz, 1H), 3.28-3.22 (m, 1H), 2.95 (d, J=8.1 Hz, 1H), 2.30 (qd, J=7.0, 4.0 Hz, 1H), 1.23-0.76 (m, 3H); and Compound 10 (P3) (retention time=2.768 min). MS (ESI): m/z=300.1[M+Na]⁺, ¹H NMR (400 MHz, CD₃OD) δ 7.27 (dd, J=11.6, 9.1 Hz, 1H), 6.59 (dt, J=77.7, 38.8 Hz, 1H), 4.13 (dd, J=11.0, 2.9 Hz, 1H), 3.91 (dd, J=11.0, 7.3 Hz, 1H), 3.52-3.38 (m, 1H), 3.33-3.19 (m, 1H), 2.47-2.29 (m, 1H), 1.05 (d, J=7.0 Hz, 3H); and Compound 9 (P4) (retention time=3.890 min). MS (ESI): m/z=300.1[M+Na]⁺. ¹H NMR (400 MHz, CD₃OD) δ 7.18 (dd, J=11.6, 9.2 Hz, 1H), 6.54 (dd, J=11.8, 7.2 Hz, 1H), 4.03 (dd, J=11.0, 2.4 Hz, 1H), 3.82 (dd, J=11.0, 7.3 Hz, 1H), 3.36 (dd, J=10.6, 5.3 Hz, 1H), 3.27-3.21 (m, 2H), 2.45-2.11 (m, 1H), 1.30-0.63 (m, 3H).

### Chiral HPLC Conditions:

| | |
|---|---|
| Instrument: SFC-80 (Thar, Waters) | Column: IG 20*250 mm, 10 um |
| | (Daicel) |
| Column temperature: 35° C. | Mobile phase: CO₂/MeOH(0.2% |
| | MeOH Ammonia) = 80/20 |
| Flow rate: 80 g/min | Back pressure: 100 bar |
| Detection wavelength: 214 nm | Cycle time: 4.3 min |
| Sample solution: 2000 mg | |
| dissolved in 45 ml MeOH | |
| Injection volume: 0.6 ml | |

### Example 5. Preparation of ((2S*,4S*)-2-ethyl-7-fluorochroman-4-yl)methanesulfonamide (Compound 13), ((2R*,4R*)-2-ethyl-7-fluorochroman-4-yl)methanesulfonamide (Compound 14), ((2S*,4R*)-2-ethyl-7-fluorochroman-4-yl)methanesulfonamide (Compound 12) and ((2R*,4S*)-2-ethyl-7-fluorochroman-4-yl)methanesulfonamide (Compound 11)

### Synthesis of 2-ethyl-7-fluorochroman-4-one

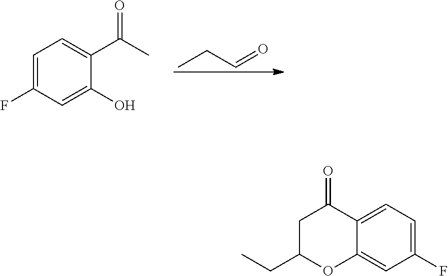

To a solution of 1-(4-fluoro-2-hydroxyphenyl)ethanone (15 g, 97.3 mmol) in EtOH (200 mL) was added DIPEA (13.8 g, 107 mmol) and propanal (6.21 g, 107 mmol). The reaction mixture was heated to about 140° C. in a microwave and stirred at that temperature for about 1 hour. EtOAc (50 mL) was added to the reaction vessel and the resulting biphasic mixture was transferred to a separatory funnel. The layers were separated, and the organic phase was washed with saturated sodium chloride solution (aq. 2×25 mL). The organic phase was dried over anhydrous Na₂SO₄, filtered and concentrated in vacuo. The resulting substance was purified by silica gel column chromatography with a gradient elution of PE/EtOAc (100%:0%) to PE/EtOAc (90%:10%) to provide 2-ethyl-7-fluorochroman-4-one. MS (ESI): m/z=195 [M+H]⁺.

### Synthesis of tert-butyl (2-ethyl-7-fluoro-4-hydroxychroman-4-yl)methylsulfonylcarbamate

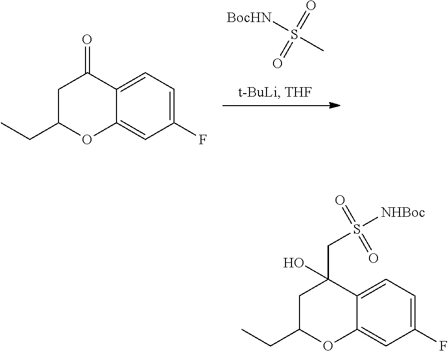 [SMILES: CCC1CC(=O)c2ccc(F)cc2O1 ; CC(C)(C)OC(=O)NS(C)(=O)=O ; CCC1CC(O)(CS(=O)(=O)NC(=O)OC(C)(C)C)c2ccc(F)cc2O1]

To a solution of tert-butyl N-methanesulfonylcarbamate (2.98 g, 15.3 mmol) in THF (100 mL) was added t-BuLi (1.3 M in pentane, 23.5 mL, 30.6 mmol) at about −78° C. The reaction mixture was stirred at that temperature for about 1 hour. 2-ethyl-7-fluorochroman-4-one (2.3 g, 11.8 mmol) in THF (2 mL) was added to the reaction. The reaction was stirred at about room temperature for about 16 hours. Water (100 mL) and NaOH (aq. 1M, 20 mL) was added to the reaction vessel and the resulting biphasic mixture was transferred to a separatory funnel. The layers were separated and the organic phase was washed with saturated aqueous NaCl (2×20 mL). The organic phase was dried over anhydrous Na₂SO₄, filtered and concentrated in vacuo. The resulting substance was purified by silica gel column chromatography with a gradient elution of PE/EtOAc (100%:0%) to PE/EtOAc (80%:20%) to provide tert-butyl (2-ethyl-7-fluoro-4-hydroxychroman-4-yl)methylsulfonylcarbamate. MS (ESI): m/z=412 [M+Na]⁺.

### Synthesis of (2-ethyl-7-fluorochroman-4-yl)methanesulfonamide

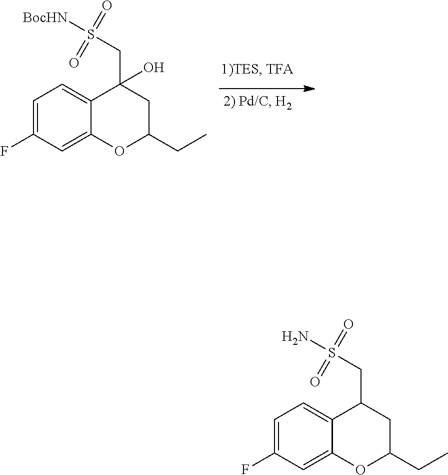 [SMILES: CCC1CC(O)(CS(=O)(=O)NC(=O)OC(C)(C)C)c2ccc(F)cc2O1 ; CCC1CC(CS(N)(=O)=O)c2ccc(F)cc2O1]

To a solution of tert-butyl (2-ethyl-7-fluoro-4-hydroxychroman-4-yl)methylsulfonylcarbamate (4.6 g, 9.6 mmol) in DCM (20 mL) was added TES (3.3 g, 28.6 mmol) and TFA (8.71 g, 76.4 mmol). The reaction mixture was stirred at about room temperature for 16 h and was concentrated in vacuo. The resulting residue was dissolved in EtOAc (50 mL) and Pd/C (1 g, 0.94 mmol) was added. The reaction was stirred at about room temperature under H₂ for about 2 hours. After filtration, the filtrate was concentrated in vacuo. LCMS indicated that two pairs of enantiomers of (2-ethyl-7-fluorochroman-4-yl)methanesulfonamide with a diastereomeric ratio of 20:1 was produced. The crude product was washed with heptane (20 mL) to afford a diastereomeric mixture of (2-ethyl-7-fluorochroman-4-yl)methanesulfonamide. MS (ESI): m/z=274 [M+H]⁺.

### Synthesis of ((2S*,4S*)-2-ethyl-7-fluorochroman-4-yl)methanesulfonamide (Compound 13), ((2R*,4R*)-2-ethyl-7-fluorochroman-4-yl)methanesulfonamide (Compound 14), ((2S*,4R*)-2-ethyl-7-fluorochroman-4-yl)methanesulfonamide (Compound 12) and ((2R*,4S*)-2-ethyl-7-fluorochroman-4-yl)methanesulfonamide (Compound 11)

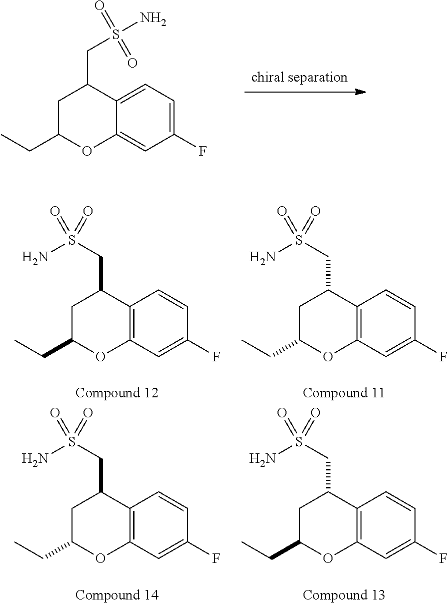

The diastereomeric mixture of (2-ethyl-7-fluorochroman-4-yl)methanesulfonamide mixture (2 g) was separated by chiral HPLC under the following conditions to provide two enantiomers. The minor pair of enantiomers were not isolated from the mixture.

Compound 12 (P1) (retention time=1.468 min). MS (ESI): m/z=274 [M+H]⁺. ¹H NMR (500 MHz, CD₃OD) δ 7.35-7.25 (m, 1H), 6.68-6.60 (m, 1H), 6.55-6.47 (m, 1H), 3.99-3.88 (m, 1H), 3.79 (dd, J=14.3, 3.0 Hz, 1H), 3.58-3.46 (m, 1H), 3.16 (dd, J=14.3, 9.4 Hz, 1H), 2.59 (dd, J=13.3, 5.9 Hz, 1H), 1.84-1.65 (m, 2H), 1.59 (dd, J=25.0, 11.5 Hz, 1H), 1.08 (t, J=7.5 Hz, 3H); and Compound 11 (P2) (retention time=1.887 min). MS (ESI): m/z=274 [M+H]⁺. ¹H NMR (500 MHz, CD₃OD) δ 7.31 (dd, J=8.1, 7.0 Hz, 1H), 6.70-6.58 (m, 1H), 6.52 (dd, J=10.3, 2.7 Hz, 1H), 4.01-3.87 (m, 1H), 3.79 (dd, J=14.3, 3.0 Hz, 1H), 3.62-3.42 (m, 1H), 3.16 (dd, J=14.3, 9.4 Hz, 1H), 2.65-2.51 (m, 1H), 1.84-1.66 (m, 2H), 1.66-1.52 (m, 1H), 1.08 (t, J=7.5 Hz, 3H).

### Chiral HPLC Conditions

| | |
|---|---|
| Instrument: SFC-80 (Thar, Waters) | Column: AD 20 × 250 mm, 10 um |
| | (Daicel) |
| Column temperature: 35° C. | Mobile phase: CO₂/EtOH (0.5% |
| | MeOH Ammonia) = 85/15 |
| Flow rate: 80 g/min | Back pressure: 100 bar |
| Detection wavelength: 214 nm | Cycle time: 4.2 min |
| Sample solution: 2000 mg | |
| dissolved in 40 mL MeOH | |
| Injection volume: 0.6 mL. | |

### Example 6. Preparation of (R*)-(7,8-difluorochroman-4-yl)methanesulfonamide and (Compound 16) and (S*)-(7,8-difluorochroman-4-yl)methanesulfonamide (Compound 15)

### Synthesis of tert-butyl (((7,8-difluoro-4-hydroxychroman-4-yl)methyl)-sulfonyl)carbamate

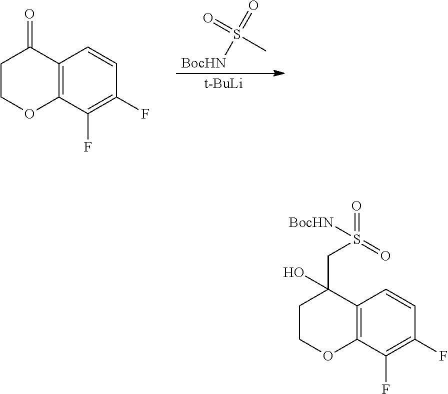 [SMILES: "t-BuLi" ; O=C1CCOc2c1ccc(F)c2F ; CC(C)(C)OC(=O)NS(C)(=O)=O ; CC(C)(C)OC(=O)NS(=O)(=O)CC1(O)CCOc2c1ccc(F)c2F]

To a solution of tert-butyl N-methanesulfonylcarbamate (2 g, 10.2 mmol) in THF (30 mL) was added t-BuLi (1.3 M in pentane, 1.56 mL, 20.4 mmol) at about −78° C. After stirring at about −78° C. for about 0.5 hours, 7,8-difluorochroman-4-one (939 mg, 5.09 mmol) in THF (5 mL) was added. The reaction was stirred at about −78° C. for about 1.5 hours. Upon completion, water (100 mL) was added to quench the reaction and citric acid was added to adjust the pH to around 6. The mixture was extracted with EtOAc (200 mL×2) and the combined organic layers were dried and concentrated. The resulting substance was purified by silica gel column chromatography with a gradient elution of DCM/MeOH (100%:0%) to DCM/MeOH (95%:5%) to provide tert-butyl (((7,8-difluoro-4-hydroxychroman-4-yl)methyl)sulfonyl)carbamate. MS (ESI): m/z=402 [M+Na]⁺.

### Synthesis of (7,8-difluorochroman-4-yl)methanesulfonamide

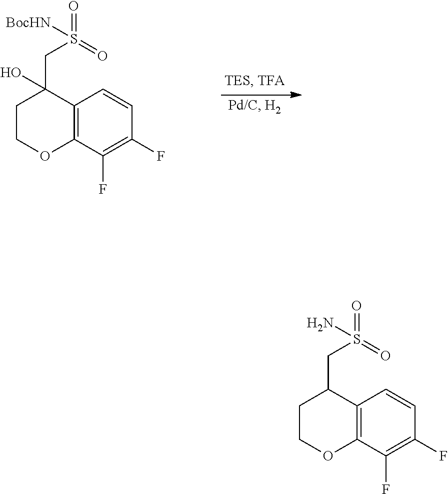 [SMILES: CC(C)(C)OC(=O)NS(=O)(=O)CC1(O)CCOc2c1ccc(F)c2F ; "Pd/C, H2" ; NS(=O)(=O)CC1CCOc2c1ccc(F)c2F]

To a solution of tert-butyl (((7,8-difluoro-4-hydroxychroman-4-yl)methyl)-sulfonyl)carbamate (1.94 g, 5.11 mmol) in DCM (100 mL) was added TES (1.77 g, 15.3 mmol) at about 0° C. After stirring at about 0° C. for about 30 min., TFA (7.02 g, 61.3 mmol) was added. The reaction mixture was stirred at about room temperature for about 16 hours. Upon completion, the solvent was removed in vacuo. The residue was dissolved in methanol (10 mL) and Pd/C (10%) (107 mg) was added. The mixture was stirred at about room temperature for about 2 hours under H₂. Upon completion, the mixture was filtered and the filtrate was concentrated. The residue was triturated with PE/EtOAc (4:1, 100 mL) and then filtered to provide (7,8-difluorochroman-4-yl)methanesulfonamide. MS (ESI): m/z=286 [M+Na]⁺.

### Synthesis of (R*)-(7,8-difluorochroman-4-yl)methanesulfonamide and (Compound 6) and (S*)-(7,8-difluorochroman-4-yl)methanesulfonamide (Compound 15)

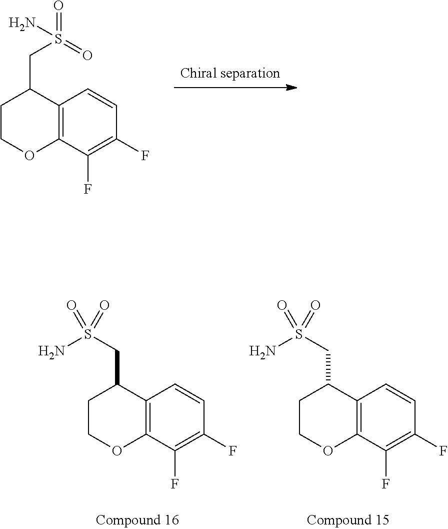 [SMILES: NS(=O)(=O)CC1CCOc2c1ccc(F)c2F ; NS(=O)(=O)C[C@@H]1CCOc2c1ccc(F)c2F ; NS(=O)(=O)C[C@H]1CCOc2c1ccc(F)c2F]

The racemic mixture of (7,8-difluorochroman-4-yl)methanesulfonamide (850 mg) was separated by chiral HPLC under the following conditions to give the two enantiomers.

Compound 16 (retention time=1.01 min), MS (ESI): m/z 286 [M+Na]⁺, ¹H NMR (500 MHz, CD₃OD): δ 7.04-7.00 (m, 1H), 6.80-6.75 (m, 1H), 4.38-4.34 (m, 1H), 4.28-4.23 (m, 1H), 3.51-3.48 (m, 1H), 3.46-3.38 (m, 2H), 2.37-2.33 (m, 1H), 2.28-2.23 (m, 1H); and Compound 15 (retention time=1.62 min), MS (ESI): m/z 286 [M+Na]⁺, ¹H NMR (500 MHz, CD₃OD): δ 7.04-7.00 (m, 1H), 6.80-6.75 (m, 1H), 4.38-4.34 (m, 1H), 4.28-4.23 (m, 1H), 3.51-3.48 (m, 1H), 3.46-3.38 (m, 2H), 2.37-2.33 (m, 1H), 2.28-2.22 (m, 1H).

### Chiral HPLC Conditions:

| | |
|---|---|
| Instrument: SFC-80 (Thar, Waters) | Column: AY 20 × 250 mm, 10 um |
| | (Daicel) |
| Column temperature: 35° C. | Mobile phase: CO₂/MeOH (0.2% |
| | MeOH Ammonia) = 70/30 |
| Flow rate: 80 g/min | Back pressure: 100 bar |
| Detection wavelength: 214 nm | Cycle time: 3.0 min |
| Sample solution: 850 mg | |
| dissolved in 20 mL MeOH | |
| Injection volume: 1.0 mL | |

### Example 7. Preparation of (R*)-(5,7-difluorochroman-4-yl)methanesulfonamide (Compound 18) and (S*)-(5,7-difluorochroman-4-yl)methanesulfonamide (Compound 17)

### Synthesis of tert-butyl (((5,7-difluoro-4-hydroxychroman-4-yl)methyl)-sulfonyl)carbamate

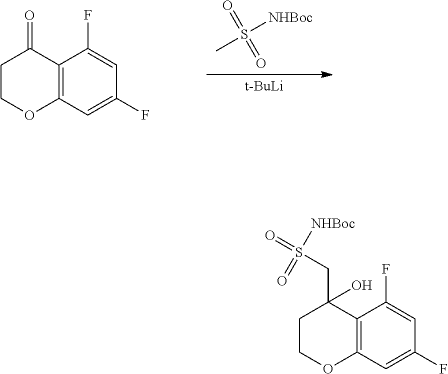 [SMILES: "t-BuLi" ; O=C1CCOc2cc(F)cc(F)c21 ; CC(C)(C)OC(=O)NS(C)(=O)=O ; CC(C)(C)OC(=O)NS(=O)(=O)CC1(O)CCOc2cc(F)cc(F)c21]

To a solution of tert-butyl (methylsulfonyl)carbamate (1.71 g, 8.78 mmol) in THF (15 mL) was added t-BuLi (1.3 M in pentane, 13.4 mL, 17.5 mmol) at about −78° C. After stirring at about −78° C. for about 1 hour, 5,7-difluorochroman-4-one (0.9 g, 4.88 mmol) in THF (5 mL) was added. The reaction was stirred at about −78° C. for about 2 hours. Upon completion, water (80 mL) was added to quench the reaction and citric acid was added to adjust the pH to 5-6. The mixture was extracted with EtOAc (100 mL×2), and the combined organic layers were dried and concentrated. The resulting substance was purified by silica gel column chromatography with a gradient elution of PE/EtOAc (100%:0%) to PE/EtOAc (75%:25%) to provide tert-butyl (((5,7-difluoro-4-hydroxychroman-4-yl)methyl)sulfonyl)carbamate. MS (ESI): m/z=402 [M+Na]⁺.

### Synthesis of (5,7-difluorochroman-4-yl)methanesulfonamide

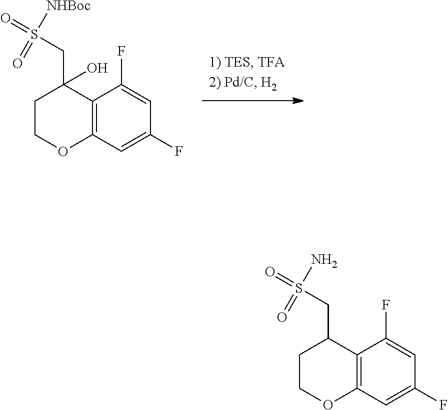 [SMILES: CC(C)(C)OC(=O)NS(=O)(=O)CC1(O)CCOc2cc(F)cc(F)c21 ; NS(=O)(=O)CC1CCOc2cc(F)cc(F)c21]

To a solution of tert-butyl (((5,7-difluoro-4-hydroxychroman-4-yl)methyl)-sulfonyl)carbamate (2 g, 5.27 mmol) in DCM (20 mL) was added TES (1.83 g, 15.8 mmol) at about 0° C. After stirring at about 0° C. for about 30 min., TFA (7.20 g, 63.2 mmol) was added and the reaction was stirred at about room temperature for about 16 hours. Upon completion, the solvent was removed, and the residue was dissolved in MeOH (20 mL). To the solution was added Pd/C (10%) (439 mg) and the mixture was stirred at about room temperature for about 2 hours under H₂ atmosphere. Upon completion, the mixture was filtered, and filtrate concentrated. The residue was washed with EtOAc/heptane (1:5) and then recrystallized from acetone and water to give (5,7-difluorochroman-4-yl)methanesulfonamide. MS (ESI): m/z=264 [M+H]⁺.

### Synthesis of (R*)-(5,7-difluorochroman-4-yl)methanesulfonamide (Compound 18) and (S*)-(5,7-difluorochroman-4-yl)methanesulfonamide (Compound 17)

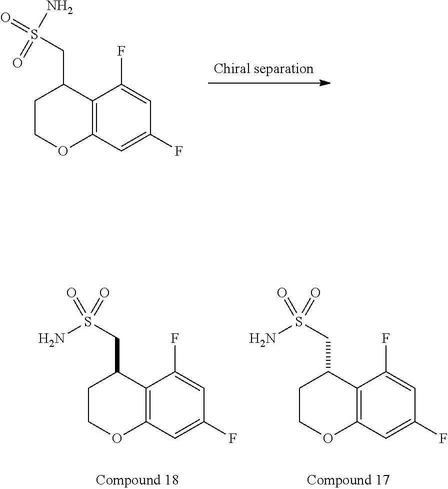 [SMILES: NS(=O)(=O)CC1CCOc2cc(F)cc(F)c21 ; NS(=O)(=O)C[C@@H]1CCOc2cc(F)cc(F)c21 ; NS(=O)(=O)C[C@H]1CCOc2cc(F)cc(F)c21]

(5,7-difluorochroman-4-yl)methanesulfonamide was separated by chiral HPLC using the following conditions to provide the two enantiomers.

Compound 18 (P1) (retention time=1.24 min) MS (ESI): m/z=264 [M+H]⁺. ¹H NMR (400 MHz, CD₃OD) δ 6.52 (ddd, J=10.2, 9.2, 2.5 Hz, 1H), 6.46-6.40 (m, 1H), 4.34 (dt, J=11.6, 2.9 Hz, 1H), 4.23-4.13 (m, 1H), 3.64 (d, J=4.7 Hz, 1H), 3.39 (dd, J=18.7, 8.9 Hz, 2H), 2.59-2.50 (m, 1H), 2.11-1.99 (m, 1H); and Compound 17 (P2) (retention time=1.59 min) MS (ESI): m/z=264 [M+H]⁺. ¹H NMR (400 MHz, CD₃OD) δ 6.56-6.48 (m, 1H), 6.46-6.39 (m, 1H), 4.34 (dt, J=11.5, 2.9 Hz, 1H), 4.24-4.13 (m, 1H), 3.64 (s, 1H), 3.39 (dd, J=18.8, 8.9 Hz, 2H), 2.55 (dd, J=14.7, 2.3 Hz, 1H), 2.16-1.97 (m, 1H).

### Chiral HPLC Conditions:

| | |
|---|---|
| Instrument: SFC-80 (Thar, Waters) | Column: AY 20 × 250 mm, 10 um |
| | (Daicel) |
| Column temperature: 35° C. | Mobile phase: CO₂/MeOH (0.2% |
| | MeOH Ammonia) = 85/15 |
| Flow rate: 80 g/min | Back pressure: 100 bar Detection |
| | wavelength: 214 nm |
| Cycle time: 2.6 min | Sample solution: 700 mg dissolved |
| | in 30 mL MeOH |
| Injection volume: 0.6 mL | |

### Example 8. Preparation of ((3S*,4R*)-3-ethyl-7-fluorochroman-4-yl)methanesulfonamide (Compound 20), ((3R*,4S*)-3-ethyl-7-fluorochroman-4-yl)methanesulfonamide (Compound 19), ((3S*,4S*)-3-ethyl-7-fluorochroman-4-yl)methanesulfonamide (Compound 22) and ((3R*,4R*)-3-ethyl-7-fluorochroman-4-yl)methanesulfonamide (Compound 21)

### Synthesis of tert-butyl (3-ethyl-7-fluoro-4-hydroxychroman-4-yl)methylsulfonylcarbamate

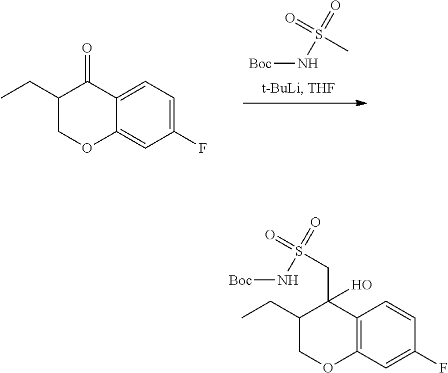 [SMILES: CCC1COc2cc(F)ccc2C1=O ; CC(C)(C)OC(=O)NS(C)(=O)=O ; CCC1COc2cc(F)ccc2C1(O)CS(=O)(=O)NC(=O)OC(C)(C)C]

To a solution of tert-butyl methylsulfonylcarbamate (9.9 g, 50.9 mmol) in THF (150 mL) was added t-BuLi (1.3 M in pentane, 78 mL, 101 mmo.) at about −78° C. under nitrogen. After stirring for about 1.0 hour at this temperature, a solution of 3-ethyl-7-fluorochroman-4-one (9.0 g, 46.3 mmol) in THF (30 mL) was added and the mixture was stirred at about −78° C. for an about additional 2 hours. Upon completion, the reaction was quenched with water (300 mL), acidified with sat. critic acid (aq.) till pH˜4. The mixture was extracted with ethyl acetate (200 mL×3), and the combined organic layers were dried and concentrated. The resulting substance was purified by silica gel column eluted with PE/EtOAc (3/1) to PE/EtOAc (1:1) to give tert-butyl (3-ethyl-7-fluoro-4-hydroxychroman-4-yl)methylsulfonylcarbamate. MS (ESI): m/z 407 [M+NH₄]⁺.

### Synthesis of (3-ethyl-7-fluorochroman-4-yl)methanesulfonamide

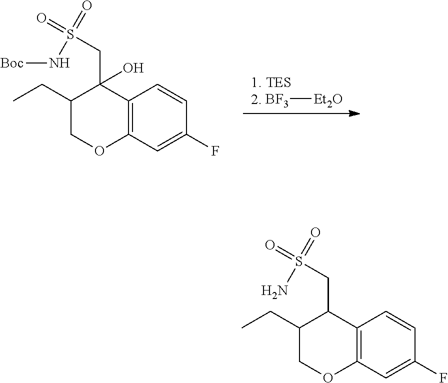

To a solution of tert-butyl (3-ethyl-7-fluoro-4-hydroxychroman-4-yl)methylsulfonylcarbamate (4.0 g, 10.24 mmol) in DCM (20 mL) was added TES (3.53 g, 30.5 mmol) at 0° C. After stirring for about 0.5 hours at this temperature, BF₃-Et₂O (6.2 g, 20.4 mmol) was added and the mixture was stirred at about 0° C. for an about additional 2 hours. Upon completion, the mixture was poured into sat. NaHCO₃ (80 mL), extracted with DCM (100 mL×2), and the combined organic layers were dried and concentrated. The resulting crude product was purified by silica gel column eluted with PE/EtOAc (2/1) to PE/EtOAc (1:1) to give 3-ethyl-7-fluorochroman-4-yl)methanesulfonamide as a diastereomeric mixture. MS (ESI): m/z 294 [M+Na]⁺.

### Synthesis of ((3S*,4R*)-3-ethyl-7-fluorochroman-4-yl)methanesulfonamide (Compound 20), ((3R*,4S*)-3-ethyl-7-fluorochroman-4-yl)methanesulfonamide (Compound 19), ((3S*,4S*)-3-ethyl-7-fluorochroman-4-yl)methanesulfonamide (Compound 22) and ((3R*, 4R*)-3-ethyl-7-fluorochroman-4-yl)methanesulfonamide (Compound 21)

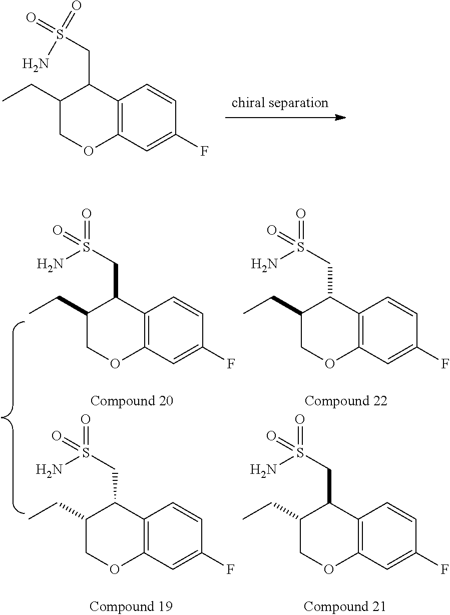

The diastereomeric mixture from Step 2 was separated using the chiral separation conditions below to yield Compound 20 (P1, retention time: 1.93 min), a mixture (P2) of Compound 19 and Compound 21, and Compound 22 (P3, retention time: 4.30 min).

Compound 20 (P1, retention time: 1.93 min). ¹H NMR (400 MHz, DMSO-d6) δ 7.24 (t, J=8.0 Hz, 1H), 7.02 (brs, 2H), 6.78-6.73 (m, 1H), 6.63-6.60 (m, 1H), 4.12 (s, 2H), 3.45-3.35 (m, 1H), 3.17-3.07 (m, 2H), 2.10-2.07 (m, 1H), 1.33-1.25 (m, 2H), 0.94 (t, J=7.2 Hz, 3H); and Compound 22 (P3, retention time: 4.30 min). ¹H NMR (400 MHz, DMSO-d6) δ 7.34 (s, 1H), 6.99 (brs, 2H), 6.67-6.58 (m, 2H), 4.16-4.13 (m, 1H), 4.00-3.97 (m, 1H), 3.41 (s, 1H), 3.23 (m, 2H), 2.00 (s, 1H), 1.43 (s, 1H), 1.24 (bs, 1H), 0.98 (s, 3H).

### Chiral HPLC Conditions:

| | |
|---|---|
| Instrument: SFC-150 (Thar, Waters) | Column: AS 20*250 mm, 10 um |
| | (Daicel) |
| Column temperature: 35° C. | Mobile phase: CO2/IPA(0.5% |
| | MeOH Ammonia) = 55/45 |
| Flow rate: 100 g/min | Back pressure: 100 bar |
| Detection wavelength: 214 nm | Cycle time: 5 min |
| Sample solution: 1000 mg | |
| dissolved in 40 ml MeOH | |
| Injection volume: 1.9 mL | |

The diastereomeric mixture, P2 from the first chiral separation described above (P2) of Compound 19 and Compound 21, was separated using a different chiral HPLC condition to provide the two diastereomers.

Compound 19: (P1, retention time: 0.69 min). ¹H NMR (400 MHz, DMSO-d6) δ 7.24 (t, J=7.6 Hz, 1H), 7.01 (brs, 2H), 6.77-6.72 (m, 1H), 6.63-6.60 (m, 1H), 4.12 (bs, 2H), 3.44-3.37 (m, 1H), 3.17-3.07 (m, 2H), 2.10-2.07 (m, 1H), 1.33-1.25 (m, 2H), 0.94 (t, J=7.2 Hz, 3H); and Compound 21: (P2, retention time: 1.11 min). ¹H NMR (400 MHz, DMSO-d6) δ 7.34 (t, J=7.6 Hz, 1H), 6.99 (brs, 2H), 6.67 (t, J=7.6 Hz, 1H), 6.60 (d, J=10.4 Hz, 1H), 4.14 (d, J=10.8 Hz, 1H), 3.97 (t, J=9.2 Hz, 1H), 3.41 (s, 1H), 3.27-3.17 (m, 2H), 2.00 (s, 1H), 1.47-1.40 (m, 1H), 1.25-1.18 (m, 1H), 0.97 (t, J=7.2 Hz, 3H).

### Chiral HPLC Conditions:

| | |
|---|---|
| Instrument: SFC-80 (Thar, Waters) | Column: IG 20*250 mm, 10 um |
| | (Daicel) |
| Column temperature: 35° C. | Mobile phase: CO₂/MeOH(0.2% |
| | MeOH Ammonia) = 75/25 |
| Flow rate: 80 g/min | Back pressure: 100 bar |
| Detection wavelength: 214 nm | Cycle time: 3.5 min |
| Sample solution: 300 mg | |
| dissolved in 25 ml MeOH | |
| Injection volume: 1.0 mL | |

### Example 9. Preparation of (R*)-1-((S*)-7-fluorochroman-4-yl)propane-1-sulfonamide (Compound 23), (S*)-1-((R*)-7-fluorochroman-4-yl)propane-1-sulfonamide (Compound 24), (S*)-1-((S*)-7-fluorochroman-4-yl)propane-1-sulfonamide (Compound 25) and (R*)-1-((R*)-7-fluorochroman-4-yl)propane-1-sulfonamide (Compound 26)

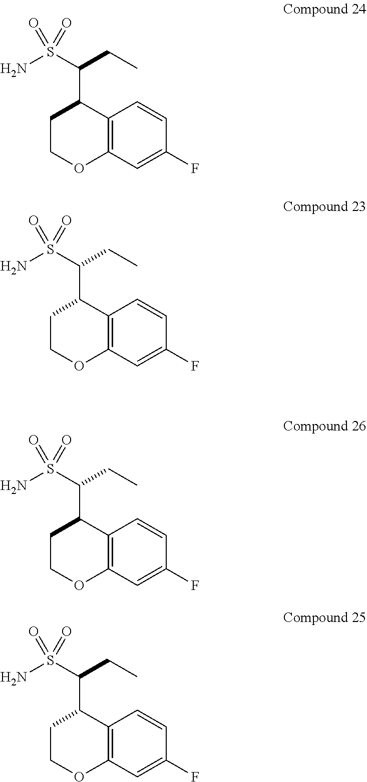 [SMILES: CC[C@@H]([C@@H]1CCOc2cc(F)ccc21)S(N)(=O)=O ; CC[C@H]([C@H]1CCOc2cc(F)ccc21)S(N)(=O)=O ; CC[C@H]([C@@H]1CCOc2cc(F)ccc21)S(N)(=O)=O ; CC[C@@H]([C@H]1CCOc2cc(F)ccc21)S(N)(=O)=O]

(R*)-1-((S*)-7-Fluorochroman-4-yl)propane-1-sulfonamide (Compound 23), (S*)-1-((R*)-7-fluorochroman-4-yl)propane-1-sulfonamide (Compound 24), (S*)-1-((S*)-7-fluorochroman-4-yl)propane-1-sulfonamide (Compound 25) and (R*)-1-((R*)-7-fluorochroman-4-yl)propane-1-sulfonamide (Compound 26) can be synthesized starting from 7-fluorochroman-4-one and tert-butyl (propylsulfonyl)carbamate using a similar procedure for the synthesis of compounds 47-50.

### Example 10. Preparation of (R*)-1-(7-fluorochroman-4-yl)cyclopropane-1-sulfonamide (Compound 28) and (S*)-1-(7-fluorochroman-4-yl)cyclopropane-1-sulfonamide (Compound 27)

### Synthesis of tert-butyl ((1-(7-fluoro-4-hydroxychroman-4-yl)cyclopropyl)sulfonyl)carbamate

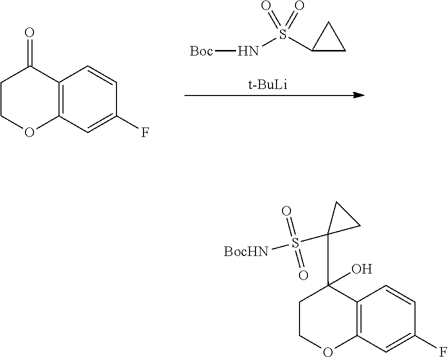 [SMILES: "t-BuLi" ; O=C1CCOc2cc(F)ccc21 ; CC(C)(C)OC(=O)NS(=O)(=O)C1CC1 ; CC(C)(C)OC(=O)NS(=O)(=O)C1(C2(O)CCOc3cc(F)ccc32)CC1]

To a solution of tert-butyl cyclopropylsulfonylcarbamate (6.92 g, 28.2 mmol) in THF (100 mL) was added t-BuLi (32.5 mL, 1.3 M in pentane, 42.3 mmol) at about −78° C., followed by a solution of 7-fluorochroman-4-one (2.4 g, 14.1 mmol) in THF (50 mL). The reaction was stirred at this temperature for about 2 hours and then at about room temperature for an about additional 2 hours. Saturated aqueous NH₄Cl (20 mL) was added to the reaction vessel and the resulting biphasic mixture was transferred to a separatory funnel. The layers were separated, and the aqueous layer was washed with EtOAc (3×40 mL) and water (2×20 mL). The combined organic layer was dried and concentrated in vacuo. The resulting substance was purified by silica gel column chromatography with a gradient elution of DCM/MeOH (100%:0%) to DCM/MeOH (90%:10%) to provide tert-butyl tert-butyl ((1-(7-fluoro-4-hydroxychroman-4-yl)cyclopropyl)sulfonyl)carbamate. MS (ESI): m/z=410[M+Na]⁺.

### Synthesis of 1-(7-fluorochroman-4-yl)cyclopropane-1-sulfonamide

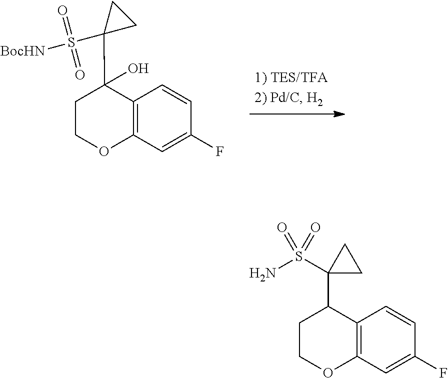 [SMILES: CC(C)(C)OC(=O)NS(=O)(=O)C1(C2(O)CCOc3cc(F)ccc32)CC1 ; NS(=O)(=O)C1(C2CCOc3cc(F)ccc32)CC1]

To a solution of tert-butyl tert-butyl ((1-(7-fluoro-4-hydroxychroman-4-yl)cyclopropyl)sulfonyl)carbamate (2.4 g, 4.95 mmol) in DCM (3 mL) was added TES (1.72 g, 14.8 mmol) and TFA (5.64 g, 49.5 mmol). The reaction was stirred at about room temperature for about 16 hours and then solvent removed. The residue was dissolved in MeOH (15 mL) and Pd/C (10%, wet) (728 mg) was added. The mixture was stirred at about room temperature under H₂ for about 2 hours. After filtration, the filtrate was concentrated and the residue was purified by silica gel column chromatography with a gradient elution of DCM/MeOH (100%:0%) to DCM/MeOH (50%:50%) to provide 1-(7-fluorochroman-4-yl)cyclopropane-1-sulfonamide. MS (ESI): m/z=294.1. [M+Na]⁺.

### Synthesis of (R*)-1-(7-fluorochroman-4-yl)cyclopropane-1-sulfonamide (Compound 28) and (S*)-1-(7-fluorochroman-4-yl)cyclopropane-1-sulfonamide (Compound 27)

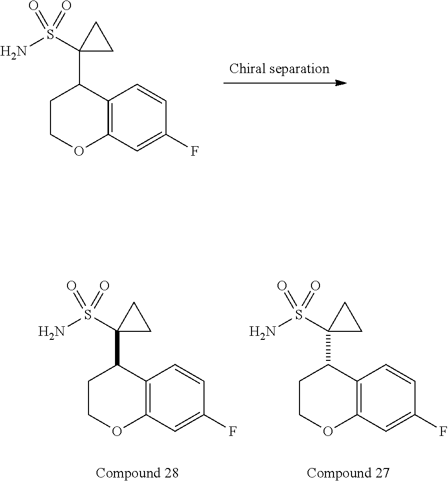 [SMILES: NS(=O)(=O)C1(C2CCOc3cc(F)ccc32)CC1 ; NS(=O)(=O)C1([C@@H]2CCOc3cc(F)ccc32)CC1 ; NS(=O)(=O)C1([C@H]2CCOc3cc(F)ccc32)CC1]

The racemic mixture of 1-(7-fluorochroman-4-yl)cyclopropane-1-sulfonamide (1.6 g) was separated by chiral HPLC under the following conditions to give the two enantiomers.

Compound 28 (P1) (retention time: 1.17 min). MS (ESI): m/z=294.1[M+Na]⁺. ¹H NMR (400 MHz, CD₃OD) δ 7.23-7.04 (m, 1H), 6.50 (td, J=8.5, 2.6 Hz, 1H), 6.38 (dd, J=10.4, 2.6 Hz, 1H), 4.30-4.18 (m, 1H), 4.13-4.05 (m, 1H), 3.60 (t, J=5.5 Hz, 1H), 2.48 (dtd, J=14.4, 5.7, 3.3 Hz, 1H), 2.06-1.88 (m, 1H), 1.40-1.25 (m, 1H), 1.22-1.12 (m, 1H), 0.88-0.77 (m, 1H), 0.59-0.45 (m, 1H); and Compound 27 (P2) (retention time: 2.17 min). MS (ESI): m/z=294.1[M+Na]⁺. ¹H NMR (400 MHz, CD₃OD) δ 7.13 (dd, J=8.4, 6.8 Hz, 1H), 6.50 (td, J=8.5, 2.6 Hz, 1H), 6.38 (dd, J=10.4, 2.6 Hz, 1H), 4.34-4.15 (m, 1H), 4.16-4.00 (m, 1H), 3.60 (t, J=5.5 Hz, 1H), 2.48 (dtd, J=14.5, 5.7, 3.3 Hz, 1H), 1.98 (ddd, J=18.8, 9.4, 4.6 Hz, 1H), 1.36-1.24 (m, 1H), 1.24-1.11 (m, 1H), 0.92-0.68 (m, 1H), 0.52 (ddd, J=9.7, 6.7, 5.3 Hz, 1H).

### Chiral HPLC Conditions:

| | |
|---|---|
| Instrument: SFC-80 (Thar, Waters) | Column: IG 20*250 mm, 10 um |
| | (Daicel) |
| Column temperature: 35° C. | Mobile phase: CO₂/MeOH (0.2% |
| | MeOH Ammonia) = 55/45 |
| Flow rate: 80 g/min | Back pressure: 100 bar |
| Detection wavelength: 214 nm | Cycle time: 4.4 min |
| Sample solution: 1600 mg | |
| dissolved in 60 ml MeOH | |
| Injection volume: 1.9 ml | |

### Example 11. Preparation of (R*)-(6,7-difluorochroman-4-yl)methanesulfonamide (Compound 30) and (S*)-(6,7-difluorochroman-4-yl)methanesulfonamide (Compound 29)

### Synthesis of 6,7-difluoro-4H-chromen-4-one

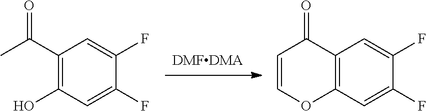

To 1-(4,5-difluoro-2-hydroxyphenyl)ethan-1-one (2 g, 11.0 mmol) was added DMF·DMA (20 mL) and the reaction was stirred at about 70° C. for about 2 hours under N₂. Water (100 mL) was added to the reaction vessel and the resulting biphasic mixture was transferred to a separatory funnel. The layers were separated, and the aqueous layer was extracted with EtOAc (3×200 mL). The combined organics were dried and concentrated in vacuo. The residue was dissolved in DCM (200 mL) and HCl (aq. 6 M, 20 mL) was added. The reaction was stirred at about 100° C. for about 2 hours. DCM (30 mL) was added to the reaction vessel and the resulting biphasic mixture was transferred to a separatory funnel. The layers were separated, and the aqueous layer was extracted with DCM (3×30 mL). The combined organics were dried and concentrated in vacuo. The resulting substance was purified by silica gel column chromatography with an isocratic elution of PE/EtOAc (50%:50%) to provide 6,7-difluoro-4H-chromen-4-one. MS (ESI): m/z=183.1 [M+H]⁺.

### Synthesis of 6,7-difluorochroman-4-one

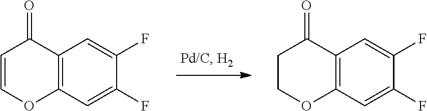 [SMILES: O=c1ccoc2cc(F)c(F)cc12 ; O=C1CCOc2cc(F)c(F)cc21]

To a solution of 6,7-difluoro-4H-chromen-4-one (12.4 g, 57.6 mmol) in EtOAc (120 mL) was added TEA (8.74 g, 86.4 mmol) and Pd/C (10%) (2.718 g, 4.14 mmol). The reaction was stirred at about room temperature for about 16 hours under Hz. After filtration, the filtrate was concentrated to provide 6,7-difluorochroman-4-one. MS (ESI): m/z=185.1 [M+H]⁺.

### Synthesis of tert-butyl (((6,7-difluoro-4-hydroxychroman-4-yl)methyl)-sulfonyl)carbamate

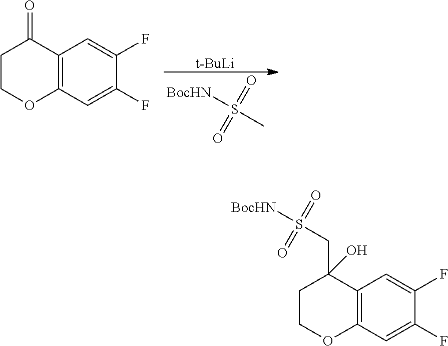 [SMILES: "t-BuLi" ; O=C1CCOc2cc(F)c(F)cc21 ; CC(C)(C)OC(=O)NS(C)(=O)=O ; CC(C)(C)OC(=O)NS(=O)(=O)CC1(O)CCOc2cc(F)c(F)cc21]

To a solution of tert-butyl methylsulfonylcarbamate (8.9 g, 45.6 mmol) in THF (150 mL) was added t-BuLi (1.3 M in pentane, 70.2 mL, 91.2 mmol). The mixture was stirred at about −78° C. for 1 h and 6,7-difluorochroman-4-one (6.6 g, 30.4 mmol) in THF (20 mL) was added. The reaction mixture was stirred at about room temperature for about 16 hours and saturated NH₄Cl (aq. 30 mL) was added to the reaction vessel. The resulting biphasic mixture was transferred to a separatory funnel. The layers were separated, and the aqueous phase was extracted with EtOAc (3×30 mL). The combined organics were dried and concentrated in vacuo. The resulting substance was purified by silica gel column chromatography with an isocratic elution of DCM/MeOH (80%:20%) to provide tert-butyl N-[(6,7-difluoro-4-hydroxy-3,4-dihydro-2H-1-benzopyran-4-yl)methanesulfonyl]carbamate. MS (ESI): m/z=401.8 [M+Na]⁺.

### Synthesis of (6,7-difluorochroman-4-yl)methanesulfonamide

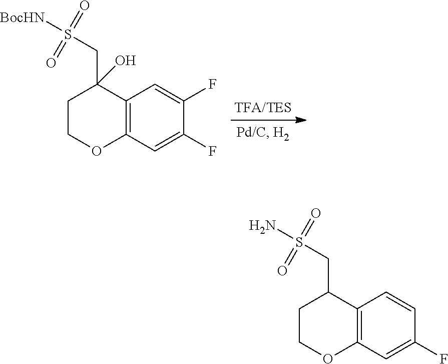 [SMILES: CC(C)(C)OC(=O)NS(=O)(=O)CC1(O)CCOc2cc(F)c(F)cc21 ; NS(=O)(=O)CC1CCOc2cc(F)ccc21]

To a solution of tert-butyl N-[(6,7-difluoro-4-hydroxy-3,4-dihydro-2H-1-benzo pyran-4-yl)methanesulfonyl]carbamate (3.6 g, 9.48 mmol) in DCM (40 mL) was added TES (3.30 g, 28.4 mmol) and TFA (10.8 g, 94.8 mmol). The reaction mixture was stirred at about room temperature for about 16 hours and concentrated in vacuo. The residue was dissolved in MeOH (50 mL) and Pd/C (10%) (460 mg) was added. The mixture was stirred at about room temperature under H₂ for about 2 hours. After filtration, the filtrate was concentrated to provide ((6,7-difluorochroman-4-yl)methanesulfonamide. MS (ESI): m/z=286.0 [M+Na]⁺.

### Synthesis of (R*)-(6,7-difluorochroman-4-yl)methanesulfonamide (Compound 30) and (S*)-(6,7-difluorochroman-4-yl)methanesulfonamide (Compound 29)

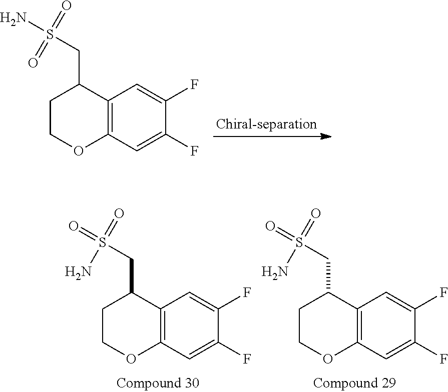

((6,7-Difluorochroman-4-yl)methanesulfonamide was separated by chiral HPLC under the following conditions to give the two enantiomers.

Compound 30 (P1) (retention time=0.927 min). MS (ESI): m/z=286.0. [M+Na]⁺. ¹H NMR (400 MHz, CD₃OD) δ 7.05 (dd, J=11.2, 9.0 Hz, 1H), 6.56 (dd, J=11.9, 7.1 Hz, 1H), 4.21-3.95 (m, 2H), 3.42-3.23 (m, 3H), 2.23-1.98 (m, 2H); and Compound 29 (P2) (retention time=1.202 min). MS (ESI): m/z=286.0. [M+Na]⁺. ¹H NMR (500 MHz, CD₃OD) δ 7.05 (dd, J=11.2, 8.9 Hz, 1H), 6.56 (dd, J=11.8, 7.1 Hz, 1H), 4.16-4.00 (m, 2H), 3.35-3.25 (m, 3H), 2.18-2.04 (m, 2H).

### Chiral HPLC Conditions:

| | |
|---|---|
| Instrument: SFC-80 (Thar, Waters) | Column: AY 20 × 250 mm, 10 um |
| | (Daicel) |
| Column temperature: 35° C. | Mobile phase: CO₂/EtOH (0.5% |
| | MeOH Ammonia) = 80/20 |
| Flow rate: 80 g/min | Back pressure: 100 bar |
| Detection wavelength: 214 nm | Cycle time: 5.5 min |
| Sample solution: 3500 mg | |
| dissolved in 90 mL MeOH | |
| Injection volume: 0.6 mL | |

### Example 12. Preparation of (R*)-(7-chlorochroman-4-yl)methanesulfonamide (Compound 32) and (S*)-(7-chlorochroman-4-yl)methanesulfonamide (Compound 31)

### Synthesis of tert-butyl (((7-chloro-4-hydroxychroman-4-yl) methyl)sulfonyl)-carbamate

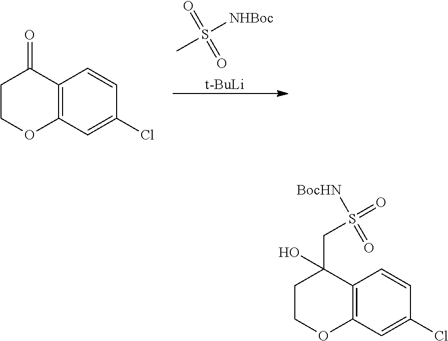 [SMILES: "t-BuLi" ; O=C1CCOc2cc(Cl)ccc21 ; CC(C)(C)OC(=O)NS(C)(=O)=O ; CC(C)(C)OC(=O)NS(=O)(=O)CC1(O)CCOc2cc(Cl)ccc21]

To a solution of tert-butyl N-methanesulfonylcarbamate (3.82 g, 19.6 mmol) in THF (30 mL) was added t-BuLi (1.3 M in heptane, 30.1 mL, 39.2 mmol) at about −78° C. After stirring at about −78° C. for 1 h, 7-chlorochroman-4-one (2 g, 10.9 mmol) in THF (10 mL) was added. The mixture was stirred at about −78° C. for about 2 hours. Upon completion, water (80 mL) was added to quenched the reaction and citric acid was added to adjust the pH to 5. The mixture was extracted with EtOAc (100 mL×2) and the combined organic layers were dried and concentrated. The resulting substance was purified by silica gel column chromatography with a gradient elution of PE/EtOAc (100%:0%) to PE/EtOAc (70%:30%) to provide tert-butyl (((7-chloro-4-hydroxychroman-4-yl)methyl)sulfonyl)carbamate. MS (ESI): m/z=400 [M+Na]⁺.

### Synthesis of tert-butyl (((7-chlorochroman-4-yl)methyl)sulfonyl)carbamate

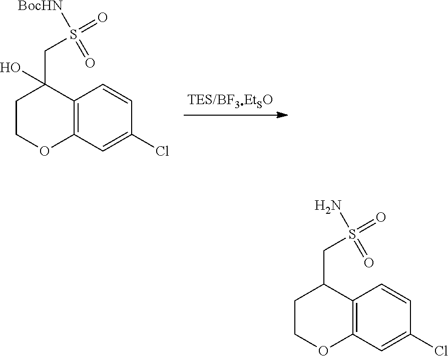 [SMILES: CC(C)(C)OC(=O)NS(=O)(=O)CC1(O)CCOc2cc(Cl)ccc21 ; NS(=O)(=O)CC1CCOc2cc(Cl)ccc21]

To a solution of crude tert-butyl (((7-chloro-4-hydroxychroman-4-yl)methyl)-sulfonyl)carbamate in DCM (40 mL) was added TES (3.22 g, 27.7 mmol) at about 0° C. After stirring at about 0° C. for about 30 min., BF₃·Et₂O (8.36 g, 58.9 mmol) was added. The reaction was stirred at about room temperature for about 3 hours. Upon completion, the mixture was filtered and the filter cake was recrystallizated from acetone and water to give tert-butyl (((7-chlorochroman-4-yl)methyl)sulfonyl)carbamate. MS (ESI): m/z=262 [M+H]⁺.

### Synthesis of (R*)-(7-chlorochroman-4-yl)methanesulfonamide (Compound 32) and (S*)-(7-chlorochroman-4-yl)methanesulfonamide (Compound 31)

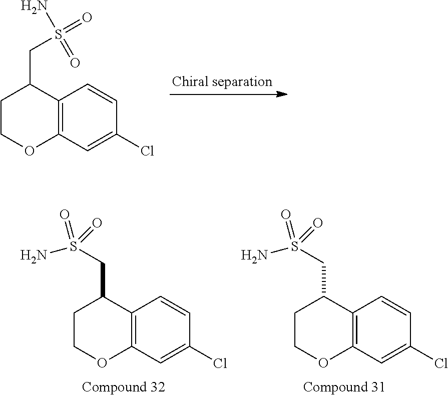 [SMILES: NS(=O)(=O)CC1CCOc2cc(Cl)ccc21 ; NS(=O)(=O)C[C@@H]1CCOc2cc(Cl)ccc21 ; NS(=O)(=O)C[C@H]1CCOc2cc(Cl)ccc21]

tert-Butyl (((7-chlorochroman-4-yl)methyl)sulfonyl)carbamate (674 mg) was separated by chiral HPLC using the following conditions to give the two enantiomers.

Compound 32 (P1) (retention time=0.94 min). ¹H NMR (400 MHz, CD₃OD) δ 7.20 (d, J=8.3 Hz, 1H), 6.89 (dd, J=8.3, 2.2 Hz, 1H), 6.80 (d, J=2.1 Hz, 1H), 4.25 (dt, J=11.1, 4.3 Hz, 1H), 4.22-4.14 (m, 1H), 3.51-3.35 (m, 3H), 2.31 (ddd, J=14.4, 7.3, 4.5 Hz, 1H), 2.22 (ddd, J=14.3, 9.4, 4.5 Hz, 1H); and Compound 31 (P2) (retention time=1.19 min). ¹H NMR (400 MHz, CD₃OD) δ 7.18 (d, J=8.3 Hz, 1H), 6.87 (dd, J=8.3, 2.2 Hz, 1H), 6.78 (d, J=2.1 Hz, 1H), 4.27-4.20 (m, 1H), 4.20-4.12 (m, 1H), 3.50-3.53 (m, 3H), 2.27 (dd, J=7.4, 4.6 Hz, 1H), 2.21 (dd, J=9.6, 4.5 Hz, 1H).

### Chiral HPLC Conditions:

| | |
|---|---|
| Instrument: SFC-80 (Thar, | Column: AY 20 × 250 mm, 10 um |
| Waters) | (Daicel) |
| Column temperature: 35° C. | |
| Mobile phase: CO2/MeOH (0.2% | |
| MeOH Ammonia) = 75/25 | |
| Flow rate: 80 g/min | Back pressure: 100 bar |
| Detection wavelength: 214 nm | Cycle time: 3.1 min |
| Sample solution: 670 mg | |
| dissolved in 35 ml MeOH | |
| Injection volume: 1.0 mL | |

### Example 13. Preparation of ((2S*,4R*)-2-methylchroman-4-yl)methanesulfonamide (Compound 34), ((2R*,4S*)-2-methylchroman-4-yl)methanesulfonamide (Compound 33), ((2S*,4S*)-2-methylchroman-4-yl)methane-sulfonamide (Compound 35) and ((2R*,4R*)-2-methylchroman-4-yl)methanesulfonamide (Compound 36)

### Synthesis of tert-butyl (((4-hydroxy-2-methylchroman-4-yl)methyl)sulfonyl)carbamate

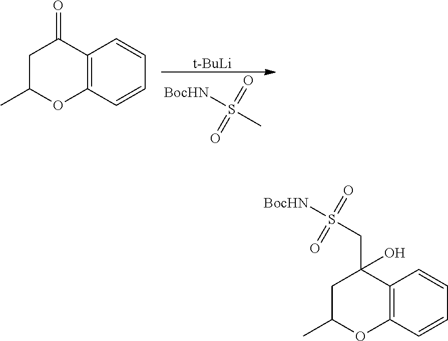 [SMILES: "t-BuLi" ; CC1CC(=O)c2ccccc2O1 ; CC(C)(C)OC(=O)NS(C)(=O)=O ; CC1CC(O)(CS(=O)(=O)NC(=O)OC(C)(C)C)c2ccccc2O1]

To a solution of tert-butyl (methylsulfonyl)carbamate (3.12 g, 16.0 mmol) in THF (40 mL) was added t-BuLi (1.3 N in hexane, 24.6 mL, 32.0 mmol) at about −78° C. under N₂. After stirring for about 30 min., a solution of 2-methylchroman-4-one (1.85 g, 10.7 mmol) in THF (40 mL) was added. The reaction was stirred and warmed gradually to room temperature over about 2 hours. Saturated NH₄Cl (aq. 100 mL) was added to the reaction vessel. The mixture was acidified with saturated critic acid (aq.) till pH around 4 and extracted with EtOAc (3×100 mL). The combined organics were dried and concentrated in vacuo. The resulting substance was purified by flash column chromatography eluted with DCM/MeOH (95%:5%) to provide tert-butyl (((4-hydroxy-2-methylchroman-4-yl)methyl)sulfonyl)carbamate. MS (ESI): m/z=380.1. [M+Na]⁺.

### Synthesis of (2-methylchroman-4-yl)methanesulfonamide

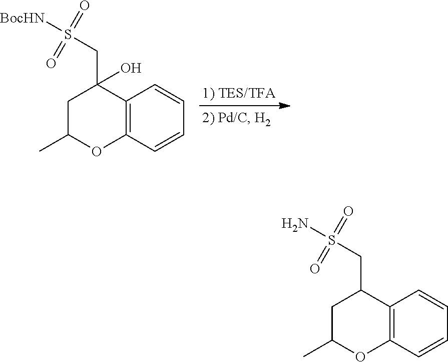 [SMILES: CC1CC(O)(CS(=O)(=O)NC(=O)OC(C)(C)C)c2ccccc2O1 ; CC1CC(CS(N)(=O)=O)c2ccccc2O1]

To a solution of tert-butyl (((4-hydroxy-2-methylchroman-4-yl)methyl)sulfonyl)carbamate (1.29 g, 3.10 mmol) in DCM (10 mL) was added TFA (2.82 g, 24.8 mmol) and TES (1.08 g, 9.30 mmol). After stirring at about room temperature for about 16 hours, the mixture was concentrated. The residue was dissolved in MeOH (15 mL) and Pd/C (10% wet, 567 mg) was added. The mixture was stirred at about ambient temperature under Hz for about 2 hours. After filtration, the solvent was removed in vacuo to afford crude (2-methylchroman-4-yl)methanesulfonamide as a mixture of 4 diastereomers, with a ratio of 20:1 for the two pair of enantiomers based on LCMS analysis.

### Synthesis of ((2S*,4R*)-2-methylchroman-4-yl)methanesulfonamide (Compound 34), ((2R*,4S*)-2-methylchroman-4 yl)methanesulfonamide (Compound 33), ((2S*,4S*)-2-methylchroman-4-yl)methane-sulfonamide (Compound 35) and ((2R*,4R*)-2-methylchroman-4-yl)methanesulfonamide (Compound 36)

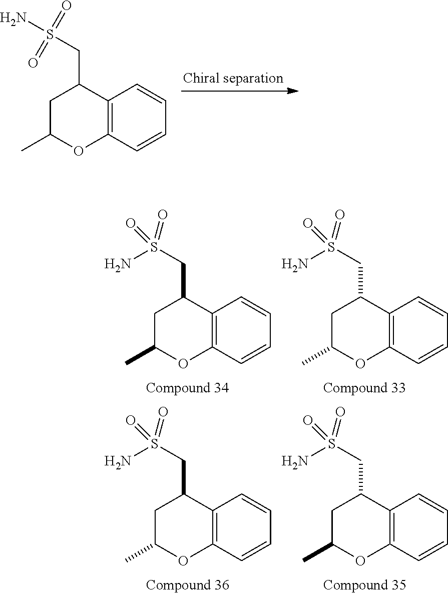

The diastereomeric mixture from step 2 was separated by chiral HPLC to afford the two major enantiomers.

Compound 34 (P1) (retention time: 2.49 min). MS(ESI): m/z=264.1[M+Na]⁺. ¹H NMR (400 MHz, CD₃OD) δ 7.30-7.28 (d, J=7.8 Hz, 1H), 7.12-7.08 (t, J=7.7 Hz, 1H), 6.91-6.88 (t, J=7.4 Hz, 1H), 6.78-6.76 (dd, J=8.1 Hz, 1H), 4.13-4.09 (m, 1H), 4.09-4.08 (dd, J=14.3, 2.8 Hz, 1H), 3.83-3.79 (dd, J=17.7, 7.2 Hz, 1H), 3.57-3.53 (dd, J=14.3, 9.7 Hz, 1H), 3.33-3.32 (dd, J=13.6, 6.4 Hz, 1H), 2.63-2.58 (dt, J=13.6, 11.3 Hz, 1H), 1.65-1.56 (d, J=6.2 Hz, 1H), 1.41-1.39 (d, 3H); and Compound 33 (P2) (retention time: 2.92 min). MS(ESI): m/z=264.1[M+Na]⁺. ¹H NMR (400 MHz, CD₃OD) δ 7.30-7.28 (d, J=7.8 Hz, 1H), 7.12-7.08 (t, J=7.7 Hz, 1H), 6.91-6.88 (t, J=7.4 Hz, 1H), 6.78-6.76 (dd, J=8.1 Hz, 1H), 4.13-4.09 (m, 1H), 4.09-4.08 (dd, J=14.3, 2.8 Hz, 1H), 3.83-3.79 (dd, J=17.7, 7.2 Hz, 1H), 3.57-3.53 (dd, J=14.3, 9.7 Hz, 1H), 3.33-3.32 (dd, J=13.6, 6.4 Hz, 1H), 2.63-2.58 (dt, J=13.6, 11.3 Hz, 1H), 1.65-1.56 (d, J=6.2 Hz, 1H), 1.41-1.39 (d, 3H).

### Chiral HPLC Conditions:

| | |
|---|---|
| Instrument: SFC-80 (Thar, | Column: OJ 20*250 mm, 10 um |
| Waters) | (Daicel) |
| Column temperature: 35° C. | Mobile phase: CO₂/MeOH(0.2% |
| | MeOH Ammonia) = 85/15 |
| Flow rate: 80 g/min | Back pressure: 100 bar |
| Detection wavelength: 214 nm | Cycle time: 6.8 min |
| Sample solution: 600 mg | |
| dissolved in 35 ml MeOH | |
| Injection volume: 1.0 mL | |

### Example 14. Preparation of (R*)-(7-(trifluoromethyl)chroman-4-yl)methanesulfonamide (Compound 38) and (S*)-(7-(trifluoromethyl)chroman-4-yl)methanesulfonamide (Compound 37)

### Synthesis of tert-butyl (4-hydroxy-7-(trifluoromethyl)chroman-4-yl)methylsulfonylcarbamate

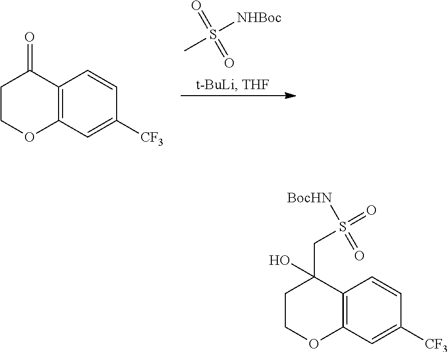

To a solution of tert-butyl N-methanesulfonylcarbamate (1.17 g, 6.00 mmol) in THF (20 mL) was added t-BuLi (9.23 mL, 1.3 M in pentane, 12.0 mmol) at about −78° C. The reaction was stirred at that temperature for about 1 hour and 7-(trifluoromethyl)chroman-4-one (1 g, 4.62 mmol) was added. The reaction was then stirred at about room temperature for about 16 hours. Water (50 mL) was added to the reaction vessel and the resulting biphasic mixture was transferred to a separatory funnel. The organic layers were separated and washed with saturated aqueous NaCl (2×50 mL). The combined organics were dried and concentrated in vacuo to provide tert-butyl (4-hydroxy-7-(trifluoromethyl)chroman-4yl)methylsulfonylcarbamate. MS(ESI): m/z 434 [M+Na]⁺.

### Synthesis of (7-(trifluoromethyl)chroman-4-yl)methanesulfonamide

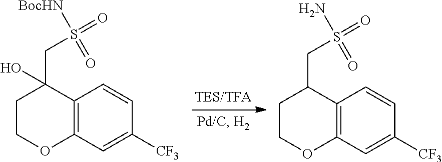 [SMILES: CC(C)(C)OC(=O)NS(=O)(=O)CC1(O)CCOc2cc(C(F)(F)F)ccc21 ; NS(=O)(=O)CC1CCOc2cc(C(F)(F)F)ccc21]

To a solution of tert-butyl (4-hydroxy-7-(trifluoromethyl)chroman-4-yl)methylsulfonylcarbamate (1.5 g, 3.37 mmol) in DCM (80 mL) was added TFA (3.06 g, 26.9 mmol) and TES (1.17 g, 10.1 mmol). The reaction was stirred at about room temperature for about 16 hours and the solvent removed in vacuo. The residue was dissolved in EtOAc (50 mL) and Pd/C (0.7 g, 0.6568 mmol) was added. The reaction was stirred under H₂ at about room temperature for about 16 hours. After filtration, the filtrate was purified by silica gel column chromatography with a gradient elution of DCM/MeOH (100%:0%) to DCM/MeOH (90%:10%) to provide (7-(trifluoromethyl)chroman-4-yl)methanesulfonamide. MS(ESI): m/z 318 [M+23]⁺.

### Synthesis of (R*)-(7-(trifluoromethyl)chroman-4-yl)methanesulfonamide (Compound 38) and (S*)-(7-(trifluoromethyl)chroman-4-yl)methanesulfonamide (Compound 37)

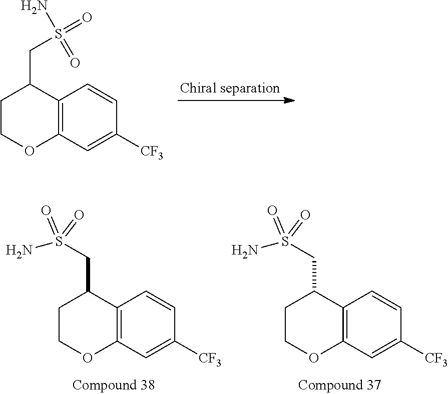 [SMILES: NS(=O)(=O)CC1CCOc2cc(C(F)(F)F)ccc21 ; NS(=O)(=O)C[C@@H]1CCOc2cc(C(F)(F)F)ccc21 ; NS(=O)(=O)C[C@H]1CCOc2cc(C(F)(F)F)ccc21]

The racemic mixture of (7-(trifluoromethyl)chroman-4-yl)methanesulfonamide was separated by chiral HPLC under the following conditions to give the two enantiomers.

Compound 38 (P1) (retention time: 0.842 min). MS(ESI): m/z 318 [M+Na]⁺. ¹H NMR (400 MHz, CD₃OD) δ 7.43 (d, J=8.0 Hz, 1H), 7.17 (d, J=7.2 Hz, 1H), 7.05 (s, 1H), 4.43-4.17 (m, 2H), 3.63-3.52 (m, 1H), 3.51-3.37 (m, 2H), 2.45-2.21 (m, 2H); and Compound 37 (P2) (retention time: 1.027 min). MS(ESI): m/z 318 [M+23]⁺. ¹H NMR (400 MHz, MeOD) δ 7.43 (d, J=8.0 Hz, 1H), 7.17 (d, J=8.2 Hz, 1H), 7.05 (s, 1H), 4.40-4.16 (m, 2H), 3.61-3.52 (m, 1H), 3.52-3.36 (m, 2H), 2.46-2.19 (m, 2H).

### Chiral HPLC Conditions:

| | |
|---|---|
| Instrument: SFC-150 (Thar, Waters) | Column: IG 20*250 mm, 10 um |
| | (Daicel) |
| Column temperature: 35° C. | Mobile phase: CO2/MeOH(0.2% |
| | MeOH Ammonia) = 70/30 |
| Flow rate: 100 g/min | Back pressure: 100 bar |
| Detection wavelength: 214 nm | Cycle time: 3 min |
| Sample solution: 850 mg | |
| dissolved in 30 ml MeOH | |
| Injection volume: 1.9 mL | |

### Example 15. Preparation of ((3S*,4R*)-3-methylchroman-4-yl)methanesulfonamide (Compound 40), ((3R*,4S*)-3-methylchroman-4-yl)methanesulfonamide (Compound 39), ((3S*,4S*)-3-methylchroman-4-yl)methanesulfonamide (Compound 41) and ((3R*,4R*)-3-methylchroman-4-yl)methanesulfonamide (Compound 42)

### Synthesis of tert-butyl (4-hydroxy-3-methylchroman-4-yl)methyl-sulfonylcarbamate

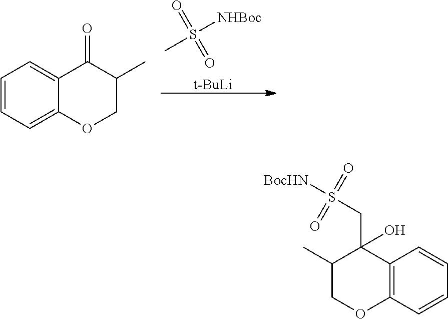

To a solution of tert-butyl N-methanesulfonylcarbamate (5.44 g, 27.9 mmol) in THF (200 mL) was added t-BuLi (43 mL, 1.3 M in pentane, 55.9 mmol) at about −78° C. The reaction was stirred at this temperature for about 0.5 hours and a solution of 3-methylchroman-4-one (3.6 g, 21.5 mmol) in 10 mL THF was added dropwise. The reaction was stirred at about room temperature for about 16 hours. Water (100 mL) and EtOAc (200 mL) were added to the reaction vessel and the resulting biphasic mixture was transferred to a separatory funnel. The organic layers were separated and washed with saturated aqueous NaCl (2×50 mL). The combined organics were dried and concentrated in vacuo. The residue was purified by silica gel column chromatography with a gradient elution of PE/EtOAc (100%:0%) to PE/EtOAc (80%:20%) to provide tert-butyl (4-hydroxy-3-methylchroman-4-yl)methylsulfonylcarbamate. MS(ESI): m/z 380 [M+Na]⁺

### Synthesis of (3-methylchroman-4-yl)methanesulfonamide

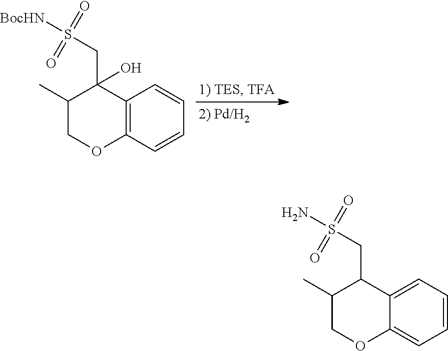 [SMILES: CC1COc2ccccc2C1(O)CS(=O)(=O)NC(=O)OC(C)(C)C ; CC1COc2ccccc2C1CS(N)(=O)=O]

To a solution of tert-butyl (4-hydroxy-3-methylchroman-4-yl)methylsulfonylcarbamate (7.1 g, 17.8 mmol) in DCM (80 mL) was added TES (6.20 g, 53.4 mmol) and TFA (16.1 g, 142 mmol). The reaction was stirred at about room temperature for about 16 hours and then solvent removed in vacuo. The residue was dissolved in EtOAc (100 mL) and to it was added Pd/C (7 g, 6.57 mmol). The reaction was stirred under H₂ at about room temperature for about 16 hours. After filtration, the filtrate was concentrated in vacuo to afford (3-methylchroman-4-yl)methanesulfonamide (4.27 g) as a mixture of diastereomers. MS (ESI): m/z 264 [M+1]⁺.

### Synthesis of ((3S*,4R*)-3-methylchroman-4-yl)methanesulfonamide (Compound 40), ((3R*,4S*)-3-methylchroman-4-yl)methanesulfonamide (Compound 39), ((3S*,4S*)-3-methylchroman-4-yl)methanesulfonamide (Compound 41) and ((3R*,4R*)-3-methylchroman-4-yl)methanesulfonamide (Compound 42)

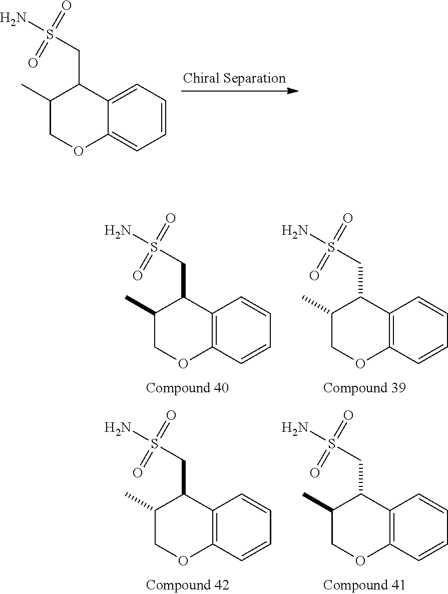

The diastereomeric mixture of (3-methylchroman-4-yl)methanesulfonamide was separated by chiral HPLC under the following conditions to give the four diastereomers.

Compound 40 (P1) (retention time: 2.697 min). MS(ESI): m/z 264 [M+1]⁺. ¹H NMR (400 MHz, CD₃OD) δ 7.26-7.20 (m, 1H), 7.15-7.08 (m, 1H), 6.93-6.87 (m, 1H), 6.80-6.74 (m, 1H), 4.19-4.13 (m, 1H), 4.04-3.96 (m, 1H), 3.44 (dd, J=14.6, 9.3 Hz, 1H), 3.39-3.34 (m, 1H), 3.11 (d, J=9.2 Hz, 1H), 2.54-2.40 (m, 1H), 1.10 (d, J=7.1 Hz, 3H); Compound 39 (P2) (retention time: 3.730 min). MS(ESI): m/z 264 [M+1]⁺. ¹H NMR (400 MHz, CD₃OD) δ 7.26-7.20 (m, 1H), 7.15-7.08 (m, 1H), 6.94-6.86 (m, 1H), 6.80-6.74 (m, 1H), 4.21-4.13 (m, 1H), 4.04-3.97 (m, 1H), 3.50-3.40 (m, 1H), 3.40-3.34 (m, 1H), 3.11 (d, J=9.4 Hz, 1H), 2.54-2.42 (m, 1H), 1.10 (d, J=7.1 Hz, 3H); Compound 42 (P3) (retention time: 4.270 min). MS(ESI): m/z 264 [M+1]⁺. ¹H NMR (400 MHz, CD₃OD) δ 7.33 (d, J=7.7 Hz, 1H), 7.16-7.07 (m, 1H), 6.92-6.82 (m, 1H), 6.79-6.72 (m, 1H), 4.21-4.11 (m, 1H), 4.02-3.91 (m, 1H), 3.61-3.53 (m, 1H), 3.53-3.37 (m, 2H), 2.53-2.38 (m, 1H), 1.09 (d, J=7.0 Hz, 3H); and Compound 41 (P4) (retention time: 5.137 min). MS(ESI): m/z 264 [M+1]⁺. ¹H NMR (400 MHz, CD₃OD) δ 7.33 (d, J=7.7 Hz, 1H), 7.11 (t, J=7.1 Hz, 1H), 6.87 (t, J=7.5 Hz, 1H), 6.75 (d, J=8.1 Hz, 1H), 4.23-4.10 (m, 1H), 4.03-3.91 (m, 1H), 3.59-3.52 (m, 1H), 3.52-3.36 (m, 2H), 2.52-2.38 (m, 1H), 1.09 (d, J=7.0 Hz, 3H).

### Chiral HPLC Conditions:

| | |
|---|---|
| Instrument: SFC-200 (Thar, Waters) | Column: IG 20*250 mm, 10 um |
| | (Daicel) |
| Column temperature: 35° C. | Mobile phase: CO₂/MeOH(0.2% |
| | MeOH Ammonia) = 90/10 |
| Flow rate: 140 g/min | Back pressure: 100 bar |
| Detection wavelength: 214 nm | Cycle time: 9.5 min |
| Sample solution: 4600 mg dissolved | |
| in 45 ml MeOH | |
| Injection volume: 0.8 mL | |

### Example 16. Preparation of (R*)-(7-fluoro-3,3-dimethylchroman-4-yl)methanesulfonamide (Compound 44) and (S*)-(7-fluoro-3,3-dimethylchroman-4-yl)methanesulfonamide (Compound 43)

### Synthesis of 3-(2-bromo-5-fluorophenoxy)-2,2-dimethylpropan-1-ol

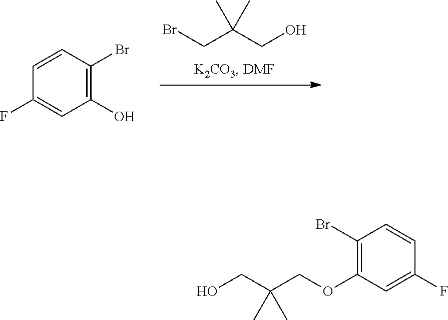

To a solution of 2-bromo-5-fluorophenol (3 g, 15.7 mmol) in DMF (50 mL) was added K₂CO₃ (4.32 g, 31.3 mmol), 3-bromo-2,2-dimethylpropan-1-ol (2.60 g, 15.6 mmol). The reaction mixture was stirred at about 120° C. for about 16 hours. EtOAc (100 mL) and water (200 mL) was added to the reaction vessel and the resulting biphasic mixture was transferred to a separatory funnel. The layers were separated, and the organic phase was washed with water (2×50 mL) and saturated aqueous NaCl (2×50 mL). The combined organics were dried and concentrated in vacuo. The resulting substance was purified by silica gel column chromatography with a gradient elution of PE/EtOAc (100%:0%) to PE/EtOAc (70%:30%) to provide 3-(2-bromo-5-fluorophenoxy)-2,2-dimethylpropan-1-ol (1.20 g). MS (ESI): m/z=259, 261 [M-OH]⁺.

### Synthesis of 3-(2-bromo-5-fluorophenoxy)-2,2-dimethylpropanal

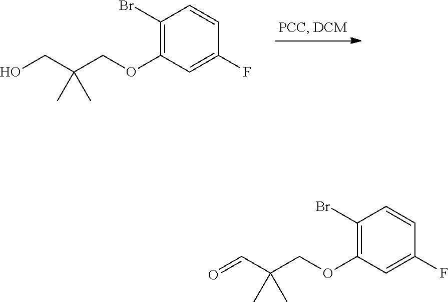

To a solution of 3-(2-bromo-5-fluorophenoxy)-2,2-dimethylpropan-1-ol (1.2 g, 4.33 mmol) in DCM (50 mL) was added PCC (1.21 g, 5.62 mmol). The reaction mixture was stirred at about room temperature for about 4 hours and solvent removed. The resulting substance was purified by silica gel column chromatography with a gradient elution of PE/EtOAc (100%:0%) to PE/EtOAc (90%:10%) to provide 3-(2-bromo-5-fluorophenoxy)-2,2-dimethylpropanal. MS (ESI): m/z=257, 259 [M-OH]⁺.

### Synthesis of 7-fluoro-3,3-dimethylchroman-4-one

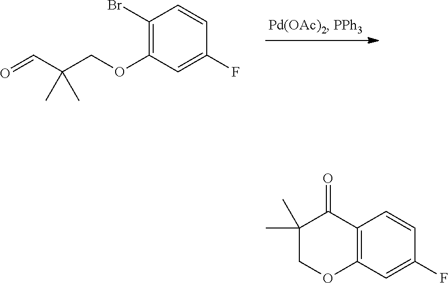

To a solution of 3-(2-bromo-5-fluorophenoxy)-2,2-dimethylpropanal (1 g, 3.63 mmol) in 1,4-dioxane (20 mL) was added Pd(OAc)₂ (81.4 mg, 363 μmol), PPh₃ (237 mg, 907 μmol) and K₂CO₃ (1.49 g, 10.8 mmol) under N₂. The reaction mixture was heated to about 120° C. and stirred at this temperature for about 16 hours. Water (40 mL) and EtOAc (40 mL) were added to the reaction vessel and the resulting biphasic mixture was transferred to a separatory funnel. The layers were separated, and the organic phase was washed with water (2×40 mL) and saturated aqueous NaCl (2×40 mL). The combined organics were dried and concentrated in vacuo. The resulting substance was purified by silica gel column chromatography with a gradient elution of PE/EtOAc (100%:0%) to PE/EtOAc (70%:30%) to provide 7-fluoro-3,3-dimethylchroman-4-one. MS (ESI): m/z=195 [M+H]⁺.

### Synthesis of tert-butyl (((7-fluoro-4-hydroxy-3,3-dimethylchroman-4-yl)methyl)sulfonyl)carbamate

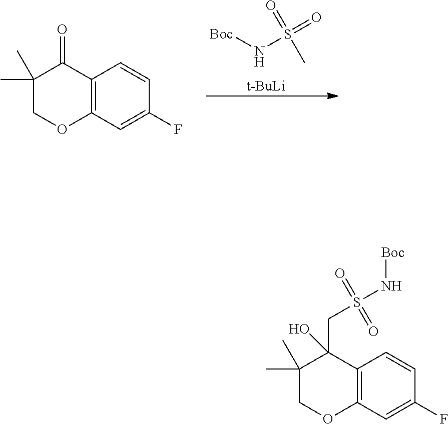 [SMILES: "t-BuLi" ; CC1(C)COc2cc(F)ccc2C1=O ; CC(C)(C)OC(=O)NS(C)(=O)=O ; CC(C)(C)OC(=O)NS(=O)(=O)CC1(O)c2ccc(F)cc2OCC1(C)C]

To a solution of tert-butyl (methylsulfonyl)carbamate (570 mg, 2.92 mmol) in THF (30 mL) was added t-BuLi (1.3 M in pentane, 4.5 mL, 5.84 mmol) at about −78° C. under N₂. The mixture was stirred at this temperature for about 30 min., and 7-fluoro-3,3-dimethyl-3,4-dihydro-2H-1-benzopyran-4-one (380 mg, 1.95 mmol) in THF (30 mL) was then added. The reaction was stirred and gradually warmed to room temperature over a period of about 2 hours. Saturated aqueous NH₄Cl (20 mL) and EtOAc (20 mL) were added to the reaction vessel and the resulting biphasic mixture was transferred to a separatory funnel. The layers were separated, and the organic phase was washed with water (50 mL) and saturated aqueous NaCl (50 mL). The combined organics were dried and concentrated in vacuo. The resulting substance was purified by silica gel column chromatography with a gradient elution of PE/EtOAc (100%:0%) to PE/EtOAc (50%:50%) to provide tert-butyl N-[(7-fluoro-4-hydroxy-3,3-dimethyl-3,4-dihydro-2H-1-benzopyran-4-yl)methanesulfonyl]carbamate. MS (ESI): m/z=412 [M+Na]⁺

### Synthesis of (7-fluoro-3,3-dimethylchroman-4-yl)methanesulfonamide

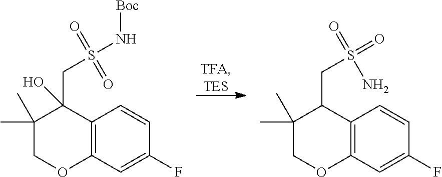 [SMILES: CC(C)(C)OC(=O)NS(=O)(=O)CC1(O)c2ccc(F)cc2OCC1(C)C ; "TFA,TES" ; CC1(C)COc2cc(F)ccc2C1CS(N)(=O)=O]

To a solution of tert-butyl N-[(7-fluoro-4-hydroxy-3,3-dimethyl-3,4-dihydro-2H-1-benzopyran-4-yl)methanesulfonyl]carbamate (510 mg, 1.30 mmol) in DCM (10 mL) was added TFA (4.82 mL, 65.0 mmol) and TES (2.06 mL, 13.0 mmol). The reaction mixture was heated to reflux and stirred at this temperature for about 4 hours. The mixture was concentrated to give (7-fluoro-3,3-dimethyl-3,4-dihydro-2H-1-benzopyran-4-yl)methanesulfonamide. MS (ESI): m/z=274 [M+H]⁺.

### Synthesis of (R*)-(7-fluoro-3,3-dimethylchroman-4-yl)methanesulfonamide (Compound 44) and (S*)-(7-fluoro-3,3-dimethylchroman-4-yl)methanesulfonamide (Compound 43)

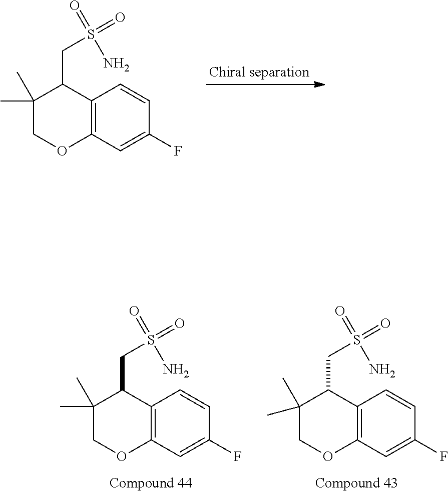 [SMILES: CC1(C)COc2cc(F)ccc2C1CS(N)(=O)=O ; "Chiral separation" ; CC1(C)COc2cc(F)ccc2[C@@H]1CS(N)(=O)=O ; CC1(C)COc2cc(F)ccc2[C@H]1CS(N)(=O)=O]

(7-fluoro-3,3-dimethyl-3,4-dihydro-2H-1-benzopyran-4-yl)methanesulfonamide was separated by chiral HPLC under the following conditions to give the two enantiomers.

Compound 44 (P1) (retention time=2.543 min), ¹H NMR (400 MHz, MeOD) δ 7.54 (dd, J=8.8, 6.4 Hz, 1H), 6.64 (td, J=8.4, 2.4 Hz, 1H), 6.48 (dd, J=10.4, 2.8 Hz, 1H), 3.84 (dd, J=14.0, 3.2 Hz, 2H), 3.53 (dd, J=14.8, 2.8 Hz, 1H), 3.25 (dd, J=14.8, 6 Hz, 1H), 3.12 (m, 1H), 1.07 (s, 3H), 1.00 (s, 3H); and Compound 43 (P2) (retention time=3.163 min)¹H NMR (400 MHz, MeOD) δ 7.54 (dd, J=8.8, 6.4 Hz, 1H), 6.64 (td, J=8.4, 2.4 Hz, 1H), 6.48 (dd, J=10.4, 2.8 Hz, 1H), 3.84 (dd, J=14.0, 3.2 Hz, 2H), 3.53 (dd, J=14.8, 2.8 Hz, 1H), 3.25 (dd, J=14.8, 6 Hz, 1H), 3.12 (m, 1H), 1.07 (s, 3H), 1.00 (s, 3H).

### Chiral HPLC Conditions:

| | |
|---|---|
| Instrument: SFC-80 (Waters) | Column: CHIRALPAK AD 20 × 250 mm, |
| | 10 um (Daicel) |
| Column temperature: 35° C. | Mobile phase: CO₂/MeOH (0.2% |
| | MeOH Ammonia) = 85/15 |
| Flow rate: 80 g/min | Back pressure: 100 bar |
| Detection wavelength: 214 nm | |
| Cycle time: 4 min | |
| Sample solution: 320 mg | Injection volume: 1.0 ml |
| dissolved in 25 mL MeOH | |

### Example 17. Preparation of (S*)-(7-fluoro-2,2-dimethylchroman-4-yl)methanesulfonamide (Compound 46) and (R*)-(7-fluoro-2,2-dimethylchroman-4-yl)methanesulfonamide (Compound 45)

Synthesis of 7-fluoro-2,2-dimethylchroman-4-one

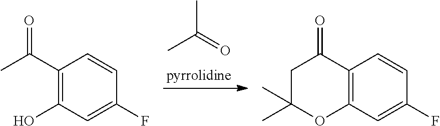

To a solution of 1-(4-fluoro-2-hydroxyphenyl)ethan-1-one (25 g, 162 mmol) in MeOH (200 mL) was added acetone (18.8 g, 324 mmol) and pyrrolidine (13.7 g, 194 mmol). After stirring at about room temperature for about 16 hours, the mixture was concentrated in vacuo. EtOAc (200 mL) and water (150 mL) were added to the residue and layers were separated. The organic layer was washed with HCl (1M aq., 50 mL) and NaHCO₃ solution (saturated, 50 mL), then dried and concentrated. The resulting substance was purified by flash column chromatography with a gradient elution of PE/EtOAc (100%:0%) to PE/EtOAc (95%:5%) to provide 7-fluoro-2,2-dimethylchroman-4-one. (ESI): m/z=195.1[M+H]⁺.

### Synthesis of tert-butyl (((7-fluoro-4-hydroxy-2,2-dimethylchroman-4-yl)methyl)sulfonyl)carbamate

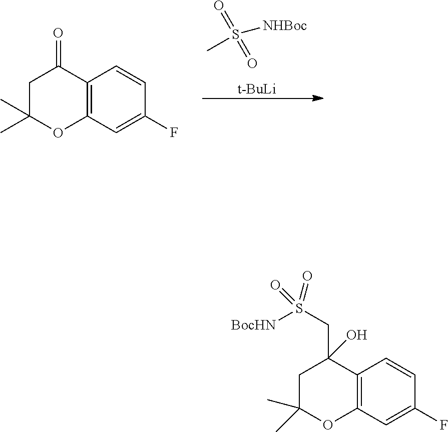 [SMILES: "t-BuLi" ; CC1(C)CC(=O)c2ccc(F)cc2O1 ; CC(C)(C)OC(=O)NS(C)(=O)=O ; CC(C)(C)OC(=O)NS(=O)(=O)CC1(O)CC(C)(C)Oc2cc(F)ccc21]

To a solution of tert-butyl N-methanesulfonylcarbamate (13.0 g, 66.8 mmol) in THF (50 mL) was added t-BuLi (1.3 M in pentane, 102 mL, 133 mmol) at about −78° C. under nitrogen. After stirring at about −78° C. for 1 h, a solution of 7-fluoro-2,2-dimethylchroman-4-one (10 g, 51.4 mmol) in THF (20 mL) was added at this temperature. The mixture was allowed to warm to about room temperature under stirring over a period of about 2 hours. Water (150 mL) was added to quench the reaction and citric acid was added to adjust the pH until pH around 4. The mixture was extracted with EtOAc (100 mL×3), organic phase dried and concentrated. The resulting substance was purified by flash column chromatography with a gradient elution of PE/EtOAc (100%:0%) to PE/EtOAc (70%:30%) to provide tert-butyl (((7-fluoro-4-hydroxy-2,2-dimethylchroman-4-yl)methyl)sulfonyl)carbamate. (ESI): m/z=412[M+Na]⁺.

### Synthesis of (7-fluoro-2,2-dimethylchroman-4-yl)methanesulfonamide

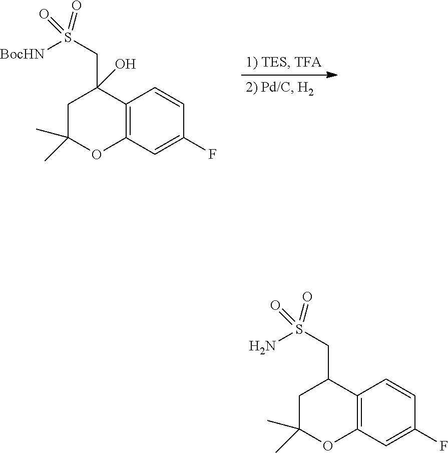 [SMILES: CC(C)(C)OC(=O)NS(=O)(=O)CC1(O)CC(C)(C)Oc2cc(F)ccc21 ; CC1(C)CC(CS(N)(=O)=O)c2ccc(F)cc2O1]

To a solution of tert-butyl (((7-fluoro-4-hydroxy-2,2-dimethylchroman-4-yl)methyl)sulfonyl)carbamate (30 g, 77.0 mmol) in DCM (80 mL) was added TES (44.6 g, 385 mmol) at 0° C. After stirring at about 0° C. for about 30 mins, TFA (87.7 g, 770 mmol) was added. The mixture was stirred at about room temperature for 16 h and the solvent was removed in vacuo. The residue was dissolved in MeOH (200 mL) and Pd/C (10% wet) (2.32 g) was added. The reaction was stirred at about room temperature for about 4 hours under H₂ atmosphere. After filtration, the filtrate was concentrated and the resulting substance was washed with EtOAc/n-hexane (1:3, 30 mL) and then acetone/H₂O (1:10, 20 mL) to give (7-fluoro-2,2-dimethylchroman-4-yl)methanesulfonamide. LCMS: ESI: m/z=274[M+H]⁺.

### Synthesis of (S*)-(7-fluoro-2,2-dimethylchroman-4-yl)methanesulfonamide (Compound 46) and (R*)-(7-fluoro-2,2-dimethylchroman-4-yl)methanesulfonamide (Compound 45)

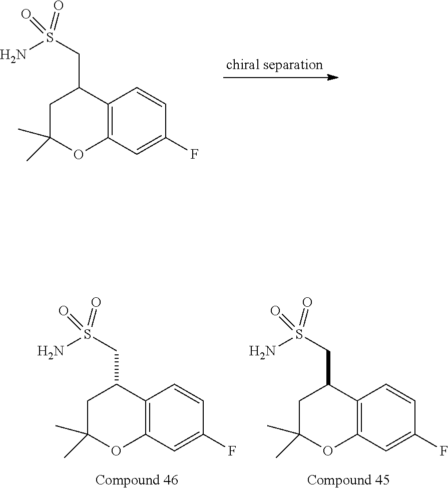

The racemic mixture of (7-fluoro-2,2-dimethylchroman-4-yl)methanesulfonamide was separated by chiral HPLC to give the two single enantiomers.

Compound 46: (P1, retention time: 1.62 min) (ESI) m/z: 274[M+H]⁺. ¹H NMR (400 MHz, CD₃OD) δ 7.34 (dd, J=7.8, 6.6 Hz, 1H), 6.64 (td, J=8.5, 2.7 Hz, 1H), 6.48 (dd, J=10.4, 2.7 Hz, 1H), 3.80 (dd, J=14.3, 3.0 Hz, 1H), 3.47 (dd, J=17.7, 9.6 Hz, 1H), 3.20 (dd, J=14.3, 9.3 Hz, 1H), 2.44 (dd, J=13.8, 6.4 Hz, 1H), 1.75 (dd, J=13.7, 11.2 Hz, 1H), 1.43 (s, 3H), 1.28 (s, 3H); and Compound 45: (P2, retention time: 2.04 min). (ESI) m/z: 274[M+H]⁺. ¹H NMR (400 MHz, CD₃OD) δ 7.34 (dd, J=7.8, 6.6 Hz, 1H), 6.64 (td, J=8.5, 2.7 Hz, 1H), 6.48 (dd, J=10.4, 2.7 Hz, 1H), 3.80 (dd, J=14.3, 3.0 Hz, 1H), 3.47 (dt, J=12.5, 9.7 Hz, 1H), 3.19 (dt, J=16.4, 8.2 Hz, 1H), 2.44 (dd, J=13.8, 6.4 Hz, 1H), 1.75 (dd, J=13.8, 11.2 Hz, 1H), 1.43 (s, 3H), 1.27 (s, 3H).

### Chiral HPLC Conditions:

| | |
|---|---|
| Instrument: SFC-150 (Thar, | Column: AD 20*250 mm, 10 um |
| Waters) | (Daicel) |
| Column temperature: 35° C. | Mobile phase: CO2/MEOH(0.2% |
| | MeOH Ammonia) = 85/15 |
| Flow rate: 100 g/min | Back pressure: 100 bar |
| Detection wavelength: 214 nm | Cycle time: 4 min |
| Sample solution: 15 g dissolved | |
| in 200 mL MeOH | |
| Injection volume: 2 mL | |

### Example 18. Preparation of (S*)-1-((R*)-7-fluoro-2,2-dimethylchroman-4-yl)ethane-1-sulfonamide (Compound 48), (R*)-1-((S*)-7-fluoro-2,2-dimethylchroman-4-yl)ethane-1-sulfonamide (Compound 47) (S*)-1-((S*)-7-fluoro-2,2-dimethylchroman-4-yl)ethane-1-sulfonamide (Compound 50) and (R*)-1-((R*)-7-fluoro-2,2-dimethylchroman-4-yl)ethane-1-sulfonamide (Compound 49)

### Synthesis of tert-butyl ((1-(7-fluoro-4-hydroxy-2,2-dimethylchroman-4-yl)ethyl)sulfonyl)carbamate

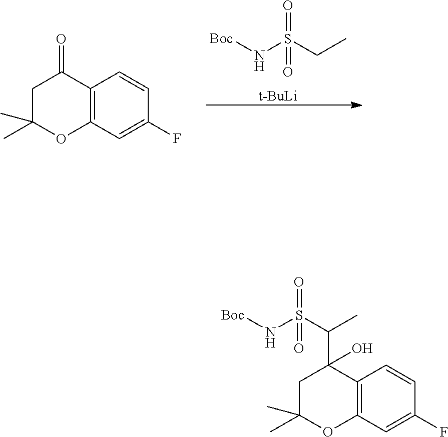 [SMILES: "t-BuLi" ; CC1(C)CC(=O)c2ccc(F)cc2O1 ; CCS(=O)(=O)NC(=O)OC(C)(C)C ; CC(C1(O)CC(C)(C)Oc2cc(F)ccc21)S(=O)(=O)NC(=O)OC(C)(C)C]

To a solution of tert-butyl (ethylsulfonyl)carbamate (8.87 g, 42.4 mmol) in THF (100 mL) was added t-BuLi (1.3 M in heptane, 54.3 ml, 70.7 mmol) at about −78° C. under N₂. After stirring at about −78° C. for about 1 hour, 7-fluoro-2, 2-dimethylchroman-4-one (5.5 g, 28.3 mmol) in THF (10 mL) was added and the mixture was gradually warmed up under stirring to about room temperature over a period of about 6 hours. Water (80 mL) was added to quench the reaction and citric acid was added to adjust the pH to 5. The mixture was extracted with EtOAc (100 mL×3), and the combined organic layers were dried and concentrated. The resulting substance was purified by silica gel column chromatography with a gradient elution of PE/EtOAc (100%:0%) to PE/EtOAc (70%:30%) to provide tert-butyl ((1-(7-fluoro-4-hydroxy-2, 2-dimethylchroman-4-yl)ethyl)sulfonyl)carbamate. MS (ESI): m/z=421 [M+H₂O]⁺.

### Synthesis of 1-(7-fluoro-2, 2-dimethylchroman-4-yl)ethane-1-sulfonamide

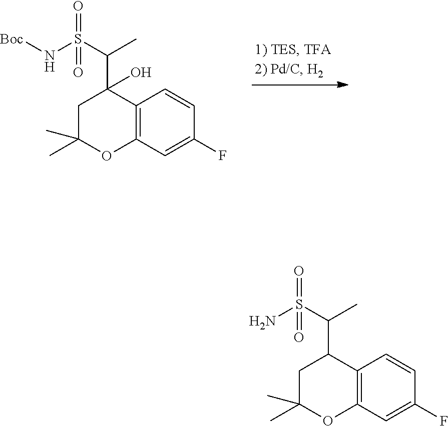 [SMILES: CC(C1(O)CC(C)(C)Oc2cc(F)ccc21)S(=O)(=O)NC(=O)OC(C)(C)C ; CC(C1CC(C)(C)Oc2cc(F)ccc21)S(N)(=O)=O]

To a solution of tert-butyl ((1-(7-fluoro-4-hydroxy-2, 2-dimethylchroman-4-yl)ethyl)sulfonyl)carbamate (2.8 g, 6.95 mmol) in DCM (50 mL) was added TES (4.0 g, 34.7 mmol) at about 0° C. After stirring at about 0° C. for about 30 min., TFA (7.9 g, 5.1 mL) was added. The reaction was stirred at about room temperature for 16 h. After concentration, the residue was dissolved in DCM (100 mL) and washed with H₂O (50 mL×2). The organic layers were dried and concentrated in vacuo. The resulting substance was dissolved in MeOH (20 mL) and Pd/C (10%, 370 mg) was added. The mixture was stirred at about room temperature under H₂ for about 2 hours. After filtration, the filtrate was concentrated, and the residue washed with PE/EtOAc (3:1, 3×10 mL) to give 1-(7-fluoro-2, 2-dimethylchroman-4-yl)ethane-1-sulfonamide. MS (ESI): m/z=309 [M+Na]⁺.

### Synthesis of (S*)-1-((R*)-7-fluoro-2,2-dimethylchroman-4-yl)ethane-1-sulfonamide (Compound 48), (R*)-1-((S*)-7-fluoro-2,2-dimethylchroman-4-yl)ethane-1-sulfonamide (Compound 47), (S*)-1-((S*)-7-fluoro-2,2-dimethylchroman-4-yl)ethane-1-sulfonamide (Compound 50) and (R*)-1-((R*)-7-fluoro-2,2-dimethylchroman-4-yl)ethane-1-sulfonamide (Compound 49)

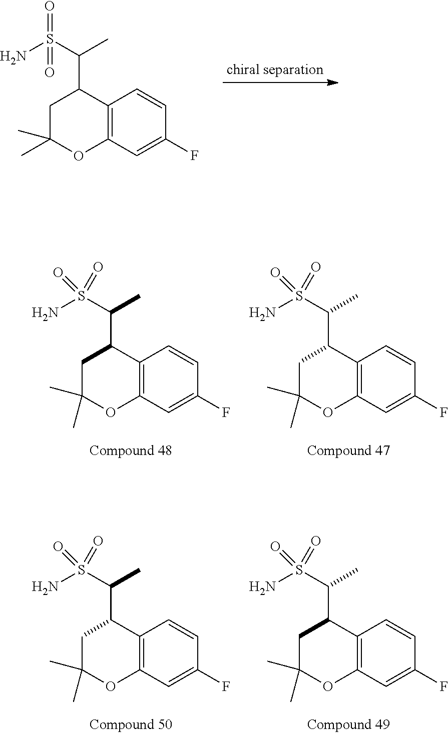 [SMILES: CC(C1CC(C)(C)Oc2cc(F)ccc21)S(N)(=O)=O ; C[C@@H]([C@@H]1CC(C)(C)Oc2cc(F)ccc21)S(N)(=O)=O ; C[C@H]([C@H]1CC(C)(C)Oc2cc(F)ccc21)S(N)(=O)=O ; C[C@@H]([C@H]1CC(C)(C)Oc2cc(F)ccc21)S(N)(=O)=O ; C[C@H]([C@@H]1CC(C)(C)Oc2cc(F)ccc21)S(N)(=O)=O]

1-(7-Fluoro-2, 2-dimethylchroman-4-yl)ethane-1-sulfonamide (a mixture of diastereomers) was separated by chiral HPLC under the following conditions. Two major diastereomers (the major pair of enantiomers) were isolated.

Compound 48 (P1) (retention time=1.30 min), MS (ESI): m/z=288, 310 [M+H, M+Na]⁺. ¹H NMR (400 MHz, CD₃OD): δ 7.71-7.67 (m, 1H), 6.61-6.56 (m, 1H), 6.48-6.45 (m, 1H), 3.68-3.63 (m, 1H), 3.60-3.54 (m, 1H), 2.02-1.91 (m, 2H), 1.44 (s, 3H), 1.31 (d, J=7.2 Hz, 3H), 1.25 (s, 3H); and Compound 47 (P2) (retention time=2.10 min), MS (ESI): m/z=288, 310 [M+H, M+Na]⁺. ¹H NMR (400 MHz, CD₃OD): 7.68-7.64 (m, 1H), 6.59-6.54 (m, 1H), 6.46-6.42 (m, 1H), 3.66-3.62 (m, 1H), 3.57-3.52 (m, 1H), 2.00-1.89 (m, 2H), 1.42 (s, 3H), 1.29 (d, J=7.2 Hz, 3H), 1.23 (s, 3H).

### Chiral HPLC Conditions:

| | |
|---|---|
| Instrument: SFC-80 (Thar, Waters) | Column: AY 20 × 250 mm, 10 μm |
| | (Daicel); |
| Column temperature: 35° C.; | Mobile phase: CO₂/EtOH (0.5% |
| | MeOH Ammonia) = 85/15; |
| Flow rate: 80 g/min; | Back pressure: 100 bar; |
| Detection wavelength: 214 nm; | Cycle time: 3.2 min; |
| Sample solution: 1500 mg | |
| dissolved in 35 ml MeOH; | |
| Injection volume: 1.0 mL. | |

### Example 19. Preparation of (R*)-2-(7-fluorochroman-4-yl)propane-2-sulfonamide (Compound 52) and (S*)-2-(7-fluorochroman-4-yl)propane-2-sulfonamide (Compound 51)

### Synthesis of 2-(7-fluoro-4-hydroxychroman-4-yl)propane-2-sulfonamide

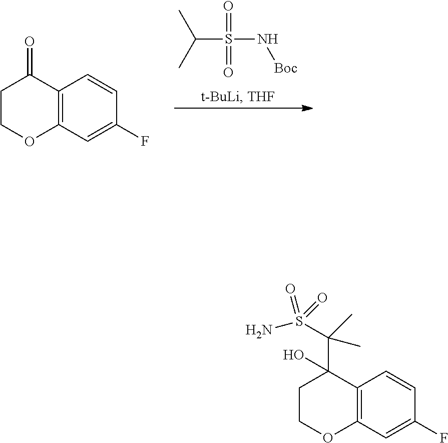

To a solution of tert-butyl N-(propane-2-sulfonyl)carbamate (2 g, 8.95 mmol) in THF (30 mL) was added t-BuLi (1.3 M in pentane, 13.7 mL, 17.9 mmol) at about −78° C. under N₂. After stirring at this temperature for about 1 hour, a solution of 7-fluoro-3,4-dihydro-2H-1-benzopyran-4-one (1.18 g, 7.15 mmol) in THF (5 mL) was added. The mixture was stirred at the same temperature for another about 2 hour. Upon completion, the mixture was quenched with water (80 mL), acidified with saturated critic acid till pH around 4 and extracted with ethyl acetate (80 mL×2). The organic layers were dried and the solvent was removed in vacuo to give the residue, which was purified by silica gel column chromatography eluted with PE/EtOAc (5:1) to yield 2-(7-fluoro-4-hydroxychroman-4-yl)propane-2-sulfonamide. MS (ESI): m/z=412[M+Na]⁺.

### Synthesis of 2-(7-fluorochroman-4-yl)propane-2-sulfonamide

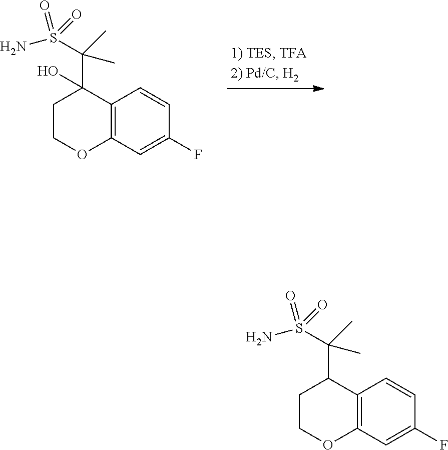 [SMILES: CC(C)(C1(O)CCOc2cc(F)ccc21)S(N)(=O)=O ; CC(C)(C1CCOc2cc(F)ccc21)S(N)(=O)=O]

To a solution of tert-butyl N-[2-(7-fluoro-4-hydroxy-3,4-dihydro-2H-1-benzopyran-4-yl)propane-2-sulfonyl]carbamate (1.3 g, 3.33 mmol) and TES (1.15 g, 9.99 mmol) in DCM (20 mL) was added TFA (3.03 g, 26.6 mmol). The mixture was stirred at about room temperature overnight. Upon completion, the solvent was removed in vacuo to give a residue, which was dissolved in acetic acid (15 mL). To the solution was added Pd/C (391 mg, 3.68 mmol). The mixture was stirred under H₂ at 60° C. overnight. Upon completion, the mixture was filtered over diatomite and the filtrate was evaporated in vacuo to give a crude product, which was purified by prep. HPLC in 0.01% aq. TFA to provide 2-(7-fluorochroman-4-yl)propane-2-sulfonamide, MS (ESI): m/z=296 [M+Na]⁺.

### Synthesis of (R*)-2-(7-fluorochroman-4-yl)propane-2-sulfonamide (Compound 52) and (S*)-2-(7-fluorochroman-4-yl)propane-2-sulfonamide (Compound 51)

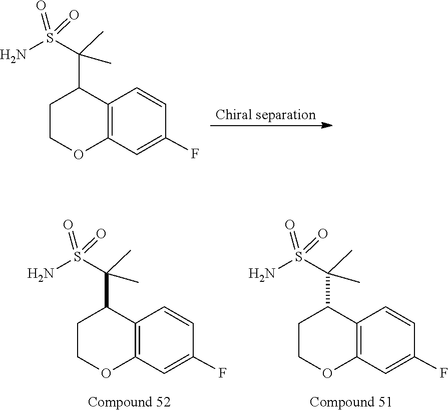

2-(7-Fluorochroman-4-yl)propane-2-sulfonamide was separated by chiral HPLC using the following conditions to give the two enantiomers.

Compound 52 (P1): (retention time: 1.45 min). MS (ESI): m/z: 296 [M+Na]⁺. ¹H NMR (400 MHz, CD₃OD): δ 7.21 (dd, J=6.8, 8.8 Hz, 1H), 6.63-6.53 (m, 2H), 4.50-4.43 (m, 1H), 4.31-4.27 (m, 1H), 3.37-3.34 (m, 1H), 2.84-2.79 (m, 1H), 2.05-1.95 (m, 1H), 1.44 (s, 3H), 1.29 (s, 3H); and Compound 51 (P2): (retention time: 1.86 min). MS (ESI): m/z: 296 [M+Na]⁺. ¹H NMR (400 MHz, CD₃OD): δ 7.21 (dd, J=6.8, 8.8 Hz, 1H), 6.63-6.53 (m, 2H), 4.50-4.43 (m, 1H), 4.31-4.27 (m, 1H), 3.37-3.34 (m, 1H), 2.84-2.79 (m, 1H), 2.05-1.95 (m, 1H), 1.44 (s, 3H), 1.29 (s, 3H).

### Chiral HPLC Conditions:

| | |
|---|---|
| Instrument: SFC-80 (Thar, | Column: AD-H 20 × 250 mm, 10 um |
| Waters) | (Daicel) |
| Column temperature: 35° C. | Mobile phase: CO₂/MeOH(0.2% |
| | MeOH Ammonia) = 80/20 |
| Flow rate: 80 g/min | Back pressure: 100 bar |
| Detection wavelength: 214 nm | Cycle time: 5.5 min |

### Example 20. Preparation of ((3S*,4R*)-6-fluoro-3-methylchroman-4-yl)methanesulfonamide (Compound 54), ((3R*,4S*)-6-fluoro-3-methylchroman-4-yl)methanesulfonamide (Compound 53), ((3S*,4S*)-6-fluoro-3-methylchroman-4-yl)methanesulfonamide (Compound 55) and ((3R*,4R*)-6-fluoro-3-methylchroman-4-yl)methanesulfonamide (Compound 56)

### Synthesis of tert-butyl(((6-fluoro-4-hydroxy-3-methylchroman-4-yl)methyl)sulfonyl) carbamate

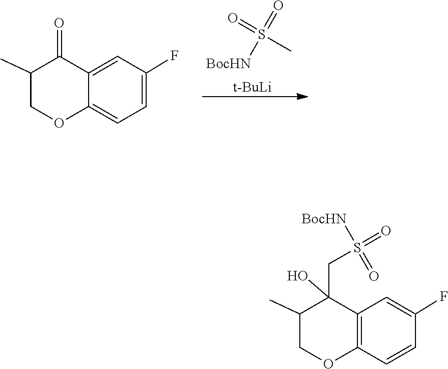 [SMILES: "t-BuLi" ; CC1COc2ccc(F)cc2C1=O ; CC(C)(C)OC(=O)NS(C)(=O)=O ; CC1COc2ccc(F)cc2C1(O)CS(=O)(=O)NC(=O)OC(C)(C)C]

To a solution of tert-butyl N-methanesulfonylcarbamate (2 g, 10.2 mmol) in THF (25 mL) was added t-BuLi (1.3 M in pentane, 15.7 mL, 20.4 mmol) at about −78° C. under N₂. After stirring at about −78° C. for about 1 hour, a solution of 6-fluoro-3-methylchroman-4-one (918 mg, 5.10 mmol) in THF (15 mL) was added. The mixture was stirred at about −78° C. for an additional about 3 hours and quenched with saturated NH₄Cl (aq. 20 mL), followed by addition of citric acid until pH around 6. The mixture was extracted with EtOAc (25 mL×2), and the combined organic layers were dried and concentrated. The resulting substance was purified by flash column chromatography with a gradient elution of DCM/MeOH (100%:0%) to DCM/MeOH (95%:5%) to provide tert-butyl (((6-fluoro-4-hydroxy-3-methylchroman-4-yl)methyl)sulfonyl) carbamate. MS (ESI): m/z=398[M+Na]⁺.

### Synthesis of (6-fluoro-3-methylchroman-4-yl)methanesulfonamide

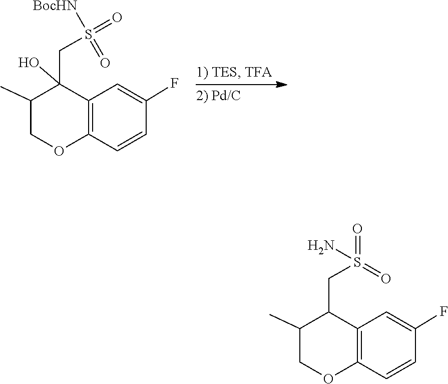 [SMILES: CC1COc2ccc(F)cc2C1(O)CS(=O)(=O)NC(=O)OC(C)(C)C ; CC1COc2ccc(F)cc2C1CS(N)(=O)=O]

To a solution of tert-butyl (((6-fluoro-4-hydroxy-3-methylchroman-4-yl)methyl)sulfonyl) carbamate (1.7 g, 4.52 mmol) in DCM (10 mL) was added TES (1.56 g, 13.5 mmol) at 0° C. After stirring at 0° C. for 30 min., TFA (6.20 g, 54.2 mmol) was added. The mixture was stirred at about room temperature for about 16 hours and concentrated. The residue was dissolved in methanol (10 mL) and Pd/C (10% wet, 949 mg) was added. The mixture was stirred at about room temperature for about 2 hours under H₂. After filtration, the filtrate was concentrated to give a crude diastereomeric mixture of (6-fluoro-3-methylchroman-4-yl)methanesulfonamide with a ratio of about 20:1 for the two pairs of enantiomers. MS (ESI): m/z=282[M+Na]⁺.

### Synthesis of ((3S*,4R*)-6-fluoro-3-methylchroman-4-yl)methanesulfonamide (Compound 54), ((3R*,4S*)-6-fluoro-3-methylchroman-4-yl)methanesulfonamide (Compound 53), ((3S*,4S*)-6-fluoro-3-methylchroman-4-yl)methanesulfonamide (Compound 55) and ((3R*,4R*)-6-fluoro-3-methylchroman-4-yl)methanesulfonamide (Compound 56)

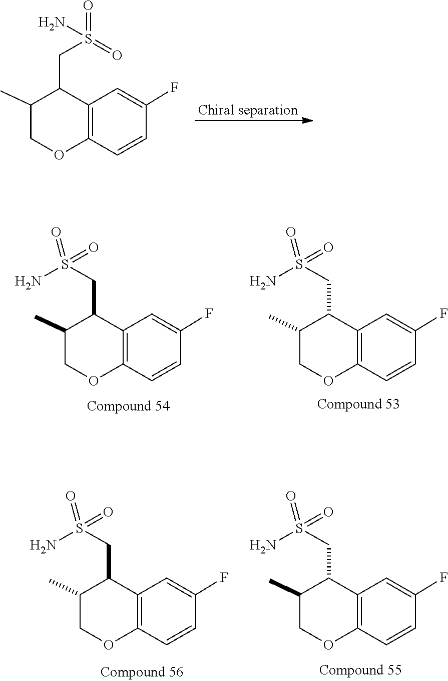

The diastereomeric mixture from Step 2 was separated by chiral HPLC. Only the major pair of two enantiomers were obtained. The minor diastereomers were lost in the separation.

Compound 54 (P1) (retention time: 1.661 min). MS (ESI): m/z 282 [M+Na]⁺. ¹H NMR (500 MHz, CD₃OD): δ 7.04-7.01 (m, 1H), 6.90-6.86 (m, 1H), 6.78-6.75 (m, 1H), 4.15-4.12 (m, 1H), 4.00-3.97 (m, 1H), 3.47-3.43 (m, 1H), 3.38-3.37 (m, 1H), 3.10-3.09 (m, 1H), 2.43-2.42 (m, 1H), 1.10 (d, J=7.1 Hz, 3H); and Compound 53 (P2) (retention time: 2.335 min). MS (ESI): m/z 282 [M+Na]⁺. ¹H NMR (500 MHz, CD₃OD): δ 7.04-7.01 (m, 1H), 6.90-6.86 (m, 1 H), 6.78-6.75 (m, 1H), 4.15-4.12 (m, 1H), 4.00-3.97 (m, 1H), 3.47-3.43 (m, 1H), 3.38-3.37 (m, 1H), 3.10-3.09 (m, 1H), 2.43-2.42 (m, 1H), 1.10 (d, J=7.1 Hz, 3H).

### Chiral HPLC Conditions:

| | |
|---|---|
| Instrument: SFC-80 (Thar, Waters) | Column: AS 20*250 mm, 10 um |
| | (Daicel) |
| Column temperature: 35° C. | |
| Mobile phase: CO2/MeOH(0.2% | |
| MeOH Ammonia) = 75/25 | |
| Flow rate: 80 g/min | Back pressure: 100 bar |
| Detection wavelength: 214 nm | Cycle time: 3.3 min |
| Sample solution: 700 mg dissolved | Injection volume: 1.9 mL |
| in 30 mL MeOH | |

### Example 21. Preparation of (R*)-(7-methylchroman-4-yl)methanesulfonamide (Compound 58) and (S*)-(7-methylchroman-4-yl)methanesulfonamide (Compound 57)

### Synthesis of tert-butyl N-[(4-hydroxy-7-methyl-3,4-dihydro-2H-1-benzopyran-4-yl)methanesulfonyl]carbamate

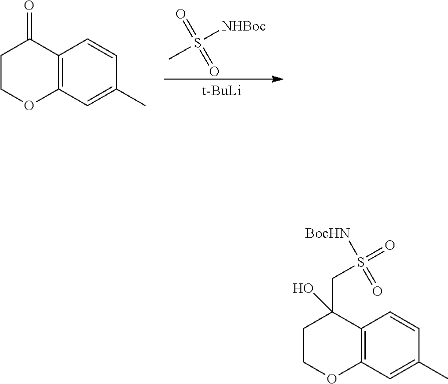 [SMILES: "t-BuLi" ; Cc1ccc2c(c1)OCCC2=O ; CC(C)(C)OC(=O)NS(C)(=O)=O ; Cc1ccc2c(c1)OCCC2(O)CS(=O)(=O)NC(=O)OC(C)(C)C]

To a solution of tert-butyl N-methanesulfonylcarbamate (960 mg, 4.92 mmol) in THF (10 mL) was added t-BuLi (1.3 M in pentane, 7.56 mL, 9.84 mmol) at about −78° C. After stirring at about −78° C. for about 40 min., 7-methylchroman-4-one (0.4 g, 2.46 mmol) in THF (2 mL) was added. The reaction was stirred at about −78° C. for about 2 h. Upon completion, water (50 mL) was added to quench the reaction and then citric acid was added to adjust the pH to about 5. The mixture was extracted with EtOAc (50 mL×2), and the combined organic layers were dried and concentrated. The resulting substance was purified by silica gel column chromatography with a gradient elution of PE/EtOAc (100%:0%) to PE/EtOAc (70%:30%) to provide tert-butyl N-[(4-hydroxy-7-methyl-3,4-dihydro-2H-1-benzopyran-4-yl)methanesulfonyl]carbamate. ESI: m/z=380[M+Na]⁺.

### Synthesis of (7-methylchroman-4-yl)methanesulfonamide

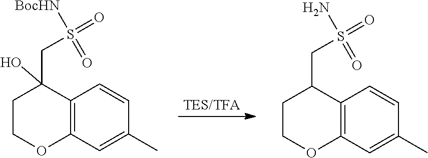 [SMILES: Cc1ccc2c(c1)OCCC2(O)CS(=O)(=O)NC(=O)OC(C)(C)C ; Cc1ccc2c(c1)OCCC2CS(N)(=O)=O]

To a solution of tert-butyl N-[(4-hydroxy-7-methyl-3,4-dihydro-2H-1-benzopyran-4-yl)methanesulfonyl]carbamate (0.7 g, 1.95 mmol) in DCM (10 mL) was added TES (679 mg, 5.84 mmol) at 0° C. After stirring at 0° C. for 20 min., TFA (2.65 g, 23.3 mmol) was added. The reaction was stirred at about room temperature for about 16 hours. Upon completion, the solvent was removed, and the residue was recrystallized from acetone and water to give (7-methylchroman-4-yl)methanesulfonamide. ESI: m/z=264[M+Na]⁺.

### Synthesis of (R*)-(7-methylchroman-4-yl)methanesulfonamide (Compound 58) and (S*)-(7-methylchroman-4-yl)methanesulfonamide (Compound 57)

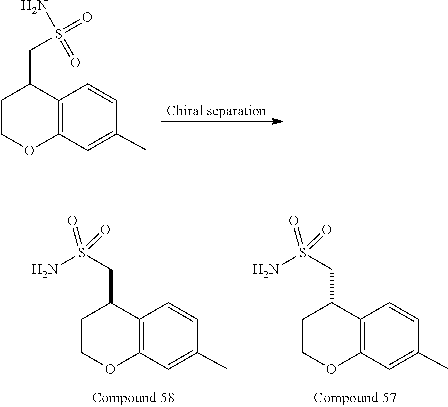

The racemic mixture of (7-methylchroman-4-yl)methanesulfonamide (260 mg) was separated by chiral HPLC using the following conditions to give the two enantiomers.

Compound 58 (retention time=1.503 min) H NMR (400 MHz, CD₃OD) δ 7.07 (d, J=7.8 Hz, 1H), 6.74-6.68 (m, 1H), 6.59 (s, 1H), 4.23-4.09 (m, 2H), 3.45 (dd, J=10.1, 4.2 Hz, 2H), 3.39-3.34 (m, 1H), 2.35-2.14 (m, 5H); and Compound 57 (retention time=1.845 min) ¹H NMR (400 MHz, CD₃OD) δ 7.08 (d, J=7.8 Hz, 1H), 6.71 (d, J=7.8 Hz, 1H), 6.59 (s, 1H), 4.24-4.10 (m, 2H), 3.45 (dd, J=10.2, 4.4 Hz, 2H), 3.39-3.34 (m, 1H), 2.47-2.01 (m, 5H).

### Chiral HPLC Conditions:

| | |
|---|---|
| Instrument: SFC-80 (Thar, | Column: IG 20*250 mm, 10 um |
| Waters) | (Daicel) |
| Column temperature: 35° C. | Mobile phase: CO₂/MeOH(0.2% |
| | MeOH Ammonia) = 70/30 |
| Flow rate: 80 g/min | Back pressure: 100 bar |
| Detection wavelength: 214 nm | Cycle time: 3.0 min |
| Sample solution: 260 mg dissolved | |
| in 25 ml MeOH | |
| Injection volume: 0.8 ml | |

### Example 22. Preparation of (R*)-(7-cyanochroman-4-yl)methanesulfonamide (Compound 60) and (S*)-(7-cyanochroman-4-yl)methanesulfonamide (Compound 59)

### Synthesis of tert-butyl (((7-bromo-4-hydroxychroman-4-yl)methyl)-sulfonyl)carbamate

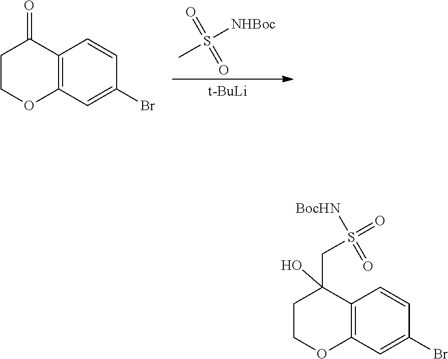 [SMILES: "t-BuLi" ; O=C1CCOc2cc(Br)ccc21 ; CC(C)(C)OC(=O)NS(C)(=O)=O ; CC(C)(C)OC(=O)NS(=O)(=O)CC1(O)CCOc2cc(Br)ccc21]

To a solution of tert-butyl (methylsulfonyl)carbamate (2.90 g, 14.9 mmol) in THF (40 mL) was added t-BuLi (1.3 M) (23.0 mL, 29.9 mmol) at about −78° C. After stirring at about −78° C. for about 1 hour, 7-bromochroman-4-one (2.27 g, 9.99 mmol) in THF (10 mL) was added. The reaction was stirred at about −78° C. for about 2 hours. Upon completion, water (80 mL) was added to quench the reaction, and then citric acid was added to adjust the pH to around 5. The mixture was extracted with EtOAc (100 mL×2), and the combined organic layers were dried and concentrated. The resulting substance was purified by silica gel column chromatography with a gradient elution of PE/EtOAc (100%:0%) to PE/EtOAc (75%:25%) to provide tert-butyl (((7-bromo-4-hydroxychroman-4-yl)methyl)sulfonyl)carbamate. ESI: m/z=444,446[M+Na]⁺.

### Synthesis of (7-bromochroman-4-yl)methanesulfonamide

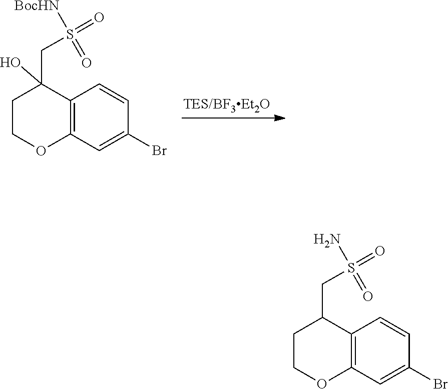 [SMILES: CC(C)(C)OC(=O)NS(=O)(=O)CC1(O)CCOc2cc(Br)ccc21 ; NS(=O)(=O)CC1CCOc2cc(Br)ccc21]

To a solution of tert-butyl (((7-bromo-4-hydroxychroman-4-yl)methyl)sulfonyl)carbamate (3.6 g, 8.52 mmol) in DCM (40 mL) was added TES (2.96 g, 25.5 mmol) at 0° C. After stirring at 0° C. for 20 mins, BF₃·Et₂O (25.5 mmol) was added and the mixture was stirred at about room temperature for about 2 hours. Upon completion, the solvent was removed, and the residue was triturated with EtOAc and PE to give 7-bromochroman-4-yl)methanesulfonamide. ESI: m/z=328, 330[M+Na]⁺.

### Synthesis of (7-cyanochroman-4-yl)methanesulfonamide

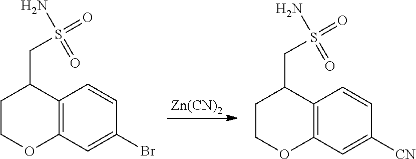

To a solution of (7-bromochroman-4-yl)methanesulfonamide (0.918 g, 2.99 mmol) in DMF (10 mL) was added zinc cyanide (702 mg, 5.98 mmol) and tetrakis(triphenylphosphine)palladium (345 mg, 299 μmol). The reaction mixture was heated to about 125° C. and stirred at that temperature for about 2 hours under microwave irradiation. Upon cooling, the mixture was washed with brine (100 mL×4), and the combined organic layers were dried and concentrated. The residue was purified by prep-HPLC to give (7-cyanochroman-4-yl)methanesulfonamide. ESI: m/z=253[M+H]⁺.

### Synthesis of (R*)-(7-cyanochroman-4-yl)methanesulfonamide (Compound 60) and (S*)-(7-cyanochroman-4-yl)methanesulfonamide (Compound 59)

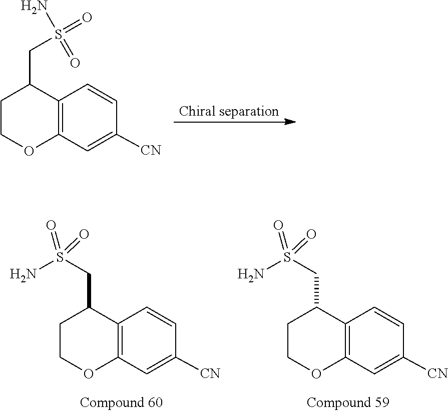

The racemic mixture of (7-methylchroman-4-yl)methanesulfonamide (330 mg) was separated by chiral HPLC using the following conditions to give the two enantiomers.

Compound 60 (retention time=1.512 min). ¹H NMR (400 MHz, CD₃OD) δ 7.42 (d, J=8.0 Hz, 1H), 7.23 (dd, J=8.0, 1.5 Hz, 1H), 7.14 (d, J=1.4 Hz, 1H), 4.31 (dt, J=11.2, 4.3 Hz, 1H), 4.28-4.19 (m, 1H), 3.55 (dt, J=8.8, 4.4 Hz, 1H), 3.45-3.32 (m, 2H), 2.39-2.30 (m, 1H), 2.9-2.20 (m, 1H); and Compound 59 (retention time=2.909 min). ¹H NMR (400 MHz, CD₃OD) δ 7.30 (d, J=8.0 Hz, 1H), 7.10 (dd, J=8.0, 1.6 Hz, 1H), 7.02 (d, J=1.5 Hz, 1H), 4.19 (dt, J=11.1, 4.3 Hz, 1H), 4.16-4.08 (m, 1H), 3.43 (dt, J=8.8, 4.5 Hz, 1H), 3.38-3.27 (m, 2H), 2.39-2.30 (m, 1H), 2.29-2.20 (m, 1H).

### Chiral Separation Conditions:

| | |
|---|---|
| Instrument: SFC-80 (Thar, | Column: IG 20*250 mm, 10 um |
| Waters) | (Daicel) |
| Column temperature: 35° C. | Mobile phase: CO₂/MeOH (0.2% |
| | MeOH Ammonia) = 45/55 |
| Flow rate: 80 g/min | Back pressure: 100 bar |
| Detection wavelength: 214 nm | Cycle time: 4.0 min |
| Sample solution: 340 mg | |
| dissolved in 25 ml MeOH | |
| Injection volume: 1.9 mL | |

### Example 23. Preparation of (4(R*))-4-(sulfamoylamino)-7-(trifluoromethyl)-3,4-dihydro-2(H)-chromene (Compound 66) and (4(S*))-4-(sulfamoylamino)-7-(trifluoromethyl)-3,4-dihydro-2(H)-chromene (Compound 65)

### Synthesis of 7-(trifluoromethyl)chroman-4-amine

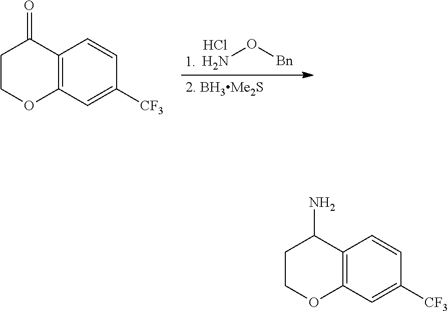 [SMILES: O=C1CCOc2cc(C(F)(F)F)ccc21 ; Cl ; NOCc1ccccc1 ; NC1CCOc2cc(C(F)(F)F)ccc21]

To a solution of 7-(trifluoromethyl)chroman-4-one (2.5 g, 11.5 mmol) in MeOH (50 mL) was added O-benzylhydroxylamine HCl salt (2.18 g, 13.7 mmol). The reaction mixture was heated to about 70° C. and stirred at this temperature for about 2 hours. The mixture was then cooled to about 0° C. Borane-dimethyl sulfide (30 mL) was added to it at about 0° C. The mixture was then heated up to about 70° C. and stirred at this temperature for about 16 hours. Upon cooling to room temperature, HCl (1M, 30 mL) was added to the reaction vessel and the pH value of the resulting mixture was adjusted to about 9 by adding slowly NaOH (aq, 2M). The mixture was extracted with DCM (3×100 mL). The combined organic layers were dried over anhydrous Na₂SO₄, filtered and concentrated in vacuo. The resulting substance was purified by silica gel column chromatography with an gradient elution of DCM/MeOH (100%:0%) to DCM/MeOH (95%:5%) to provide 7-(trifluoromethyl)chroman-4-amine. MS (ESI): m/z=201 [M+H]⁺.]

### Synthesis of 4-(sulfamoylamino)-7-(trifluoromethyl)-3,4-dihydro-2(H)-chromene

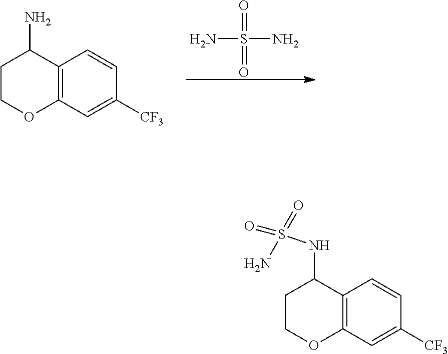 [SMILES: NC1CCOc2cc(C(F)(F)F)ccc21 ; NS(N)(=O)=O ; NS(=O)(=O)NC1CCOc2cc(C(F)(F)F)ccc21]

To a solution of 7-(trifluoromethyl) chroman-4-amine (1.1 g, 5.06 mmol) in pyridine (10 mL) was added sulfamoylamine (3.88 g, 40.4 mmol). The reaction mixture was heated to about 120° C. and stirred at that temperature for about 2 hours. Upon completion, the reaction mixture was dissolved in EtOAc (100 mL), and then washed with HCl (aq. 6M, 100 mL). The organic layer was dried and concentrated. The crude product was purified by prep-HPLC to obtained 4-(sulfamoylamino)-7-(trifluoromethyl)-3,4-dihydro-2(H)-chromene. MS (ESI): m/z=319 [M+Na]⁺.

### Synthesis of (4(R*))-4-(sulfamoylamino)-7-(trifluoromethyl)-3,4-dihydro-2(H)-chromene (Compound 66) and (4(S*))-4-(sulfamoylamino)-7-(trifluoromethyl)-3,4-dihydro-2(H)-chromene (Compound 65)

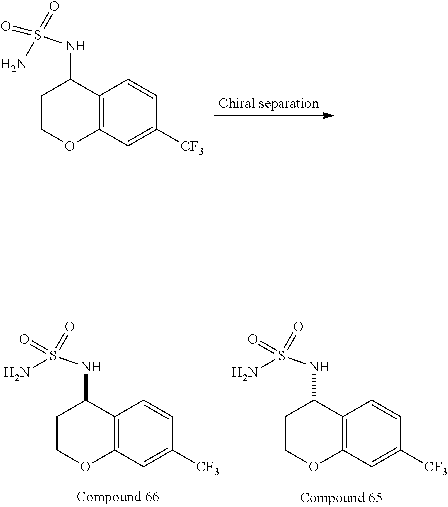 [SMILES: NS(=O)(=O)NC1CCOc2cc(C(F)(F)F)ccc21 ; NS(=O)(=O)N[C@@H]1CCOc2cc(C(F)(F)F)ccc21 ; NS(=O)(=O)N[C@H]1CCOc2cc(C(F)(F)F)ccc21]

4-(sulfamoylamino)-7-(trifluoromethyl)-3,4-dihydro-2(H)-chromene was separated by Chiral HPLC under the following conditions to provide the two enantiomers:

Compound 65 (P1) (retention time=1.25 min). MS (ESI): m/z=319 [M+Na]⁺. ¹H NMR (400 MHz, CD₃OD) δ 7.68-7.66 (d, J=8.1 Hz, 1H), 7.18-7.16 (d, J=7.9 Hz, 1H), 7.042 (s, 1H), 4.66-4.63 (m, 1H), 4.33-4.30 (t, J=5.4 Hz, 2H), 2.30-2.31 (m, 2H); and Compound 66 (P2) (retention time=1.95 min). MS (ESI): m/z=319 [M+Na]⁺. ¹H NMR (400 MHz, CD₃OD) δ 7.68-7.66 (d, J=8.1 Hz, 1H), 7.19-7.16 (dd, J=8.1, 1.2 Hz, 1H), 7.04 (s, 1H), 4.66-4.63 (t, J=5.6 Hz, 1H), 4.33-4.30 (m, 2H), 2.30-2.21 (m, 2H).

### Chiral HPLC Conditions:

| | |
|---|---|
| Instrument: SFC-150 | Column: AD 20 × 250 mm, 10 um |
| (Waters) | (Daicel) |
| Column temperature: 35° C. | Mobile phase: CO₂/MeOH (0.2% |
| | MeOH Ammonia) = 70/30 |
| Flow rate: 120 g/min | Back pressure: 100 bar |
| Detection wavelength: 214 nm | Cycle time: 6 min |
| Sample solution: 760 mg dissolved | |
| in 30 mL MeOH | |
| Injection volume: 2 mL | |

### Example 24. Preparation of (4(R*))-7-fluoro-2,2-dimethyl-4-(sulfamoylamino)-3,4-dihydrochromene (Compound 68) and (4(S*))-7-fluoro-2,2-dimethyl-4-(sulfamoylamino)-3,4-dihydrochromene (Compound 67)

### Synthesis of 7-fluoro-2,2-dimethylchroman-4-amine

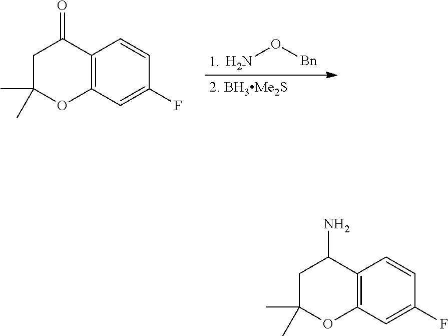

To a solution of 7-fluoro-2,2-dimethylchroman-4-one (3 g, 15.4 mmol) in MeOH (50 mL) was added O-benzylhydroxylamine (2.93 g, 18.4 mmol). The reaction mixture was heated to about 70° C. and stirred at that temperature for about 2 hours. Upon completion, the mixture was concentrated and BH₃·Me₂S (2 N in THF, 40 mL) was slowly added at about 0° C. The mixture was then stirred at about 70° C. for about 16 hours. Upon cooling to about room temperature, HCl (aq. 1M, 30 mL) was added to the reaction vessel and then NaOH (2N, aq.) was slowly added to adjust the pH to around 9. The mixture was extracted with DCM (3×100 mL) and the combined organics were dried and concentrated in vacuo. The residue was purified by silica gel chromatography with a gradient elution of DCM/MeOH (100%:0%) to DCM/MeOH (95%:5%) to provide 7-fluoro-2,2-dimethylchroman-4-amine. MS (ESI): m/z=179 [M-NH₂]⁺.

### Synthesis of 7-fluoro-2,2-dimethyl-4-(sulfamoylamino)-3,4-dihydrochromene

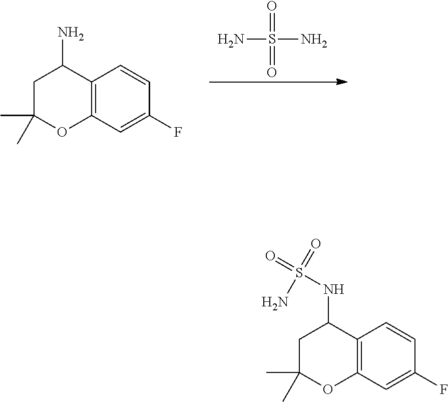 [SMILES: CC1(C)CC(N)c2ccc(F)cc2O1 ; NS(N)(=O)=O ; CC1(C)CC(NS(N)(=O)=O)c2ccc(F)cc2O1]

To a solution of 7-fluoro-2,2-dimethylchroman-4-amine (0.686 g, 3.51 mmol) in pyridine (8 mL) was added sulfamoylamine (2.69 g, 28.0 mmol). The reaction mixture was heated to about 120° C. and stirred at that temperature for about 2 hours. Upon cooling to room temperature, DCM (30 mL) and HCl (1 N, aq, 30 mL) were added. The organic phase was separated and concentrated. The residue was purified by pre-HPLC to give 7-fluoro-2,2-dimethyl-4-(sulfamoylamino)-3,4-dihydrochromene. MS (ESI): m/z=297[M+Na]⁺.

### Synthesis of (4(R*))-7-fluoro-2,2-dimethyl-4-(sulfamoylamino)-3,4-dihydrochromene (Compound 68) and (4(S*))-7-fluoro-2,2-dimethyl-4-(sulfamoylamino)-3,4-dihydrochromene (Compound 67)

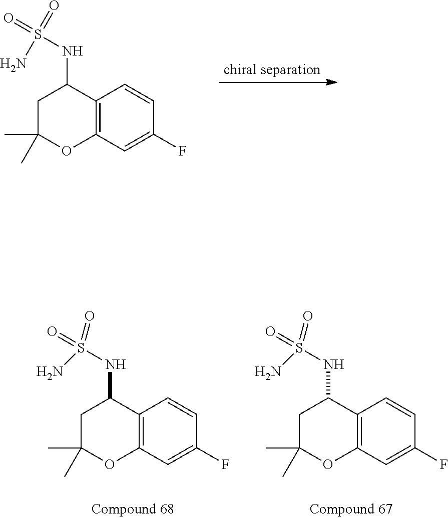

The racemic mixture of 7-fluoro-2,2-dimethyl-4-(sulfamoylamino)-3,4-dihydrochromene was separated by chiral HPLC by using the following conditions to give the two enantiomers.

Compound 67 (P1) (retention time=1.63 min). MS (ESI): m/z=297[M+23]⁺. ¹H NMR (400 MHz, CD₃OD) δ 7.61-7.57 (m, 1H), 6.66-6.61 (m, 1H), 6.46-6.43 (dd, J=10.4, 2.6 Hz, 1H), 4.60-4.56 (dd, J=11.1, 6.1 Hz, 1H), 2.40-2.35 (dd, J=13.3, 6.1 Hz, 1H), 1.87-1.81 (m, 1H), 1.44 (s, 3H), 1.32 (s, 3H); and Compound 68 (P2) (retention time=2.07 min). MS (ESI): m/z=297[M+23]⁺. ¹H NMR (400 MHz, CD₃OD) δ 7.61-7.57 (m, 1H), 6.66-6.61 (m, 1H), 6.46-6.43 (dd, J=10.4, 2.6 Hz, 1H), 4.60-4.56 (dd, J=11.2, 6.1 Hz, 1H), 2.40-2.35 (dd, J=13.3, 6.1 Hz, 1H), 1.87-1.81 (m, 1H), 1.44 (s, 3H), 1.32 (s, 3H).

### Chiral HPLC Conditions:

| | |
|---|---|
| Instrument: SFC-80 (Thar, | Column: OJ 20*250 mm, 10 um |
| Waters) | (Daicel) |
| Column temperature: 35° C. | Mobile phase: CO₂/MeOH(0.2% |
| | MeOH Ammonia) = 85/15 |
| Flow rate: 80 g/min | Back pressure: 100 bar |
| Detection wavelength: 214 nm | Cycle time: 3.9 min |
| Sample solution: 550 mg dissolved | |
| in 30 mL MeOH | |
| Injection volume: 1.0 mL | |

### Example 25. Preparation of 4(R*))-7-fluoro-4[methyl(sulfamoyl)amino]-3,4-dihydro-2(H)-chromene (Compound 70) and 4(S*))-7-fluoro-4-[methyl(sulfamoyl)amino]-3,4-dihydro-2(H)-chromene (Compound 69)

### Synthesis of 7-fluorochroman-4-amine

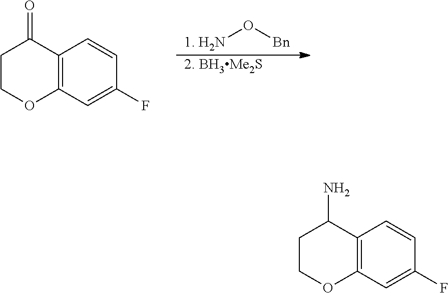

To a solution of 7-fluorochroman-4-one (2.0 g, 12.0 mmol) in MeOH (30 mL) was added O-benzylhydroxylamine HCl salt (2.28 g, 14.3 mmol) at about room temperature. The mixture was stirred at 70° C. for 2 h. Upon cooling to about 0° C., BH₃·Me₂S (20 ml) was slowly added to the mixture. The mixture was stirred at about 70° C. for about 16 hours. Upon cooling to room temperature, HCl (1M, aq. 30 mL) was added to the reaction vessel. NaOH (2N, aq.) was slowly added to adjust the pH to around 9. The mixture was extracted with DCM (30 mL×3) and the combined organic layer was dried and concentrated in vacuo. The resulting residue was purified by silica gel column chromatography with a gradient elution of DCM/MeOH (100%:0%) to DCM/MeOH (95%:5%) to provide 7-fluorochroman-4-amine. MS (ESI): m/z=151[M-NH₂]⁺.

### Synthesis of 7-fluoro-N-methylchroman-4-amine

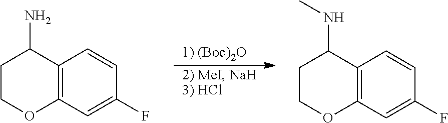

To a solution of 7-fluorochroman-4-amine (1.1 g, 6.57 mmol) in DCM (25 mL) was added di-tert-butyl dicarbonate (1.71 g, 7.88 mmol) and TEA (1.98 g, 19.7 mmol). The reaction was stirred at about room temperature for about 2 hours. Upon completion, the mixture was washed with water (100 mL×2), and the combined organic layers were dried and concentrated. The resulting substance was purified by silica gel column chromatography with a gradient elution of PE/EtOAc (100%:0%) to PE/EtOAc (92%:8%) to provide tert-butyl (7-fluorochroman-4-yl)carbamate. ESI: m/z=290[M+Na]⁺.

To a solution of tert-butyl (7-fluorochroman-4-yl)carbamate (1.7 g, 6.35 mmol) in DMF (10 mL) was added NaH (60%) (508 mg, 12.7 mmol) at about 0° C. The mixture was stirred at about 0° C. for about 1 hour and iodomethane (1.35 g, 9.52 mmol) was added. After stirring at about room temperature for 2 h, water (80 mL) and EtOAc (150 mL) were added. The mixture was washed with brine (100 mL×4), the combined organic layers were dried and concentrated. The residue was purified by silica gel column chromatography with a gradient elution of PE/EtOAc (100%:0%) to PE/EtOAc (93%:7%) to provide tert-butyl N-(7-fluoro-3,4-dihydro-2H-1-benzopyran-4-yl)-N-methylcarbamate. ESI: m/z=304[M+Na]⁺.

To a solution of tert-butyl N-(7-fluoro-3,4-dihydro-2H-1-benzopyran-4-yl)-N-methylcarbamate (0.85 g, 3.02 mmol) in EtOAc (10 mL) was added HCl/EtOAc (3M, 10 mL). The reaction was stirred at about room temperature for about 3 hours and the solvent was removed. The residue was dissolved in water (30 mL), neutralized with NaOH (2N) to pH around 10, and then extracted with DCM (50 mL×2). The organic phase was dried and concentrated to give 7-fluoro-N-methylchroman-4-amine. ESI: m/z=151[M−30]⁺.

### Synthesis of benzyl (N-(7-fluorochroman-4-yl)-N-methylsulfamoyl)carbamate

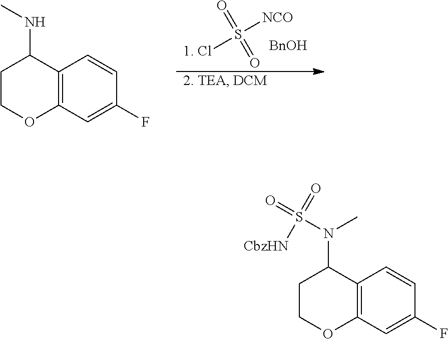

To a solution of [(chlorosulfonyl)imino]methanone (778 mg, 5.5 mmol) in DCM (10 mL) was added benzyl alcohol (594 mg, 5.5 mmol) at about 0° C. The mixture was stirred at about room temperature for about 0.5 hours and poured into a solution of 7-fluoro-N-methylchroman-4-amine (0.5 g, 2.75 mmol) and TEA (555 mg, 5.5 mmol) in DCM (10 mL) at 0° C. The mixture was stirred at this temperature for another about 1 hour and water (30 mL) was added. Citric acid was added slowly to adjust the pH to around 5. The mixture was extracted with DCM (50 mL×2), organic phase dried and concentrated. The residue was purified by prep-HPLC to give benzyl (N-(7-fluorochroman-4-yl)-N-methylsulfamoyl)carbamate. (ESI) m/z: 417[M+Na]⁺.

### Synthesis of N-(7-fluoro-3,4-dihydro-2H-1-benzopyran-4-yl)-N-methylaminosulfonamide

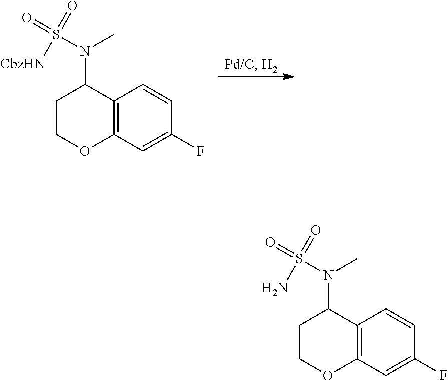 [SMILES: CN(C1CCOc2cc(F)ccc21)S(=O)(=O)NC(=O)OCc1ccccc1 ; "Pd/C, H2" ; CN(C1CCOc2cc(F)ccc21)S(N)(=O)=O]

To a solution of benzyl (N-(7-fluorochroman-4-yl)-N-methylsulfamoyl)carbamate (0.74 g, 1.87 mmol) in methanol (20 mL) was added Pd/C (10%) (196 mg, 1.87 mmol). The reaction was stirred at about room temperature for about 2 hours under H₂. Upon completion, the mixture was filtered and the filtrate concentrated in vacuo. The residual crude racemate N-(7-fluoro-3,4-dihydro-2H-1-benzopyran-4-yl)-N-methylaminosulfonamide (0.44 g) was used for next step without further purification. ESI: m/z=283[M+Na]⁺.

### Synthesis of 4(R*))-7-fluoro-4-[methyl(sulfamoyl)amino]-3,4-dihydro-2(H)-chromene (Compound 70) and 4(S*))-7-fluoro-4-[methyl(sulfamoyl)amino]-3,4-dihydro-2(H)-chromene (Compound 69)

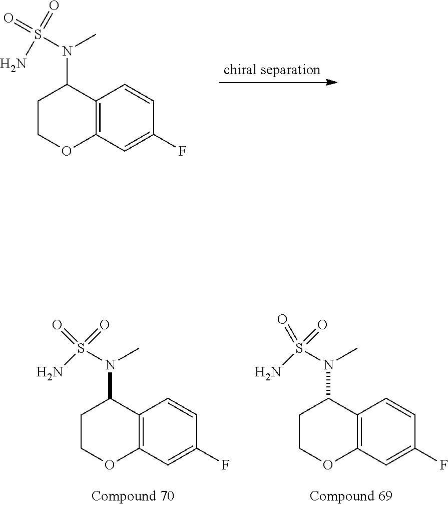 [SMILES: CN(C1CCOc2cc(F)ccc21)S(N)(=O)=O ; CN([C@@H]1CCOc2cc(F)ccc21)S(N)(=O)=O ; CN([C@H]1CCOc2cc(F)ccc21)S(N)(=O)=O]

N-(7-fluoro-3,4-dihydro-2H-1-benzopyran-4-yl)-N-methylaminosulfonamide (0.44 g) was separated by chiral HPLC using the following conditions to give the two enantiomers.

Compound 70 (P1) (retention time=1.082 min) ¹H NMR (500 MHz, CD₃OD) δ 7.41-7.34 (m, 1H), 6.68 (td, J=8.5, 2.6 Hz, 1H), 6.53 (dd, J=10.3, 2.6 Hz, 1H), 5.17 (t, J=8.1 Hz, 1H), 4.36 (dt, J=11.3, 3.9 Hz, 1H), 4.21-4.17 (m, 1H), 2.59 (s, 3H), 2.25-2.20 (m, 2H); and Compound 69 (P2) (retention time=1.895 min) ¹H NMR (500 MHz, CD₃OD) δ 7.41-7.34 (m, 1H), 6.68 (td, J=8.5, 2.6 Hz, 1H), 6.53 (dd, J=10.3, 2.6 Hz, 1H), 5.17 (t, J=8.1 Hz, 1H), 4.36 (dt, J=11.3, 3.9 Hz, 1H), 4.21-4.17 (m, 1H), 2.59 (s, 3H), 2.25-2.20 (m, 2H)

### Chiral HPLC Conditions:

| | |
|---|---|
| Instrument: SFC-80 (Thar, | Column: IG 20*250 mm, 10 um |
| Waters) | (Daicel) |
| Column temperature: 35° C. | Mobile phase: CO₂/MeOH(0.2% |
| | MeOH Ammonia) = 55/45 |
| Flow rate: 80 g/min | Back pressure: 100 bar |
| Detection wavelength: 214 nm | Cycle time: 3.5 min |
| Sample solution: 440 mg dissolved | |
| in 35 ml MeOH | |
| Injection volume: 1.9 ml | |

### Example 26. Preparation of 4(R*))-7-fluoro-4-(sulfamoylamino)-3,4-dihydro-2(H)-chromene (Compound 72) and 4(S*))-7-fluoro-4-(sulfamoylamino)-3,4-dihydro-2(H)-chromene (Compound 71)

### Synthesis of 7-fluoro-4-(sulfamoylamino)-3,4-dihydro-2(H)-chromene

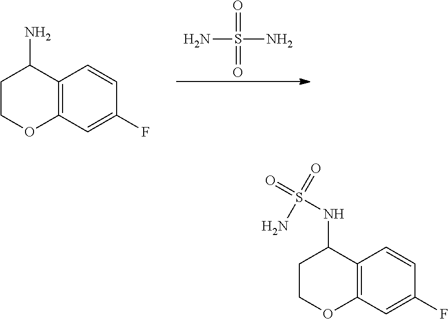 [SMILES: NC1CCOc2cc(F)ccc21 ; NS(N)(=O)=O ; NS(=O)(=O)NC1CCOc2cc(F)ccc21]

To a solution of 7-fluorochroman-4-amine (6 g, 35.8 mmol) in pyridine (100 mL) was added sulfamoylamine (27.4 g, 286 mmol). The reaction mixture was heated to about 120° C. and stirred at that temperature for about 1 hour. Upon cooling, the mixture was poured into water (100 mL) and filtered. The filter cake was rinsed with water (30 mL) to give 7-fluoro-4-(sulfamoylamino)-3,4-dihydro-2(H)-chromene. MS (ESI): m/z=269[M+Na]⁺.

### Synthesis of 4(R*))-7-fluoro-4-(sulfamoylamino)-3,4-dihydro-2(H)-chromene (Compound 72) and 4(S*))-7-fluoro-4-(sulfamoylamino)-3,4-dihydro-2(H)-chromene (Compound 71)

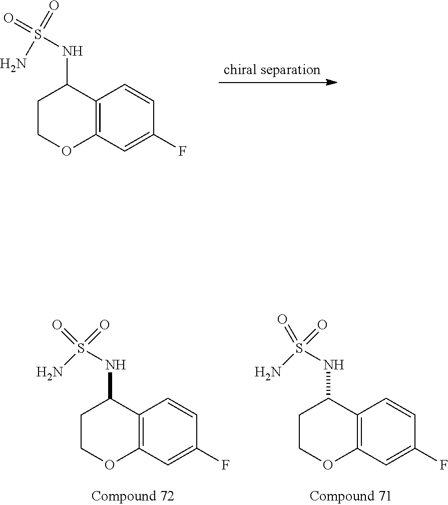 [SMILES: NS(=O)(=O)NC1CCOc2cc(F)ccc21 ; NS(=O)(=O)N[C@@H]1CCOc2cc(F)ccc21 ; NS(=O)(=O)N[C@H]1CCOc2cc(F)ccc21]

The racemic mixture of 7-fluoro-4-(sulfamoylamino)-3,4-dihydro-2(H)-chromene (4 g) was separated by chiral HPLC using the following conditions to give the two enantiomers.

Compound 72 (P1) (retention time: 1.066 min). MS (ESI): m/z 269 [M+Na]⁺. ¹H NMR (500 MHz, CD₃OD) δ: 7.47 (dd, J=8.5, 6.8 Hz, 1H), 6.66 (td, J=8.5, 2.6 Hz, 1H), 6.51 (dd, J=10.4, 2.6 Hz, 1H), 4.56 (t, J=5.0 Hz, 1H), 4.34-4.22 (m, 2H), 2.21 (dd, J=10.6, 5.2 Hz, 2H); and Compound 71 (P2) (retention time: 3.211 min). MS (ESI): m/z 269 [M+Na]⁺. ¹H NMR (500 MHz, CD₃OD) δ: 7.47 (dd, J=8.5, 6.8 Hz, 1H), 6.66 (td, J=8.5, 2.6 Hz, 1H), 6.51 (dd, J=10.4, 2.6 Hz, 1H), 4.56 (t, J=5.0 Hz, 1H), 4.34-4.22 (m, 2H), 2.21 (dd, J=10.6, 5.2 Hz, 2H).

### Chiral HPLC Conditions:

| | |
|---|---|
| Instrument: SFC-80 (Thar, | Column: IG 20*250 mm, 10 um |
| Waters) | (Daicel) |
| Column temperature: 40° C. | Mobile phase: CO₂/MEOH (0.2% |
| | MeOH Ammonia) = 40/60 |
| Flow rate: 80 g/min | Back pressure: 100 bar |
| Detection wavelength: 214 nm | Cycle time: 6 min |
| Sample solution: 4 g dissolved | |
| in 200 ml | |
| Methanol Injection volume: 4 ml | |

### Example 27. Preparation of (3(R*),4(S*))-7-fluoro-3-methyl-4-(sulfamoylamino)-3,4-dihydro-2(H)-chromene (Compound 74), (3(S*),4(R*))-7-fluoro-3-methyl-4-(sulfamoylamino)-3,4-dihydro-2(H)-chromene (Compound 73), (3(R*),4(R*))-7-fluoro-3-methyl-4-(sulfamoylamino)-3,4-dihydro-2(H)-chromene (Compound 75) and (3(S*),4(S*))-7-fluoro-3-methyl-4-(sulfamoylamino)-3,4-dihydro-2(H)-chromene (Compound 76)

### Synthesis of N-(7-fluoro-3-methylchroman-4-yl)-2-methyl propane-2-sulfinamide

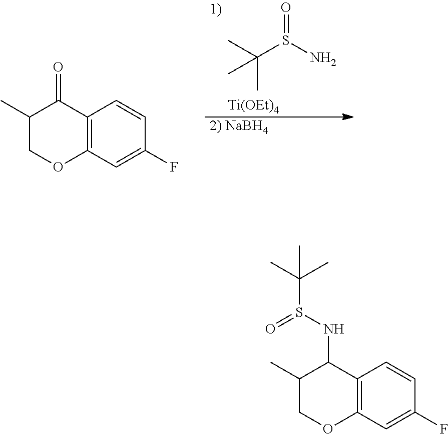

To a solution of 7-fluoro-3-methylchroman-4-one (2.0 g, 11.1 mmol) in THF (30 mL) was added 2-methylpropane-2-sulfinamide (2.69 g, 22.2 mmol) and titanium ethoxide (7.59 g, 33.3 mmol) under nitrogen. The mixture was heated to reflux with stirring overnight. Upon completion, the mixture was quenched with water (200 mL) and EtOAc (100 mL) was added. The substance was removed by filtration and the layers were separated. The organic layer was dried and concentrated under in vacuo. The residue was dissolved in MeOH (30 mL) and sodium borohydride (596 mg, 15.7 mmol) was added at about 0° C. The mixture was stirred at this temperature for 1 h. Upon completion, the mixture was quenched with water (150 mL), evaporated in vacuo to remove the methanol, and extracted with EtOAc (100 mL×2). The organic layer was separated, dried, and concentrated in vacuo. The crude product was purified by silica gel column chromatography eluted with PE/EtOAc (5/1) to give N-(7-fluoro-3-methylchroman-4-yl)-2-methyl propane-2-sulfinamide. (ESI) m/z: 286[M+H]⁺.

### Synthesis of 7-fluoro-3-methylchroman-4-amine

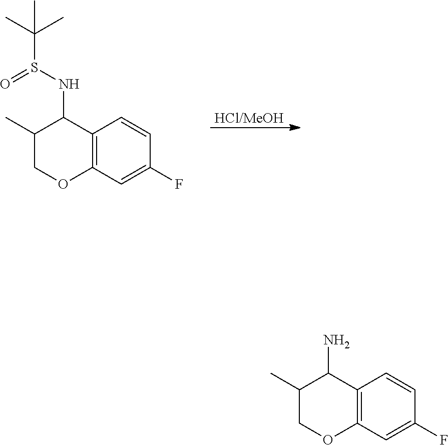 [SMILES: CC1COc2cc(F)ccc2C1NS(=O)C(C)(C)C ; "HCl/MeOH" ; CC1COc2cc(F)ccc2C1N]

To a solution of N-(7-fluoro-3-methylchroman-4-yl)-2-methyl propane-2-sulfinamide (3.0 g, 10.5 mmol) in MeOH (10 mL) was added HCl in MeOH (3N, 21 mL, 63.0 mmol). The mixture was stirred at about room temperature overnight and then evaporated in vacuo to dryness. Water (150 mL) was added and the mixture was washed with PE/EtOAc (2:1) (100 mL), basified with NaOH (15% aq.) till pH around 12, and extracted with EtOAc (100 mL×2). The organic layer was separated, dried and concentrated in vacuo to give 7-fluoro-3-methylchroman-4-amine. (ESI) m/z: 165[M-NH₂]⁺.

### Synthesis of tert-butyl (N-(7-fluoro-3-methylchroman-4-yl) sulfamoyl)carbamate

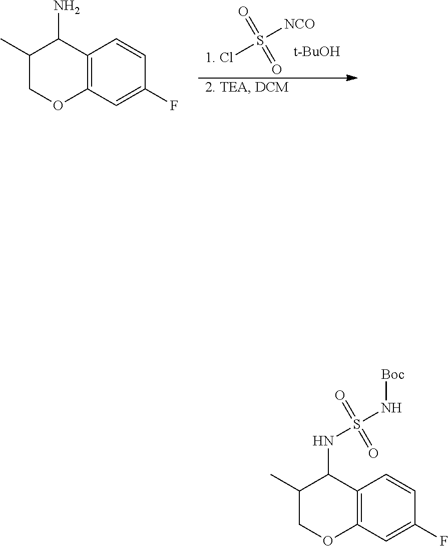

To a solution of [(chlorosulfonyl)imino]methanone (1.67 g, 11.8 mmol) in DCM (15 mL) was added 2-methylpropan-2-ol (873 mg, 11.8 mmol) at 0° C. The mixture was stirred at about room temperature for about 0.5 hours and then dropwise added into a solution of 7-fluoro-3-methyl-3,4-dihydro-2H-1-benzopyran-4-amine (1.43 g, 7.89 mmol) and TEA (1.27 g, 12.6 mmol) in DCM (15 mL) at 0° C. After addition, the mixture was stirred at this temperature for another about 1 hour, quenched with saturated critic acid (aq. 50 mL), and extracted with dichloromethane (50 mL×2). The organic phase was dried and concentrated in vacuo. The residue was triturated with Et₂O/DCM (8/1) to give tert-butyl (N-(7-fluoro-3-methylchroman-4-yl) sulfamoyl)carbamate, (ESI) m/z: 383[M+Na]⁺.

### Synthesis of 7-fluoro-3-methyl-4-(sulfamoylamino)-3,4-dihydro-2(H)-chromene

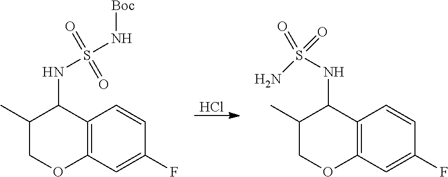 [SMILES: CC1COc2cc(F)ccc2C1NS(=O)(=O)NC(=O)OC(C)(C)C ; Cl ; CC1COc2cc(F)ccc2C1NS(N)(=O)=O]

To a solution of tert-butyl (N-(7-fluoro-3-methylchroman-4-yl) sulfamoyl)carbamate (2.1 g, 5.82 mmol) in DCM (15 mL) was added HCl in dioxane (4N, 14.5 mL, 58.2 mmol) at about room temperature. The mixture was stirred at this temperature overnight and concentrated in vacuo. The crude product was purified by silica gel column chromatography eluted with DCM/MeOH (20:1) to give 7-fluoro-3-methyl-4-(sulfamoylamino)-3,4-dihydro-2(H)-chromene as a mixture of diastereomers. (ESI) m/z: 283[M+Na]⁺.

### Synthesis of (3(R*),4(S*))-7-fluoro-3-methyl-4-(sulfamoylamino)-3,4-dihydro-2(H)-chromene (Compound 74), (3(S*),4(R*))-7-fluoro-3-methyl-4-(sulfamoylamino)-3,4-dihydro-2(H)-chromene (Compound 73), (3(R*),4(R*))-7-fluoro-3-methyl-4-(sulfamoylamino)-3,4-dihydro-2(H)-chromene (Compound 75) and (3(S*),4(S*))-7-fluoro-3-methyl-4-(sulfamoylamino)-3,4-dihydro-2(H)-chromene (Compound 76)

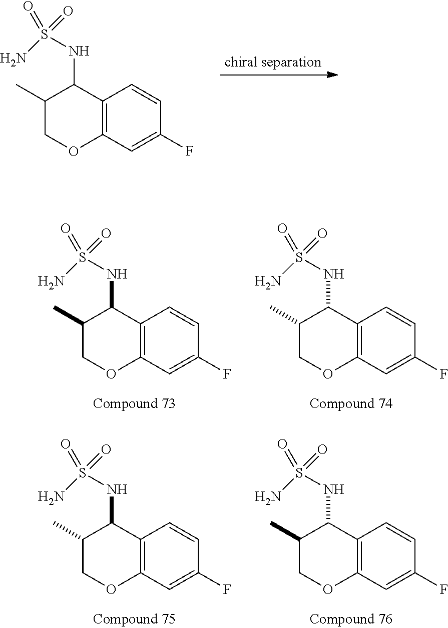 [SMILES: CC1COc2cc(F)ccc2C1NS(N)(=O)=O ; C[C@@H]1COc2cc(F)ccc2[C@@H]1NS(N)(=O)=O ; C[C@H]1COc2cc(F)ccc2[C@H]1NS(N)(=O)=O ; C[C@H]1COc2cc(F)ccc2[C@@H]1NS(N)(=O)=O ; C[C@@H]1COc2cc(F)ccc2[C@H]1NS(N)(=O)=O]

The diastereomeric mixture of 7-fluoro-3-methyl-4-(sulfamoylamino)-3,4-dihydro-2(H)-chromene was separated by chiral HPLC using the following conditions to give the four diastereomers.

Compound 75: P1 (retention time: 0.866). (ESI) m/z: 283[M+Na]⁺. ¹H-NMR (CD₃OD): δ 7.52-7.48 (m, 1H), 6.70-6.65 (m, 1H), 6.53-6.49 (m, 1H), 4.27-4.24 (m, 1H), 4.17 (d, J=4.8 Hz, 1H), 4.02-3.98 (m, 1H), 2.28-2.26 (m, 1H), 1.07 (d, J=7.2 Hz, 3H); Compound 73: P2 (retention time: 1.001). (ESI) m/z: 283[M+Na]⁺. ¹H-NMR (CD₃OD): δ 7.59-7.56 (m, 1H), 6.67-6.63 (m, 1H), 6.51-6.49 (m, 1H), 4.57 (d, J=3.6 Hz, 1H), 4.15-4.12 (m, 1H), 3.94-3.90 (m, 1H), 2.32-2.27 (m, 1H), 1.11 (d, J=5.2 Hz, 3H); Compound 76: P3 (retention time: 1.300). (ESI) m/z: 283[M+Na]⁺. ¹H-NMR (CD₃OD): δ 7.52-7.48 (m, 1H), 6.70-6.65 (m, 1H), 6.53-6.49 (m, 1H), 4.27-4.24 (m, 1H), 4.17 (d, J=4.8 Hz, 1H), 4.02-3.98 (m, 1H), 2.28-2.26 (m, 1H), 1.07 (d, J=7.2 Hz, 3H); and Compound 74: P4 (retention time: 1.680). (ESI) m/z: 283[M+Na]⁺. ¹H-NMR (CD₃OD): δ 7.59-7.56 (m, 1H), 6.67-6.63 (m, 1H), 6.51-6.49 (m, 1H), 4.57 (d, J=3.6 Hz, 1H), 4.15-4.12 (m, 1H), 3.94-3.90 (m, 1H), 2.32-2.27 (m, 1H), 1.11 (d, J=5.2 Hz, 3H).

### Chiral HPLC Conditions:

| | |
|---|---|
| Instrument: SFC-80 (Thar, | Column: IG 20*250 mm, 10 um |
| Waters) | (Daicel) |
| Column temperature: 35° C. | Mobile phase: CO₂/MeOH(0.2% |
| | MeOH Ammonia) = 80/20 |
| Flow rate: 80 g/min | Back pressure: 100 bar |
| Detection wavelength: 214 nm | Cycle time: 3.1 min |

Assays

Exemplary compounds disclosed herein were tested in the Harmaline-induced Tremor Model and/or Tacrine-induced Tremor.

Harmaline-induced Tremor: Harmaline-induced tremor is widely recognized as an animal model of Essential Tremor (ET). Following acute administration, harmaline produces an 8-16 Hz tremor in rodents, cats, and monkeys that, like ET, is postural and kinetic, involving appendicular and axial musculature (Handforth, A. (2012). Harmaline tremor: underlying mechanisms in a potential animal model of essential tremor. Tremor and Other Hyperkinetic Movements (NY), 2, 02-92-769-1; Handforth, A. (2016). Linking Essential Tremor to the Cerebellum-Animal Model Evidence. Cerebellum, 15(3), 285-298; LeDoux, M. (2005). Harmaline tremor. In Movement Disorders. Elsevier Inc.; Louis, E. D. (2014). Re-thinking the biology of essential tremor: From models to morphology. Parkinsonism and Related Disorders, 20 (SUPPL.1), S88-S93). This is suggested to result from direct modulation of the olivocerebellar circuit via enhanced coupling between inferior olivary neuros (Handforth 2016; Louis 2014). Clinical agents that suppress ET in patients are generally effective in the harmaline-induced tremor model (Handforth 2016). Following acute dosing of harmaline in mice, the tremor frequency (10-20 Hz) is typically recorded using an automated force plate-based system.

Procedure: Tremor activity was measured using a custom-made testing apparatus containing a piezoelectric sensing platform which was connected to an amplifier (A-M Systems model 1700). Adult male CD1 mice were brought to the experiment room for acclimatization 1 h prior to testing. Mice were first placed inside the testing apparatus for 10 min baseline habituation. After the initial habituation, mice were treated with vehicle (10 ml/kg) or test article (30 mg/kg, i.p.) and placed back in the apparatus for an additional 20 min baseline recording. Subsequently, animals were injected with harmaline (30 mg/kg, i.p.) and tremor activity was measured for 20 min. The electric signal of tremor activity was amplified at a sampling rate of 512 Hz with a multichannel amplifier and then acquired, quantized, and digitized by a data acquisition unit (CED Micro 1401). The software Spike2 (v 7.07, Cambridge Electronic Design, Cambridge, UK) was used to analyze the tremor signal by performing Fourier transformation of the data into frequency spectra. Changes in absolute power were expressed relative to each animal's 20-min baseline for the frequencies of 10-20 Hz. Results for selected compounds tested at 30 mg/kg (i.p.) are shown in the table below. Comparisons between treatment and respective vehicle groups were assessed with two-tailed, Student's t-test. The level of statistical significance was defined as p<0.05 (see, e.g., Table 2.1) The magnitude of tremor suppression in percent compared to vehicle is shown in Table 2.2.

TABLE 2.1
Results for Selected Compounds with Comparisons
Between Treatment and Respective Vehicle Groups
Assessed with Two-Tailed, Student's t-test

| | | Tremor power relative | P value |
|---|---|---|---|
| | | to baseline | (compound |
| Study | Compound | (mean ± s.e.m.) | vs vehicle) |
| 1 | Vehicle/Harmaline | 1050 ± 203 | — |
| | Compound 46/Harmaline | 145 ± 27 | p < 0.0005 |
| | Compound 45/Harmaline | 144 ± 35 | p < 0.0005 |
| | Compound 44/Harmaline | 518 ± 109 | p = 0.03 |
| 2 | Vehicle/Harmaline | 517 ± 56 | — |
| | Compound 11/Harmaline | 134 ± 32 | p < 0.0001 |
| | Compound 12/Harmaline | 196 ± 59 | p = 0.0007 |

A tremor suppression score was defined as the magnitude of tremor suppression in percent compared to vehicle: ≤24% (D), 25-55% (C), 56-70% (B) and 71-100% (A). Not tested (---).

TABLE 2.2
Harmaline Model Tremor Suppression Activity

| | Tremor |
|---|---|
| | Suppression |
| Compound | Activity |
| 1 | B |
| 2 | A |
| 3 | C |
| 4 | B |
| 5 | C |
| 6 | A |
| 7 | C |
| 8 | C |
| 9 | C |
| 10 | D |
| 11 | A |
| 12 | B |
| 13 | — |
| 14 | — |
| 15 | C |
| 16 | B |
| 17 | B |
| 18 | B |
| 19 | D |
| 20 | A |
| 21 | — |
| 22 | — |
| 23 | — |
| 24 | — |
| 25 | B |
| 26 | A |
| 27 | D |
| 28 | C |
| 29 | B |
| 30 | A |
| 31 | A |
| 32 | B |
| 33 | C |
| 34 | B |
| 35 | — |
| 36 | — |
| 37 | B |
| 38 | A |
| 39 | C |
| 40 | C |
| 41 | B |
| 42 | D |
| 43 | D |
| 44 | C |
| 45 | A |
| 46 | A |
| 47 | — |
| 48 | — |
| 49 | — |
| 50 | — |
| 51 | — |
| 52 | — |
| 53 | C |
| 54 | — |
| 55 | — |
| 56 | — |
| 57 | D |
| 58 | — |
| 59 | D |
| 60 | C |
| 65 | — |
| 66 | — |
| 67 | — |
| 68 | — |
| 69 | D |
| 70 | D |
| 71 | B |
| 72 | C |
| 73 | B |
| 74 | B |
| 75 | — |
| 76 | B |

Tacrine-induced Tremor: Cholinomimetic-induced tremulous jaw movements (TJMs) are considered a pharmacological model of parkinsonian resting tremor. TJMs are defined as rapid vertical deflections of the lower jaw that are not directed at any stimulus (Salamone et al., 1998). In rodents, TJMs can be induced by conditions that also result in parkinsonism in humans (i.e., striatal dopamine depletion, dopamine receptor antagonism and cholinomimetic activity) and reversed by several antiparkinsonian drugs including Levodopa, dopamine receptor agonists and anticholinergics (Collins-Praino, L. E., Paul, N. E., Rychalsky, K. L., Hinman, J. R., Chrobak, J. J., Senatus, P. B., & Salamone, J. D. (2011). Pharmacological and physiological characterization of the tremulous jaw movement model parkinsonian tremor: Potential insights into the pathophysiology of tremor. Frontiers in Systems Neuroscience, 5 (JULY 2011), 1-14). The cholinesterase inhibitor tacrine is frequently used to induce TJM in rats. Tacrine-induced TJMs occur in the same 3-7 Hz frequency range observed in parkinsonian resting tremor, which is distinct from that of dyskinesia (1-2 Hz), and postural tremor (8-14 Hz; Collins-Praino et al., 2011).

Procedure: Adult male Wistar rats were acclimated to the experimental room for at least 1 h. Animals were treated with vehicle (5 ml/kg) or test article (10-100 mg/kg, p.o.) and returned to their home cage for the designated pretreatment-time. Rats were then injected with Tacrine (5 mg/kg, i.p.) and placed in the testing chamber for an initial 10 min habituation period. Following habituation, TJMs were scored for 5 min by an experienced observer blinded to treatment. Multiple group comparisons were assessed with one-way ANOVA, followed by appropriate post-hoc analysis. The level of statistical significance was defined as p<0.05. Exemplary compounds disclosed herein were tested and showed tremor suppressing activity.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A compound of Formula I 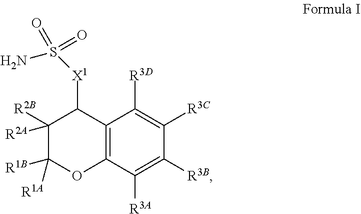
or a pharmaceutically acceptable salt thereof,
wherein:
X¹ is C(R^{4A})(R^{4B}) or N(R^{4C});
R^{4A} and R^{4B} are each independently H, (C₁-C₄)alkyl, (C₂-C₄)alkenyl, (C₂-C₄)alkynyl, (C₁-C₄)alkoxy, or (C₃-C₆)cycloalkyl; or
R^{4A} and R^{4B}, together with the carbon atom to which they are attached, form a (C₃-C₆)cycloalkyl;
R^{4C} is H, (C₁-C₄)alkyl, (C₂-C₄)alkenyl, (C₂-C₄)alkynyl, or (C₃-C₆)cycloalkyl;
R^{1A}, R^{1B}, R^{2A}, and R^{2B} are each independently H, halogen, (C₁-C₄)alkyl, (C₂-C₄)alkenyl, (C₂-C₄)alkynyl, (C₁-C₄)alkoxy, (C₁-C₄)haloalkyl, or (C₃-C₆)cycloalkyl;
or R^{1A} and R^{1B}, together with the carbon atom to which they are attached, form a spiro-(C₃-C₆)cycloalkyl;
or R^{2A} and R^{2B}, together with the carbon atom to which they are attached, form a spiro-(C₃-C₆)cycloalkyl; and
R^{3A}, R^{3B}, R^{3C}, and R^{3D} are each independently H, halogen, hydroxyl, (C₁-C₄)alkyl, (C₂-C₄)alkenyl, (C₂-C₄)alkynyl, (C₁-C₄)alkoxy, (C₁-C₄)haloalkyl, acyl, —CN, or (C₃-C₆)cycloalkyl;
with the provisos that:
when R^{1A}, R^{1B}, R^{2A}, R^{2B}, R^{3A}, R^{3B}, R^{3C}, and R^{3D} are each hydrogen, then (i) at least one of R^{4A} and R^{4B} is a group other than hydrogen or (ii) R^{4C} is a group other than hydrogen; and
when X¹ is N(R^{4C}) and only one of R^{1A}, R^{1B}, R^{2A}, R^{2B}, R^{3A}, R^{3B}, R^{3C}, R^{3D}, and R^{4C} is a group other than hydrogen, then (i) R^{1A} is not methyl; (ii) R^{1B} is not methyl; and (iii) R^{3C} is not methoxy or ethoxy.

2. The compound of claim 1, represented by Formula I(A) 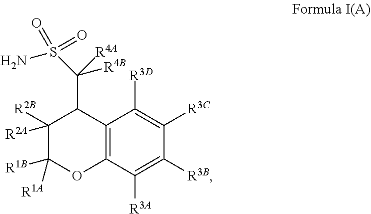
or a pharmaceutically acceptable salt thereof.

3. The compound of claim 1, represented by Formula I(B) 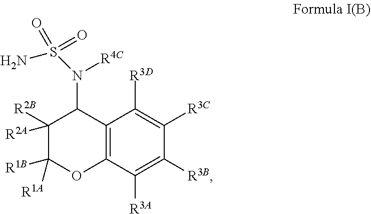
or a pharmaceutically acceptable salt thereof.

4. The compound of claim 1, or a pharmaceutically acceptable salt thereof, wherein:
a. R^{4A} and R^{4B} are each independently H or (C₁-C₄)alkyl; or
b. R^{4A} and R^{4B} are each independently H, methyl, or ethyl; or
c. R^{4A} is H, and R^{4B} is (C₁-C₄)alkyl; or
d. R^{4A} and R^{4B}, together with the carbon atom to which they are attached, form a (C₃-C₆)cycloalkyl; or
e. R^{4A} and R^{4B}, together with the carbon atom to which they are attached, form a cyclopropyl group.

5. The compound of claim 1, or a pharmaceutically acceptable salt thereof, wherein R^{4C} is H or (C₁-C₄)alkyl, optionally wherein R^{4C} is H or methyl.

6. The compound of claim 1, or a pharmaceutically acceptable salt thereof, wherein:
a. R^{1A} and R^{1B} are each independently H or (C₁-C₄)alkyl; or
b. R^{1A} and R^{1B} are each independently H, methyl, or ethyl; or
c. R^{1A} is H, and R^{1B} is (C₁-C₄)alkyl; or
d. R^{1A} and R^{1B}, together with the carbon atom to which they are attached, form a spiro-(C₃-C₆)cycloalkyl; or
e. R^{1A} and R^{1B}, together with the carbon atom to which they are attached, form a spiro-cyclopropyl group or a spiro-cyclobutyl group.

7. The compound of claim 1, or a pharmaceutically acceptable salt thereof, wherein:
a. R^{2A} and R^{2B} are each independently H or (C₁-C₄)alkyl; or
b. R^{2A} and R^{2B} are each independently H, methyl, or ethyl; or
c. R^{2A} is H, and R^{2B} is (C₁-C₄)alkyl.

8. The compound of claim 1, or a pharmaceutically acceptable salt thereof, wherein R^{3A} is H or halogen, optionally wherein R^{3A} is H or F.

9. The compound of claim 1, or a pharmaceutically acceptable salt thereof, wherein:
a. R^{3B} is H, halogen, (C₁-C₄)alkyl, (C₁-C₄)haloalkyl, or —CN; or
b. R^{3B} is halogen, (C₁-C₄)alkyl, (C₁-C₄)haloalkyl, or —CN; or
c. R^{3B} is H, F, Cl, methyl, CF₃, or —CN; or
d. R^{3B} is F.

10. The compound of claim 1, or a pharmaceutically acceptable salt thereof, wherein R^{3C} is H or halogen, optionally wherein R^{3C} is H or F.

11. The compound of claim 1, or a pharmaceutically acceptable salt thereof, wherein R^{3D} is H or halogen, optionally wherein R^{3D} is H or F.

12. The compound of claim 1, or a pharmaceutically acceptable salt thereof, wherein:
X¹ is C(R^{4A})(R^{4B}) or N(R^{4C});
R^{4A}, R^{4B}, and R^{4C} are each independently H or (C₁-C₄)alkyl.
or R^{4A} and R^{4B}, together with the carbon atom to which they are attached, form a (C₃-C₆)cycloalkyl;
R^{1A}, R^{1B}, R^{2A}, and R^{2B} are each independently H or (C₁-C₄)alkyl.
or R^{1A} and R^{1B}, together with the carbon atom to which they are attached, form a spiro-(C₃-C₆)cycloalkyl; and
R^{3A}, R^{3B}, R^{3C}, and R^{3D} are each independently H, halogen, (C₁-C₄)alkyl, (C₁-C₄)haloalkyl, or —CN;
with the provisos that:
when R^{1A}, R^{1B}, R^{2A}, R^{2B}, R^{3A}, R^{3B}, R^{3C}, and R^{3D} are each hydrogen, then (i) at least one of R^{4A} and R^{4B} is a group other than hydrogen or (ii) R^{4C} is a group other than hydrogen; and
when X¹ is N(R^{4C}) and only one of R^{1A}, RIB, R^{2A}, R^{2B}, R^{3A}, R^{3B}, R^{3C}, R^{3D}, and R^{4C} is a group other than hydrogen, then (i) R^{1A} is not methyl; and (ii) R^{1B} is not methyl.

13. The compound of claim 1, or a pharmaceutically acceptable salt thereof, wherein the compound is selected from a compound of Table 1.

14. The compound of claim 1, or a pharmaceutically acceptable salt thereof, wherein the compound is selected from: 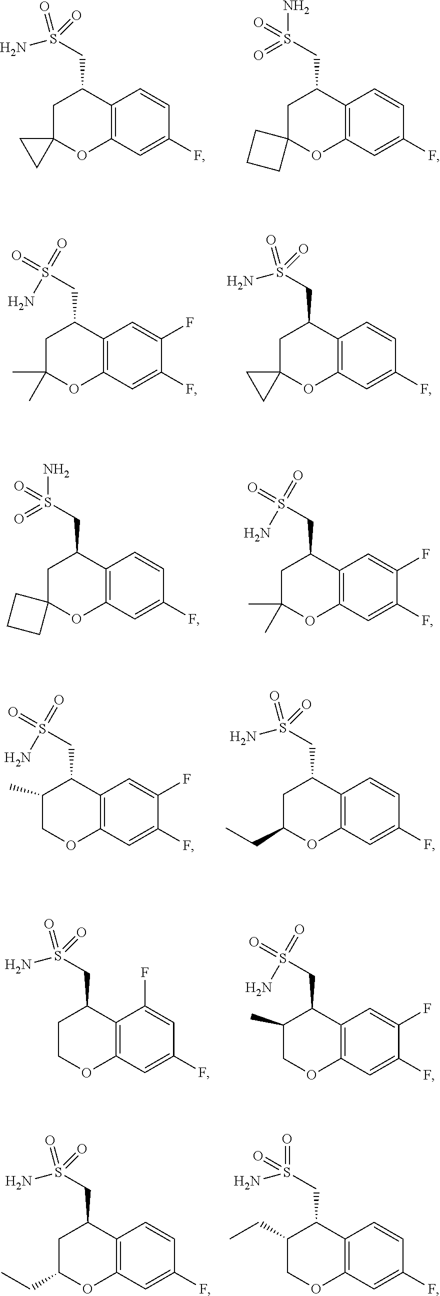 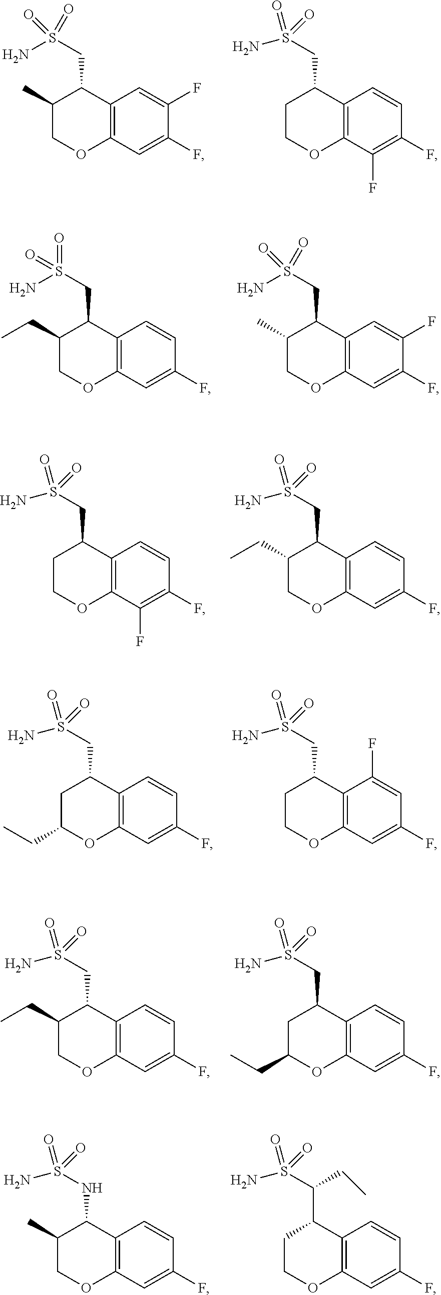 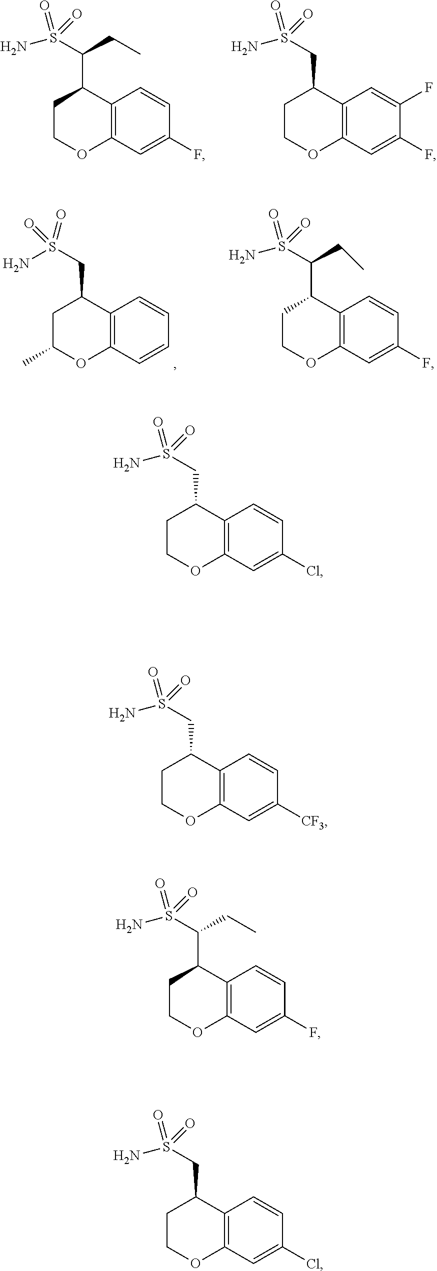 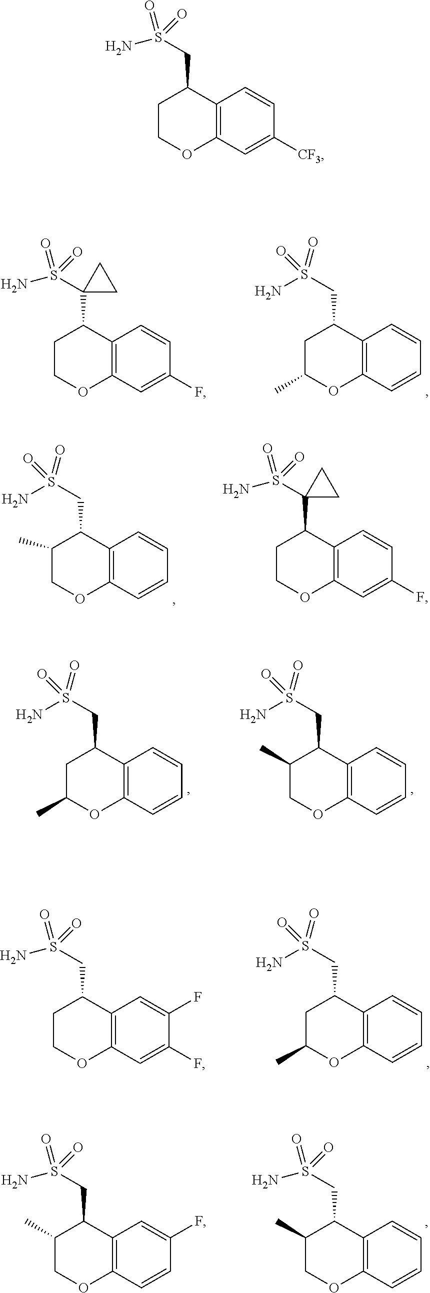 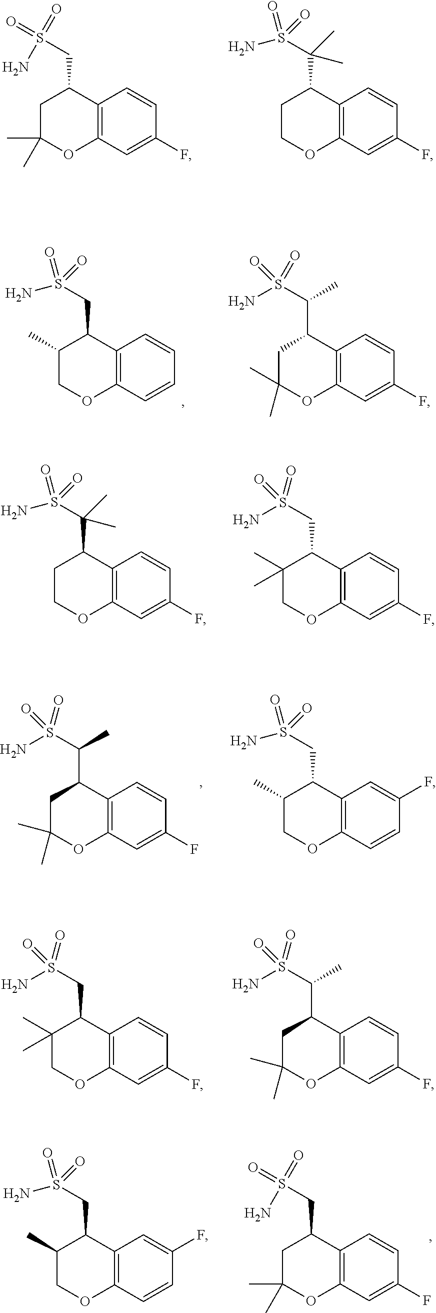 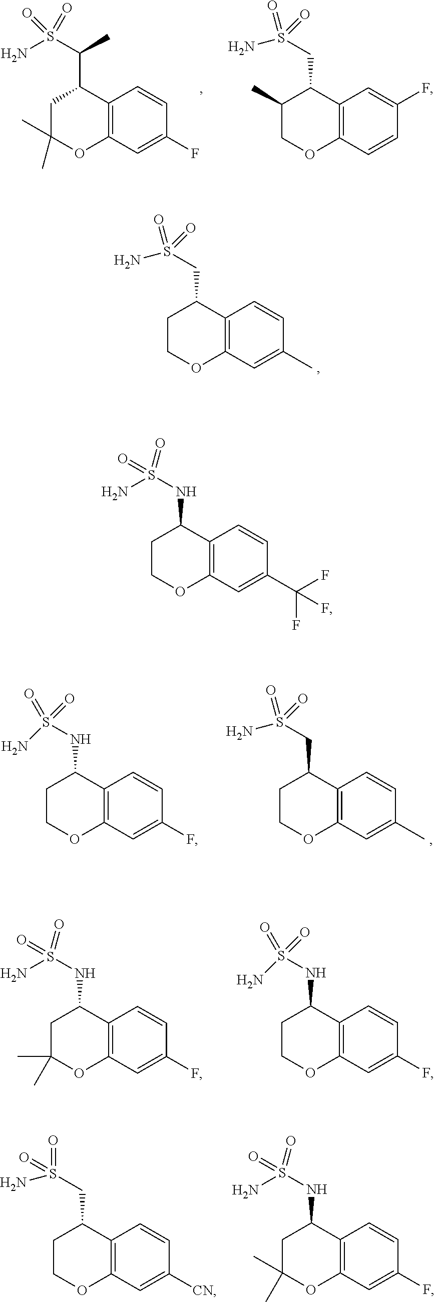 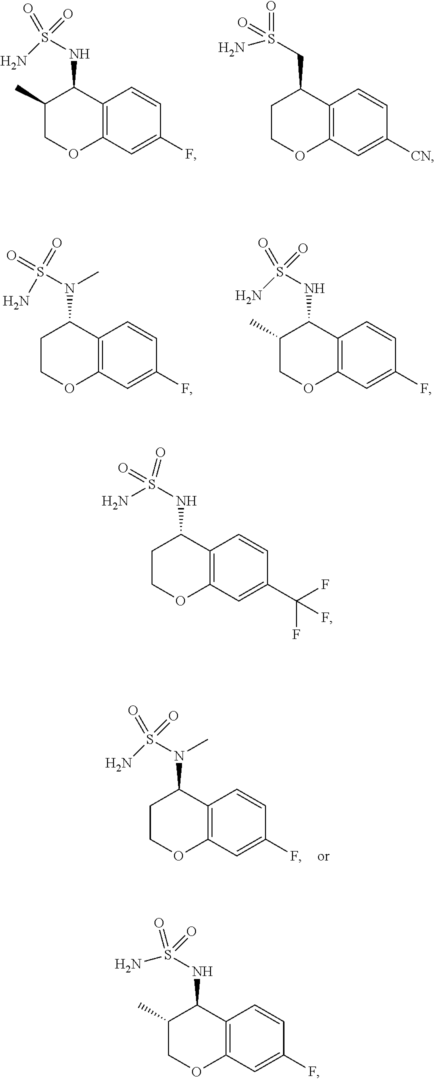
or a pharmaceutically acceptable salt thereof.

15. The compound of claim 14, or a pharmaceutically acceptable salt thereof, wherein the compound is selected from: 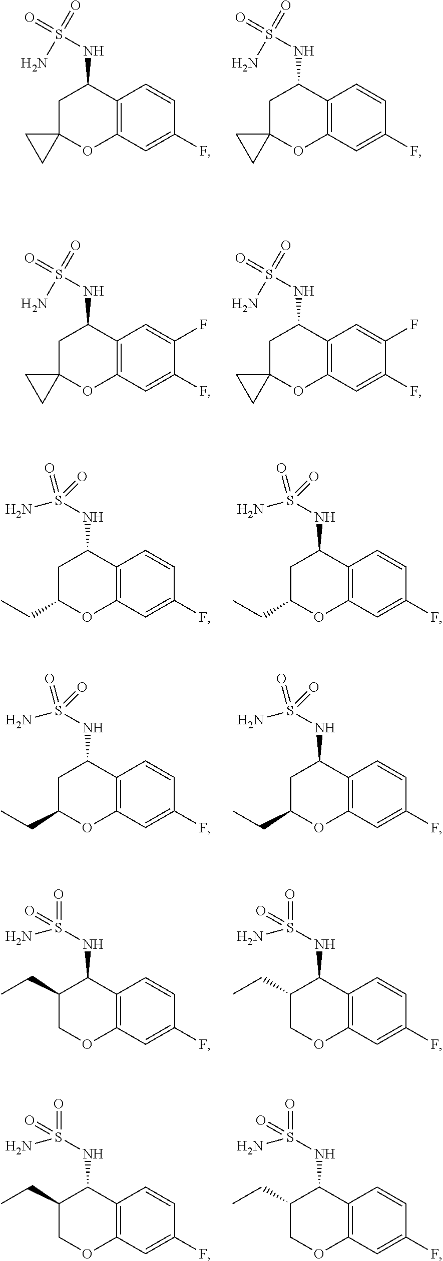 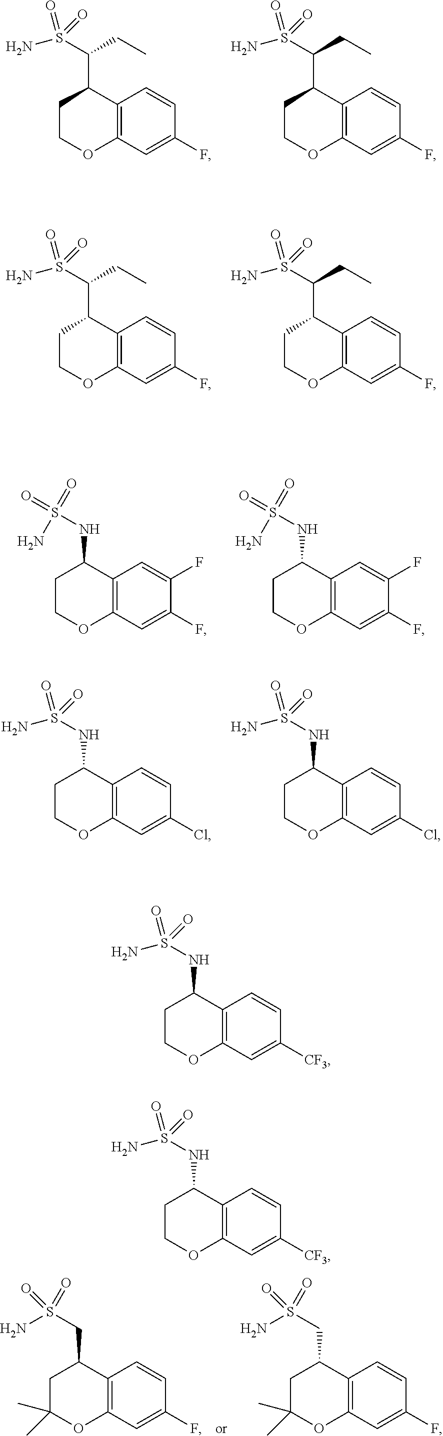
or a pharmaceutically acceptable salt thereof.

16. A pharmaceutical composition, comprising a therapeutically effective amount of a compound of claim 1, or a pharmaceutically acceptable salt thereof, and one or more pharmaceutically acceptable excipient.

17. A pharmaceutical combination, comprising a therapeutically effective amount of a compound of claim 1, or a pharmaceutically acceptable salt thereof, and a therapeutically effective amount of one or more other therapeutic agents.

18. A method of treating a neurological or psychiatric disease or disorder in a subject in need thereof, comprising administering to the subject a therapeutically effective amount of a compound according to claim 1, or a pharmaceutically acceptable salt thereof.

19. The method of claim 18, wherein the neurological or psychiatric disease or disorder is a movement disorder.

20. The method of claim 18, wherein the movement disorder is Tremor; Dyskinesia; Dystonia; Tics; Dysphonia; Ataxia (e.g., spinocerebellar ataxia); Myoclonus; Essential Tremor; Epilepsy; Tardive Dyskinesia; Restless Leg Syndrome; Tourette Syndrome; Multiple System Atrophy (MSA); Multiple Sclerosis; Huntington's Disease; Parkinson's Disease; Parkinsonism; Parkinson's disease tremor, Atypical Parkinsonisms; Wilson's Disease; or damage or side effect from Stroke.

21-22. (canceled)
